(19) United States
(12) Patent Application Publication
(10) Pub. No.: US 2017/0101624 A1
(43) Pub. Date: Apr. 13, 2017

(54) METHODS OF ENHANCING T-CELL LONGEVITY AND USES THEREOF

(71) Applicant: Washington University, St. Louis, MO (US)

(72) Inventors: Erika L. Pearce, St. Louis, MO (US); Michael D. Buck, St. Louis, MO (US); David O'Sullivan, St. Louis, MO (US)

(21) Appl. No.: 15/287,294

(22) Filed: Oct. 6, 2016

Related U.S. Application Data
(60) Provisional application No. 62/238,441, filed on Oct. 7, 2015.
(60) Provisional application No. 62/325,769, filed on Apr. 21, 2016.

(51) Int. Cl. C12N 5/0783; A61K 39/00

(52) U.S. Cl. CPC C12N 5/0636; A61K 39/0011; C12N 2501/999; A61K 2039/5158; C12N 2501/2302

(57) ABSTRACT

The present disclosure encompasses methods of enhancing T cell longevity and/or T cell function by promoting mitochondrial fusion and/or mitochondrial structural remodeling including cristae. Compositions comprising the enhanced T cells may be used in adoptive cellular immunotherapy.

### CROSS REFERENCE TO RELATED APPLICATIONS

This application claims the benefit of U.S. Provisional Application No. 62/238,441, filed Oct. 7, 2015 and U.S. Provisional Application No. 62/325,769, filed Apr. 21, 2016, each of the disclosures of which is hereby incorporated by reference in its entirety.

### GOVERNMENTAL RIGHTS

This invention was made with government support under R01 CA181125, R01 AI091965 and DGE-1143954 awarded by NIH and NSF. The government has certain rights in the invention.

### FIELD OF THE INVENTION

The present disclosure encompasses methods of enhancing T cell longevity and/or T cell function by promoting mitochondrial fusion and/or mitochondrial structural remodeling including cristae. Compositions comprising the enhanced T cells may be used in adoptive cellular immunotherapy.

### BACKGROUND OF THE INVENTION

Adoptive cellular immunotherapy uses a person's own isolated tumor-specific T cells or chimeric antigen receptor (CAR) T cells that are expanded in vitro and then transferred back into a patient to fight against a tumor. However, these cells often do not have long-term survival and thus ultimately fail to control a tumor long-term. Thus, there is a need in the art to create long-lived immune cells that will protect the body against infections and cancer.

### SUMMARY OF THE INVENTION

In an aspect, the disclosure provides a method to promote T-cell longevity. The method comprises culturing T cells in the presence of a composition comprising one or more compounds to promote mitochondrial fusion and/or mitochondrial structural remodeling.

In another aspect, the disclosure provides a method to improve adoptive cellular immunotherapy in a subject. The method comprises administering to the subject a therapeutic composition comprising T cells that have been cultured in the presence of a composition comprising one or more compounds to promote mitochondrial fusion.

In still another aspect, the disclosure provides a method to reduce tumor growth in a subject. The method comprises administering to the subject a therapeutic composition comprising T cells that have been cultured in the presence of a composition comprising one or more compounds to promote mitochondrial fusion.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C, FIG. 1D and FIG. 1E depict images and immunoblots showing that effector and memory T cells possess distinct mitochondrial morphologies. (FIG. 1A) C57BL/6 mice were infected i.p. with 1×10⁷ CFU LmOVA. Effector (T_{E}, CD44^{hi} CD62L^{lo}, 7 days post infection) and memory T (T_{M}, CD44^{hi}CD62L^{hi}, 21 days post infection) cells were sorted and analyzed by EM as well as (FIG. 1B) IL-2 T_{E} and IL-15 T_{M} cells generated from differential culture of OT-I cells activated with OVA peptide and IL-2 using IL-2 or IL-15, scale bar=0.5 μm. (FIG. 1C, FIG. 1D) Mitochondrial morphology was analyzed in live OT-I PhAM cells over time before and after αCD3/CD28 activation and differential cytokine culture by spinning disk confocal microscopy. Mitochondria are green (GFP) and nuclei are blue (Hoechst). (FIG. 1C) Scale bar=5 μm, (FIG. 1D) Scale bar=1 μm. (FIG. 1E) Immunoblot analysis of cell protein extracts from (FIG. 1C), probed for Mfn2, Opa1, Drp1, phosphorylated Drp1 at Ser616 (Drp1p^{S616}), and β-actin. Results representative of 2 experiments. See also FIG. 7.

FIG. 2A, FIG. 2B, FIG. 2C, FIG. 2D, FIG. 2E and FIG. 2F depict graphs, images and flow cytometry plots showing that memory T cell development and survival, unlike effectors, requires mitochondrial fusion. (FIG. 2A) Relative in vitro survival ratios of Mfn1, Mfn2, or Opa1 deficient (CD4 Cre⁺, −/−) to wild-type control (CD4 Cre⁻, +/+) OT-I IL-2 T_{E} and IL-15 T_{M} cells (*p=0.0465). Data normalized from 2-3 independent experiments shown as mean±SEM. (FIG. 2B) Mitochondrial morphology of OT-I Opa1 wild-type and Opa1 knockout IL-2 T_{E} and IL-15 T_{M} cells analyzed by EM (scale bar=0.5 μm, one experiment represented) and (FIG. 2C) Seahorse EFA. (Left) bar graph represents ratios of O₂ consumption rates (OCR, an indicator of OXPHOS) to extracellular acidification rates (ECAR, an indicator of aerobic glycolysis) at baseline and (right) spare respiratory capacity (SRC) (% max OCR after FCCP injection of baseline OCR) of indicated cells (*p<0.03, **p=0.0079). Data from 3 experiments shown as mean±SEM. (FIG. 2D, FIG. 2E, FIG. 2F) 10⁴ OT-I Opa1^{+/+} or Opa1^{-/-} T cells were transferred i.v. into C57BL/6 CD90.1 mice infected i.v. with 1×10⁷ CFU LmOVA. Blood was analyzed by flow cytometry at indicated time points post infection. After 21 days, mice were challenged i.v. with 5×10⁷ CFU LmOVA and blood analyzed post challenge (p.c.). (FIG. 2D) % Donor K^{b}/OVA⁺ and CD90.2⁺ live cells shown in representative flow plots and (FIG. 2E) line graph with mean±SEM. (*p=0.0238, **p<0.005). (FIG. 2F) Number of donor K^{b}/OVA⁺ cells isolated from spleens of infected mice shown as mean±SEM (*p=0.0126). (FIG. 2D, FIG. 2E, FIG. 2F) Results representative of 2 experiments (n=9-11 per group). See also FIG. 8.

FIG. 3A, FIG. 3B, FIG. 3C, FIG. 3D, FIG. 3E, FIG. 3F, FIG. 3G, FIG. 3H, FIG. 3I, FIG. 3J, FIG. 3K and FIG. 3L depicts a schematic, graphs and images showing that enhancing mitochondrial fusion promotes the generation of memory-like T cells. (FIG. 3A, FIG. 3B, FIG. 3C, FIG. 3D, FIG. 3E, FIG. 3F, FIG. 3I, FIG. 3J, FIG. 3K, FIG. 3L) OVA peptide and IL-2 activated OT-I cells were differentiated into IL-2 T_{E} or IL-15 T_{M} cells for 3 days in the presence of DMSO control or fusion promoter M1 and fission inhibitor Mdivi-1 (M1+Mdivi-1) as shown in (FIG. 3A) pictorially. (FIG. 3B) Representative spinning disk confocal images from 3 experiments of live cells generated from OT-I PhAM mice. Mitochondria are green (GFP) and nuclei are blue (Hoechst), scale bar=5 μm. (FIG. 3C) Cells stained with MitoTracker Green and analyzed by flow cytometry. Relative MFI (left) from 6 experiments shown as mean±SEM (*p=0.0394, **p=0.0019) with representative histograms (right). (FIG. 3D) Baseline OCR and SRC of indicated cells from 3-4 experiments shown as mean±SEM (*p=0.0485, ***p<0.0001). (FIG. 3E) CD62L expression analyzed by flow cytometry of indicated cells. Relative MFI (left) from 7 experiments shown as mean±SEM (*p=0.0325, **p=0.0019, ***p<0.0001) with representative histograms (right). (FIG. 3F) OCR of indicated cells at baseline and in response to PMA and ionomycin stimulation (PMA+iono), oligomycin (Oligo), FCCP, and rotenone plus antimycin A (R+A). Data represents 2 experiments shown as mean±SEM. (FIG. 3G, FIG. 3H) OT-I cells were transduced with either empty (Control), Mfn1, Mfn2, or Opa1 expression vectors, sorted, and cultured to generate IL-2 T_{E} cells. (FIG. 3G) Histograms representative of 4 experiments of cells stained for MitoTracker Deep Red and (FIG. 3H) OCR data at baseline of transduced cells from 2 experiments. (FIG. 3I, FIG. 3J, FIG. 3K, FIG. 3L) 1-2×10⁶ IL-2 T_{E} cells cultured with DMSO (gray diamonds) or M1+Mdivi-1 (blue squares) were transferred into congenic C57BL/6 recipient mice. Cell counts of donor cells recovered 2 days later from the (FIG. 3I) spleen (***p=0.005) and (FIG. 3J) peripheral lymph nodes (pLNs, ***p=0.0006). (FIG. 3K) Blood from recipient mice analyzed for % donor K^{b}/OVA⁺ cells post transfer and challenge with 1×10⁷ CFU LmOVA by flow cytometry (*p=0.0150, n=5 per group). (FIG. 3L) Donor K^{b}/OVA⁺ cells recovered from recipient spleens 6 days post challenge (*p=0.0383). (FIG. 3I, FIG. 3J, FIG. 3K, FIG. 3L) Data represents 2 experiments shown as mean±SEM. See also FIG. 9.

FIG. 4A, FIG. 4B, FIG. 4C, FIG. 4D and FIG. 4E depicts graphs and images showing that mitochondrial fusion improves adoptive cellular immunotherapy against tumors. (FIG. 4A, FIG. 4B) C57BL/6 mice were inoculated s.c. with 1×10⁶ EL4-OVA cells. (FIG. 4A) After 5 or (FIG. 4B) 12 days, 1×10⁶ or 5×10⁶ OT-I IL-2 T_{E} cells cultured with DMSO or M1+Mdivi-1 were transferred i.v. into recipient mice and tumor growth assessed. Data represents 2 experiments shown as mean±SEM (n=5 per group, *p<0.05, **p<0.005). (FIG. 4C, FIG. 4D, FIG. 4E) Human CD8⁺ PBMCs were activated with αCD3/CD28+IL-2 to generate IL-2 T_{E} cells. (FIG. 4C) Confocal images of indicated treated cells where mitochondria are green (MitoTracker) and nuclei are blue (Hoechst). Representative images from 2 of 4 biological donors, scale bar=5 μm. (FIG. 4D) OCR/ECAR ratios and SRC of indicated cells from 2 separate donors shown as mean±SEM (*p=0.0303, **p<0.005, ***p<0.0001). (FIG. 4E) MitoTracker Green staining and CD62L, CD45RO, and CCR7 expression analyzed by flow cytometry shown with representative histograms from 4-6 biological replicates. See also FIG. 10.

FIG. 5A, FIG. 5B, FIG. 5C, FIG. 5D and FIG. 5E depicts graphs showing that fusion promotes memory T cell metabolism, but Opa1 is not required for FAO. OCR measured at baseline and in response to media, etomoxir (Eto) and other drugs as indicated of (FIG. 5A) IL-2 T_{E} cells cultured in DMSO or M1+Mdivi-1, (FIG. 5B) control or Opa1 transduced IL-2 T_{E} cells, (FIG. 5C) Opa1^{+/+} and Opa1^{-/-} IL-2 T_{E} cells cultured in DMSO or M1+Mdivi-1 (FIG. 5D) or without drugs, and (FIG. 5E) ex vivo donor OT-I Opa1^{+/+} and Opa1^{-/-} day 7 T_{E} cells derived from LmOVA infection. Data representative of 2 independent experiments shown as mean±SEM (***p<0.0001). See also FIG. 11.

FIG. 6A, FIG. 6B, FIG. 6C, FIG. 6D, FIG. 6E and FIG. 6F depict graphs and images showing that mitochondrial cristae remodeling signals metabolic pathway engagement. (FIG. 6A) EM analysis of mitochondrial cristae from T_{E} and T_{M} cells isolated after LmOVA infection and (FIG. 6B) in vitro cultured IL-2 T_{E} and T_{M} cells. Data representative of 2 experiments, scale bar=0.25 μm. Relative proton leak (ΔOCR after oligomycin and subsequent injection of rotenone plus antimycin A) of (FIG. 6C) Opa1^{+/+} and Opa1^{-/-} IL-2 T_{E}, (FIG. 6D) infection elicited T_{E} and T_{M}, and (FIG. 6E) IL-2 T_{E} and IL-15 T_{M} cells. (FIG. 6C, FIG. 6D, FIG. 6E) Data combined from 2-4 experiments shown as mean±SEM (p**<0.005, ***p<0.0001). (FIG. 6F) Immunoblot analysis of ER protein Calnexin and ETC complexes (CI-NDUFB8, CII-SDHB, CIII-UQCRC2, CIV-MTC01, CV-ATP5A). Equivalent numbers of IL-2 T_{E} and IL-15 T_{M} cells were lysed in native lysis buffer followed by digitonin solubilization of intracellular membranes. Pellet (P) and solubilized supernatant (S) fractions were resolved on a denaturing gel. Data representative of 2 experiments. See also FIG. 12.

FIG. 7 depicts a schematic showing the in vitro differentiation of IL-2 T_{E} and IL-15 T_{M} cells approximate T cell response conditions that generate T_{E} and T_{M} cells in vivo (Related to FIG. 1). OT-I cells were activated with IL-2 and either OVA peptide or αCD3/CD28 for 3 days and then differentially cultured in IL-2 or IL-15 for an additional 3 days to generate IL-2 T_{E} and IL-15 T_{M} cells, respectively.

FIG. 8A, FIG. 8B, FIG. 8C, FIG. 8D and FIG. 8E depicts graphs, immunoblots and flow cytometry plots showing the assessment of genetic deletion of mitochondrial fusion proteins in IL-2 T_{E} and IL-15 T_{M} cells and of donor T_{E} cells generated from infection. (Related to FIG. 2). (FIG. 8A, FIG. 8B, FIG. 8C) IL-2 T_{E} and IL-15 T_{M} cells were cultured from (FIG. 8A) OT-I MfnI floxed, (FIG. 8B) OT-I Mfn2 floxed, (FIG. 8C) OT-I Opa1 floxed mice crossed to CD4 Cre transgenic mice to generate T cells conditionally deleted for proteins that mediate mitochondrial fusion (+/+ are CD4 Cre⁻ and −/− are CD4 Cre+). Efficiency of deletion by cre recombinase analyzed by (FIG. 8A) qPCR and (FIG. 8B, FIG. 8C) immunoblot. (FIG. 8D) Flow cytometry analysis of short-lived effector cells (SLEC, KLRG1^{hi} CD127^{lo}) and memory precursor effector cells (MPEC, KLRG1^{lo} CD127^{hi}) generated at day 7 post infection from OT-I Opa1^{+/+} and OT-I Opa1^{-/-} cells transferred into congenic recipients infected with LmOVA. Representative flow dot plots (left) and scatter dot plots (right) with mean±SEM bars. Each dot represents individual mice (n=8-9 per genotype), ***p<0.0001. (FIG. 8E) OCR analysis of day 10 post-infection OT-I Opa1^{+/+} and Opa1 donor cells at baseline and after oligomycin (Oligo), FCCP, and rotenone plus antimycin A (R+A) injections. Data is representative of 2 experiments shown as mean±SEM.

FIG. 9A, FIG. 9B, FIG. 9C, FIG. 9D, FIG. 9E, FIG. 9F, FIG. 9G and FIG. 9H depicts graphs showing the assessment of T cell phenotype and metabolism following pharmacological or enhancement of mitochondrial fusion (Related to FIG. 3). (FIG. 9A, FIG. 9B, FIG. 9C, FIG. 9D, FIG. 9E) IL-2 T_{E} and IL-15 T_{M} cells generated from OT-I mice were treated with DMSO control or M1+Mdivi-1. (FIG. 9A) ECAR of indicated cells at baseline and after PMA and ionomycin (PMA+iono) stimulation, oligomycin (Oligo), FCCP, and rotenone plus antimycin A (R+A). (FIG. 9B) qPCR analysis of relative mitochondrial DNA (mtDNA) to nuclear DNA (nDNA) ratios of indicated cells. (FIG. 9C) ECAR (left) and OCR/ECAR ratios (right) of indicated cells under basal conditions. (FIG. 9D) Histograms of membrane potential (CMxROS, TMRM) and mitochondrial ROS (MitoSOX) using indicated fluorescent dyes and (FIG. 9E) KLRG1, CD127, CCR7, and CD25 surface marker expression of indicated cells analyzed by flow cytometry. (FIG. 9F, FIG. 9G, FIG. 9H) OT-I IL-2 T_{E} cells were activated and transduced with empty vector (Control), MfnI, Mfn2, or Opa1 expressing retrovirus. (FIG. 9F) ECAR, OCR/ECAR, and SRC analyzed by Seahorse EFA, (FIG. 9G) KLRG1, CD127, CCR7, CD25 and PD-1 surface marker expression assessed by flow cytometry, and (FIG. 9H) gene expression analysis by qPCR. (FIG. 9A, FIG. 9B, FIG. 9C, FIG. 9D, FIG. 9E, FIG. 9F, FIG. 9G) Data are shown as mean±SEM and are representative or (FIG. 9B, FIG. 9C, FIG. 9F) combined from 2-3 experiments, not significant (ns), “p<0.001, ***p<0.0001.

FIG. 10A, FIG. 10B, FIG. 10C and FIG. 10D depict graphs and flow cytometry plots showing the examination of mouse and human IL-2 T_{E} cells after enforcing mitochondrial fusion with drugs (Related to FIG. 4). (FIG. 10A, FIG. 10B, FIG. 10C) Flow cytometry analyses of IL-2 T_{E} cells previously cultured with DMSO or M1+Mdivi-1 combined from 3 biological replicates. Cells were not subjected to further treatment with DMSO or M1+Mdivi-1 during experiment assays. (FIG. 10A) Cytolysis of EL4-OVA target cells at indicated concentrations. (FIG. 10B) Proliferation after restimulation with αCD3/CD28. (FIG. 10C) Intracellular cytokine staining after 4 hours stimulation with PMA and ionomycin. Relative MFI (left) with mean±SEM and representative contour plots (right) with percentage of cytokine positive cells indicated in gated cells and MFI in bold, *p<0.05. Gates based on unstimulated cells (not shown). (FIG. 10D) Human CD8+ PBMCs were activated with αCD3/CD28+IL-2 to generate IL-2 T_{E} cells and subjected to DMSO or M1+Mdivi-1 treatment. KLRG1, CD127, CD45RA, and CD25 surface marker expression analyzed by flow cytometry shown with representative histograms from 4-6 biological replicates.

FIG. 11A, FIG. 11B and FIG. 11C depict graphs showing bioenergetics analysis after promoting mitochondrial fusion in T cells and macrophages (Related to FIG. 5). (FIG. 11A) OCR of IL-15 T_{M} DMSO or M1+Mdivi-1 treated cells measured at baseline and in response to media, etomoxir (Eto), oligomycin (Oligo), FCCP, and rotenone plus antimycin A (R+A). (FIG. 11B) Bone marrow derived macrophages were cultured overnight with IL-4 (M2) or without (M0) and either DMSO or M1+Mdivi-1 overnight. OCR analyzed at baseline and after injection of mitochondrial inhibitors as indicated. (FIG. 11C) Baseline ECAR relative to DMSO controls of IL-2 T_{E} Opa1^{+/+} and Opa1^{-/-} cells cultured in DMSO or M1+Mdivi-1. Data are shown as mean±SEM and are representative of 2-3 experiments.

FIG. 12 depicts immunoblot analysis of ETC complexes (CI-NDUFB8, CII-SDHB, CIII-UQCRC2, CIV-MTC01, CV-ATP5A) and OMM protein Tom20. Equivalent numbers of IL-2 T_{E} and IL-15 T_{M} cells lysed in native lysis buffer followed by digitonin solubilization of intracellular membranes. Pellet (P) and solubilized supernatant (S) fractions were resolved on a denaturing gel. Second experiment represented from FIG. 6F.

### DETAILED DESCRIPTION OF THE INVENTION

A transition from aerobic glycolysis to mitochondrial fatty acid oxidation (FAO) is required for the development of CD8 memory T cells. The factors that drive this change in metabolism remain incompletely understood. Mitochondrial fusion and fission are dynamic processes that govern efficient cellular metabolism, as well as mitochondrial biogenesis, repair, and death. The inventors have found that pharmacological promotion of mitochondrial fusion in CD8 effector T cells induces properties characteristic of CD8 memory T cells, including increased spare respiratory capacity, mitochondrial mass, fatty acid oxidation (FAO), cristae morphology, electron transport chain (ETC) activity, cytokine production, and/or enhanced survival in vivo. Furthermore, promoting mitochondrial fusion provides a tractable way to improve adoptive cellular immunotherapy. The inventors have shown that adoptive transfer of these modified CD8 effector T cells reduced growth of acute and aggressive tumors in vivo. Various compositions and methods of the disclosure are described herein below.

### I. Compositions

In a first aspect, the present disclosure provides a composition comprising one or more compounds to promote mitochondrial fusion and/or mitochondrial structural remodeling.

In a second aspect, the present disclosure provides a therapeutic composition comprising isolated T cells that have been cultured in the presence of a composition comprising one or more compounds to promote mitochondrial fusion.

### (a) Composition Comprising One or More Compounds to Promote Mitochondrial Fusion

Mitochondrial fusion and fission are dynamic processes that govern efficient cellular metabolism, as well as mitochondrial biogenesis, repair and death. Mitochondrial fission and fusion processes are both mediated by large guanosine triphosphatases (GTPases) in the dynamin family that are well conserved between yeast, flies, and mammals. Their combined actions divide and fuse the two lipid bilayers that surround mitochondria. Fission is mediated by a cytosolic dynamin family member (Drp1 in worms, flies, and mammals and Dnm1 in yeast). Fusion between mitochondrial outer membranes is mediated by membrane-anchored dynamin family members named mitofusin 1 (Mfn1) and mitofusin 2 (Mfn2) in mammals, whereas fusion between mitochondrial inner membranes is mediated by a single dynamin family member called optic atrophy 1 (Opa1) in mammals or Mgm1 in yeast. In addition to promoting mitochondrial fusion, Opa1 has been shown to regulate apoptosis by controlling cristae remodeling and cytochrome c redistribution. Mitochondrial fission and fusion machineries are regulated by proteolysis and posttranslational modifications. Fusion rescues stress by allowing functional mitochondria to complement dysfunctional mitochondria by diffusion and sharing of components between organelles. Mitochondrial fusion can therefore maximize oxidative capacity in response to toxic stress. Fission and fusion events also regulate metabolism, longevity and cell fitness.

In an embodiment, a composition of the disclosure comprises one or more compounds that induces mitochondrial membrane remodeling. Additionally, a composition of the disclosure comprises one or more compounds that induces mitochondrial remodeling including cristae. For example, a compound can alter the localization or movement of mitochondria along cytoskeletal structures. Such an alteration can result in changes in mitochondrial dynamics. Further a compound can alter Kinesins, dynamin motor proteins, or receptor/adaptor proteins (such as Miro 1/2 and Trak1/2). Such proteins are associated with tethering motor proteins to the mitochondria surface and cause alterations in mitochondria localization or structure. In another embodiment, a compound can directly alters interaction of Kinesins, dynamin motor proteins or their receptor/adaptor proteins with fission and fusion machinery (e.g. MFNs) which can lead to alterations in mitochondria localization or structure. Still further, a compound that induces inner mitochondrial membrane remodeling can be a compound that affects calcium signaling. Additionally, MFF and/or Fis1 may be targeted to induce inner mitochondrial membrane remodeling.

In an embodiment, a composition of the disclosure comprises one or more compounds to promote mitochondrial fusion. In another embodiment, a composition of the disclosure comprises one or more compounds to promote mitochondrial fusion and/or to promote mitochondrial remodeling. In one embodiment, a compound that promotes mitochondrial fusion may be a compound that enhances the activity of one or more of Mfn1, Mfn2 and Opa1 (Mgm1). A compound that enhances the activity of Opa1 may also promote mitochondrial remodeling. In another embodiment, a compound that promotes mitochondrial fusion may be a compound that induces mitochondrial elongation. In still another embodiment, a compound that promotes mitochondrial fusion may be a compound that increases mitochondrial connectivity and integrity. In a different embodiment, a compound that promotes mitochondrial fusion may be a compound that increases ATP5A/B protein levels. In yet another embodiment, a compound that promotes mitochondrial fusion may be a compound that protects cells from MPP₊ induced mitochondrial fragmentation and cell death. In certain embodiments, a compound that promotes mitochondrial fusion may be a compound comprising a hydrazone or acylhydrazone moiety. For example, see compounds 1-17 as disclosed in Wang et al. Angew Chem Int Ed 2012; 51: 9302-9305, which is hereby incorporated by reference in its entirety. In a specific embodiment, a compound that promotes mitochondrial fusion may be hydrazone M1.

In other embodiments, a compound that promotes mitochondrial fusion may be a compound that inhibits mitochondrial fission (i.e. division). For example, compounds disclosed in WO 2012158624, the disclosure of which is hereby incorporated by reference in its entirety, may be used. In one embodiment, a compound that inhibits mitochondrial fission may be a compound that selectively inhibits the mitochondrial division dynamin. In another embodiment, a compound that inhibits mitochondrial fission may be a compound that inhibits Drp1 (Dnm1). Non-limiting examples of inhibitors of Drp1 include compounds disclosed in US 2005/0038051, US 2008/0287473 and Cassidy-Stone et al. Developmental Cell 2008; 14(2): 193-204, the disclosures of which are hereby incorporated by reference in their entireties. In still another embodiment, a compound that inhibits mitochondrial fission may be a compound that attenuates Drp1 (Dnm1) self-assembly. In yet another embodiment, a compound that inhibits mitochondrial fission may be a compound that causes the formation of mitochondrial net-like structures. In still another embodiment, a compound that inhibits mitochondrial fission may be a compound that retards apoptosis by inhibiting mitochondrial outer membrane permeabilization. In a different embodiment, a compound that inhibits mitochondrial fission may be a compound that blocks Biol-activated Bax/Bak-dependent cytochrome c release from mitochondria. In certain embodiments, a compound that inhibits mitochondrial fission may be a compound comprising a quinazoline moiety with an unblocked sulfhydryl moiety on the 2-position of the quinazolinone and limited rotation about the 3-position nitrogen-phenyl bond. For example, see compounds A, B, C, D, E, F, G, H, and I as disclosed in Cassidy-Stone et al. Developmental Cell 2008; 14(2): 193-204, which is hereby incorporated by reference in its entirety. Specifically, compound A (Mdivi-1) and compound B are said to have full efficacy relative to Mdivi-1, compounds C, D, and E are said to have moderate efficacy and compounds F, G, H and I are said to have poor efficacy. In a specific embodiment, a compound that inhibits mitochondrial fission may be Mdivi-1.

In certain embodiments, a composition of the disclosure comprises one or more compounds to promote mitochondrial fusion. For example, a composition of the disclosure may comprise 1, 2, 3, 4, 5, 6, 7, 8, 9 or 10 compounds to promote mitochondrial fusion. In a specific embodiment, a composition of the disclosure comprises two compounds to promote mitochondrial fusion. In one embodiment, a first compound may be a compound that promotes mitochondrial fusion and a second compound may be a compound that inhibits mitochondrial fission. In a specific embodiment, a composition of the disclosure comprises M1 and Mdivi-1.

(b) Therapeutic Composition Comprising Isolated T Cells that have been Cultured in the Presence of a Composition Comprising One or More Compounds to Promote Mitochondrial Fusion

In an embodiment, a composition of the disclosure comprises isolated T cells that have been cultured in the presence of a composition comprising one or more compounds to promote mitochondrial fusion. The composition comprising one or more compounds to promote mitochondrial fusion is described in Section I(a). As used herein, a “T cell”, which may be used interchangeably with “T lymphocyte”, is a type of lymphocyte that plays a central role in cell-mediated immunity. T cells can be distinguished from other lymphocytes, such as B cells and natural killer (NK) cells, by the presence of a T-cell receptor (TCR) on the cell surface. In general, T cells mature in the thymus. There are several types of T cells including: T helper cells (T_{H} cells), cytotoxic or effector T cells (T_{C} cells, T_{E} cells or CTLs), memory T cells (T_{M} cells), suppressor T cells (T_{reg} cells), natural killer T cells (NKT cells), mucosal associated invariant T cells, and gamma delta T cells (γδ T cells). T_{E} cells may be identified by CD44^{hi}CD62L^{lo}. T_{M} cells may be identified by CD44^{hi}CD62L^{hi}. In a specific embodiment, the T cells are T_{E} cells. Still further, the T cells may be CAR T cells.

T cells for use in a composition of the disclosure may be derived from a publically available cell line. For example, T cells may be obtained from STEMCELL™ T cell lines such as #70024 or T cells may be derived from the ATCC™ cell lines PCS-800-011 or PCS-800-013, which are primary mononuclear cell lines. Methods standard in the art may be used to isolate/enrich T cells from a cell line. For example, flow cytometry using cell surface markers may be used to isolate/enrich T cells. Optionally, prior to isolation, T cell growth and differentiation may be stimulated during cell culture with various factors. For example, IL2, IL15, IL7, anti-CD3 and/or anti-CD28 may be utilized to stimulate T cell growth. Alternatively, T cells for use in a composition of the disclosure may be isolated from a subject. The T cells may be obtained from a single subject, or a plurality of subjects. A plurality refers to at least two (e.g., more than one) subjects. When T cells obtained are from a plurality of subjects, their relationships may be autologous, syngeneic, allogeneic, or xenogeneic. In a specific embodiment, the relationship is allogeneic. In another specific embodiment, the relationship is autologous. Methods of collecting/isolating T cells from a subject are standard in the art. For example, several kits are commercially available to isolate T cells from whole blood or peripheral blood mononuclear cells (PBMCs). Additionally, flow cytometry using cell surface markers may be used to isolate/enrich T cells.

Isolation of T cells may result in a substantially pure population of T cells. The term “substantially pure”, may be used herein to describe a purified population of T cells that is enriched for T cells, but wherein the population of T cells are not necessarily in a pure form. Accordingly, a “substantially pure T cell population” refers to a population of T cells that is at least about 50%, preferably at least about 75-80%, more preferably at least about 85-90%, and most preferably at least about 95% of the cells making up the total cell population. Thus, a “substantially pure T cell population” refers to a population of T cells that contain fewer than about 50%, preferably fewer than about 20-25%, more preferably fewer than about 10-15%, and most preferably fewer than about 5% of cells that are not T cells.

Following isolation of T cells, T cells may be cultured in the presence of a composition comprising one or more compounds to promote mitochondrial fusion. Methods of culturing cell lines are standard in the art. For example, T cells may be cultured in the presence of a composition comprising one or more compounds to promote mitochondrial fusion for 1 or more days. Accordingly, T cells may be cultured in the presence of a composition comprising one or more compounds to promote mitochondrial fusion for 1, 2, 3, 4, 5, 6, 7, 8, 9, 10, 11, 12, 13 or 14 days. In certain embodiments, T cells may be cultured in the presence of a composition comprising one or more compounds to promote mitochondrial fusion for about 1 to about 5 days. In a specific embodiment, T cells may be cultured in the presence of a composition comprising one or more compounds to promote mitochondrial fusion for about 3 days.

In addition to the one or more compounds to promote mitochondrial fusion, the T cells may be cultured in the presence of a basal medium. The basal medium may contain a mixture of additional factors such as cytokines and growth factors. In certain embodiments, the additional factors may be selected from the group consisting of IL2, IL15 and IL7. In a specific embodiment, the additional factor is IL2. IL2 promotes the expansion of T cells. In another specific embodiment, the additional factor is IL15 or IL7. IL15 stimulates the proliferation of memory T cells. IL15 or IL7 allow substantial population expansion and improved T cell survival. In one embodiment, the basal medium includes amino acids, carbon sources (e.g., pyruvate, glucose, etc.), vitamins, serum proteins (e.g., albumin), inorganic salts, divalent cations, antibiotics, buffers, and other preferably defined components that support growth of T cells. Suitable basal mediums include, without limitation, RPMI medium, Iscove's medium, minimum essential medium, Dulbecco's Modified Eagles Medium, and others known in the art. The formulations of these and other mediums will be apparent to the skilled artisan.

In certain embodiments, the isolated T cells may be cultured in the absence of one or more compounds to promote mitochondrial fusion for one or more days prior to the addition of one or more compounds to promote mitochondrial fusion. For example, the isolated T cells may be cultured in the absence of one or more compounds to promote mitochondrial fusion for 1, 2, 3, 4, 5, 6, 7, 8, 9, 10, 11, 12, 13 or 14 days prior to the addition of one or more compounds to promote mitochondrial fusion. In certain embodiments, the isolated T cells may be cultured in the absence of one or more compounds to promote mitochondrial fusion for about 1 to about 5 days prior to the addition of one or more compounds to promote mitochondrial fusion. In a specific embodiment, the isolated T cells may be cultured in the absence of one or more compounds to promote mitochondrial fusion for about 3 days prior to the addition of one or more compounds to promote mitochondrial fusion.

Prior to the addition of the one or more compounds to promote mitochondrial fusion, the T cells may be cultured in the presence of a basal medium. The basal medium may contain a mixture of additional factors such as cytokines and growth factors. In certain embodiments, the additional factors may be selected from the group consisting of IL2, anti-CD3 and/or anti-CD28. In a specific embodiment, the additional factor is IL2. In another specific embodiment, the additional factors are anti-CD3 and anti-CD28. Additionally, the basal medium may contain antigen. The antigen may be included to generate antigen-specific T cells. For example, a tumor associated antigen or a viral antigen may be used to generate antigen-specific T cells. A skilled artisan would be able to select the antigen based on the desired disease or disorder to be treated. In an exemplary embodiment, the antigen is Ova peptide. In one embodiment, the basal medium includes amino acids, carbon sources (e.g., pyruvate, glucose, etc.), vitamins, serum proteins (e.g., albumin), inorganic salts, divalent cations, antibiotics, buffers, and other preferably defined components that support growth of T cells. Suitable basal mediums include, without limitation, RPMI medium, Iscove's medium, minimum essential medium, Dulbecco's Modified Eagles Medium, and others known in the art. The formulations of these and other mediums will be apparent to the skilled artisan.

The T cells may be used directly or may be frozen for use at a later date. A variety of mediums and protocols for freezing cells are known in the art. Generally, the freezing medium comprises 5-10% dimethyl sulfoxide (DMSO), 10-50% serum, and 50-90% culture medium.

i. Therapeutic Composition

Following culture in the presence of a composition comprising one or more compounds to promote mitochondrial fusion, the T cells may be combined with pharmaceutical carriers/excipients known in the art to enhance preservation and maintenance of the cells prior to administration. Accordingly, the T cells may be formulated into a therapeutic composition. As such, the disclosure encompasses a therapeutic composition comprising ex vivo T cells, wherein the T cells were cultured in the presence of a composition comprising one or more compounds to promote mitochondrial fusion.

In an aspect, a method of preparing a therapeutic composition for administration to a subject comprises culturing isolated T cells in the presence of a composition comprising one or more compounds to promote mitochondrial fusion and resuspending the T cells in a pharmaceutically acceptable medium suitable for administration to a recipient subject.

Pharmaceutically acceptable mediums suitable for administration to a subject are known in the art. In some embodiments, cell compositions of the disclosure can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene, glycol, liquid polyethylene glycol, and the like) and suitable mixtures thereof.

Sterile injectable solutions can be prepared by incorporating the T cells of the present disclosure in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, may be added. Prevention of the action of microorganisms can be ensured by various antibacterial and antifungal agents, for example, parabens, chlorobutanol, phenol, sorbic acid, and the like. The compositions can be isotonic, i.e., they can have the same osmotic pressure as blood and lacrimal fluid. The desired isotonicity of the compositions of this disclosure may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

In another aspect, the T cells are cryopreserved in a cryopreservation medium. The T cells may be cryopreserved prior to resuspending in a pharmaceutically acceptable medium. Alternatively, the starting cell population of T cells may be cryopreserved prior to culturing in the presence of a composition comprising one or more compounds to promote mitochondrial fusion. A variety of mediums and protocols for freezing cells are known in the art. Generally, the freezing medium comprises 5-10% dimethyl sulfoxide (DMSO), 10-50% serum, and 50-90% culture medium. Preferably, the freezing medium comprises 5-10% DMSO, 10-20% serum, and 70-85% culture medium. Other additives useful for preserving cells include, by way of example and not limitation, disaccharides such as trehalose (Scheinkonig, C. et al., Bone Marrow Transplant. 34(6):531-6 (2004)), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% DMSO, and 2% hetastarch. Other compositions and methods for cryopreservation are well known and described in the art (see, e.g., Broxmeyer, H. E. et al., Proc. Natl. Acad. Sci. USA 100(2). 645-650 (2003)). Cells are preserved at a final temperature of less than about −135° C.

### II. Methods

In an aspect, the disclosure provides a method to promote T-cell longevity. The method comprises culturing T cells in the presence of a composition comprising one or more compounds to promote mitochondrial fusion. Additionally, the aforementioned method promotes T cell function. Longevity and/or function may be measured by increased spare respiratory capacity, mitochondrial mass, fatty acid oxidation (FAO), cristae morphology, electron transport chain (ETC) activity, cytokine production, and/or enhanced survival in vivo. As demonstrated herein, culturing T cells in the presence of a composition comprising one or more compounds to promote mitochondrial fusion promotes T-cell longevity and function relative to T cells not cultured in the presence of a composition comprising one or more compounds to promote mitochondrial fusion. For example, T cells cultured in the presence of a composition comprising one or more compounds to promote mitochondrial fusion may persist in significantly greater numbers relative to T cells not cultured in the presence of a composition comprising one or more compounds to promote mitochondrial fusion for about 1, about 2, about 3, about 4, about 5, about 6 days, or about 7 days. A significant different may be measured using p-value. For instance, when using p-value, an increase in T cells cultured in the presence of a composition comprising one or more compounds to promote mitochondrial fusion relative to T cells not cultured in the presence of a composition comprising one or more compounds to promote mitochondrial fusion occurs when the p-value is less than 0.1, preferably less than 0.05, more preferably less than 0.01, even more preferably less than 0.005, the most preferably less than 0.001.

In another aspect, the disclosure provides a method to improve adoptive cellular immunotherapy in a subject. The method comprises administering to a subject a therapeutic composition comprising isolated T cells that have been cultured in the presence of a therapeutic composition comprising one or more compounds to promote mitochondrial fusion. As used herein, “adoptive cellular immunotherapy”, also referred to as “ACI”, is a T cell based immunotherapy whereby T cells are taken from a subject and stimulated and/or genetically manipulated. Following population expansion, the T cells are then transferred back into the subject. Accordingly, the methods of the disclosure may be used to treat a disease or disorder in which it is desirable to increase the number of T cells. For example, cancer and chronic viral infections. Regarding viral infections, ACI of virus-specific T cells of the disclosure may restore virus-specific immunity in a subject to prevent or treat viral diseases. Accordingly, virus-specific T cells of the disclosure may be used to reconstitute antiviral immunity after transplantation and/or to treat active viral infections. In a specific embodiment, a subject receiving T cells of the disclosure for treatment or prevention of a viral infection may be immunodeficient. Additionally, the methods of the disclosure may be used to treat infectious diseases whose clearance is dependent on T cells. Specifically, the method of the disclosure improves T cell function against infectious diseases whose clearance is dependent on T cells.

In still another aspect, the disclosure provides a method to reduce tumor growth in a subject. The method comprises administering to the subject a therapeutic composition comprising isolated T cells that have been cultured in the presence of a composition comprising one or more compounds to promote mitochondrial fusion. The inventors have shown that culturing the T cells in the presence of a composition comprising one or more compounds to promote mitochondrial fusion promotes the longevity of the T cells and enhances cytokine expression. Accordingly, a composition comprising isolated T cells of the disclosure may be used in treating, stabilizing and preventing cancer and associated diseases in a subject. By “treating, stabilizing, or preventing cancer” is meant causing a reduction in the size of a tumor or in the number of cancer cells, slowing or preventing an increase in the size of a tumor or cancer cell proliferation, increasing the disease-free survival time between the disappearance of a tumor or other cancer and its reappearance, preventing an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay (e.g., caspase assays, TUNEL and DNA fragmentation assays, cell permeability assays, and Annexin V assays). Desirably, the decrease in the number of tumor or cancerous cells induced by administration of a T cell of the disclosure is at least 2, 5, 10, 20, or 50-fold greater than the decrease in the number of non-tumor or non-cancerous cells. Desirably, the methods of the present disclosure result in a decrease of 20, 30, 40, 50, 60, 70, 80, 90 or 100% in the size of a tumor or in the number of cancerous cells, as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after at least 5, 10, 15, or 20 years.

In yet another aspect, the present disclosure provides a method to improve vaccination strategies. T cells of the disclosure may enhance the activity of vaccines. Vaccines may be vaccines against infection or cancer. Vaccine compositions comprising T cells of the disclosure may create long-lived immune cells. The longevity of the immune cells may lengthen the duration of protection of the vaccine.

In some embodiments, administration of a composition comprising one or more compounds to promote mitochondrial fusion and/or mitochondrial structural remodeling may be used to treat diseases or disorders associated with mitochondrial dysfunction. For example, neurological disorders such as Parkinson's disease, Alzheimer's disease, Huntington's disease and various β-amyloid disorders.

As used herein, “subject” or “patient” is used interchangeably. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In a preferred embodiment, the subject is human.

### (a) Tumor

T cells of the disclosure may be used to treat a tumor derived from a neoplasm or a cancer. “Neoplasm” is any tissue, or cell thereof, characterized by abnormal growth as a result of excessive cell division. The neoplasm may be malignant or benign, the cancer may be primary or metastatic; the neoplasm or cancer may be early stage or late stage. Non-limiting examples of neoplasms or cancers that may be treated or detected include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In a specific embodiment, the cancer is selected from the group consisting of a leukemia or a lymphoma.

### (b) Administration

T cells of the disclosure may be administered to a subject according to methods known in the art. Such compositions may be administered by any conventional route, including injection or by gradual infusion over time. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS), includes but is not limited to via a lumbar, intraventricular or intraparenchymal catheter or using a surgically implanted controlled release formulation.

The T cells are administered in “effective amounts”, or the amounts that either alone or together with further doses produce the desired therapeutic response (e.g., an immunostimulatory, a cytotoxic response, tumor regression, infection reduction). Actual amount of T cells in a therapeutic composition of the disclosure can be varied so as to administer an amount of T cells that is effective to achieve the desired therapeutic response for a particular subject. The selected amount will depend upon a variety of factors including the activity of the therapeutic composition, formulation, the route of administration, combination with other drugs or treatments, tumor size and longevity, the viral infection, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine. In an aspect, a typical dose contains from about 1×10² to about 1×10⁸ T cells of the disclosure. In an embodiment, a typical dose contains from about 1×10² to about 1×10⁴ T cells of the disclosure. In another embodiment, a typical dose contains from about 1×10³ to about 1×10⁵ T cells of the disclosure. In still another embodiment, a typical dose contains from about 1×10⁴ to about 1×10⁶ T cells of the disclosure. In still yet another embodiment, a typical dose contains from about 1×10⁵ to about 1×10⁷ T cells of the disclosure. In certain embodiments, a typical dose contains from about 1×10⁶ to about 1×10⁸ T cells of the disclosure. In a different embodiment a typical dose contains about 1×10⁵, about 5×10⁵, about 1×10⁶, about 5×10⁶, about 1×10⁷, or about 5×10⁷ T cells of the disclosure.

Administered cells of the disclosure may be autologous (“self”) or heterologous/non-autologous (“non-self,” e.g., allogeneic, syngeneic or xenogeneic). Generally, administration of the cells can occur within a short period of time following the culturing of the T cells (e.g., 1, 2, 5, 10, 24, 48 hours, 1 week or 2 weeks after culturing in the presence of a composition comprising one or more compounds to promote mitochondrial fusion) and according to the requirements of each desired treatment regimen.

Administered T cells of the disclosure may be present in the recipient subject at 1 day or more following administration. For example, T cells of the disclosure may be present at 1, 2, 3, 4, 5, 6, 7, 8, 9, 10, 11, or 12 days or more following administration. Additionally, T cells of the disclosure may be present at 1, 2, 3, 4, 5, 6, 7, 8, 9, 10, 11, or 12 weeks or more following administration. Further, T cells of the disclosure may be present at 1, 2, 3, 4, 5, 6, 7, 8, 9, 10, 11, or 12 months or more following administration. Administered T cells may be present as donor-derived T cells. Methods of detecting the presence of donor-derived T cells are known in the art and may include flow cytometry.

The frequency of dosing may be once, twice, three times or more daily or once, twice, three times or more per week or per month, as needed as to effectively treat the symptoms or disease. In certain embodiments, the frequency of dosing may be once, twice or three times daily. For example, a dose may be administered every 24 hours, every 12 hours, or every 8 hours. In other embodiments, the frequency of dosing may be once, twice or three times weekly. For example, a dose may be administered every 2 days, every 3 days or every 4 days. In a different embodiment, the frequency of dosing may be one, twice, three or four times monthly. For example, a dose may be administered every 1 week, every 2 weeks, every 3 weeks or every 4 weeks.

Duration of treatment could range from a single dose administered on a one-time basis to a life-long course of therapeutic treatments. The duration of treatment can and will vary depending on the subject and the cancer or infection to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

The pharmaceutical composition of the present disclosure is administered in a manner compatible with the dosage formulation, and in a therapeutically effective amount, for example intravenously, intraperitoneally, intramuscularly, subcutaneously, and intradermally. It may also be administered by any of the other numerous techniques known to those of skill in the art, see for example the latest edition of Remington's Pharmaceutical Science, the entire teachings of which are incorporated herein by reference. For example, for injections, the pharmaceutical composition of the present disclosure may be formulated in adequate solutions including but not limited to physiologically compatible buffers such as Hank's solution, Ringer's solution, or a physiological saline buffer. The solutions may contain formulatory agents such as suspending, stabilizing, and/or dispersing agents. Alternatively, the pharmaceutical composition of the present disclosure may be in powder form for combination with a suitable vehicle, e.g., sterile pyrogen free water, before use. Further, the composition of the present disclosure may be administered per se or may be applied as an appropriate formulation together with pharmaceutically acceptable carriers, diluents, or excipients that are well known in the art. In addition, other pharmaceutical delivery systems such as liposomes and emulsions that are well known in the art, and a sustained-release system, such as semi-permeable matrices of solid polymers containing a therapeutic agent, may be employed. Various sustained-release materials have been established and are well-known to one skilled in the art. Further, the composition of the present disclosure can be administered alone or together with another therapy conventionally used for the treatment of a disease/condition in which it is desirable to increase the number of T cells.

The method may further comprise administration of agents standard in the art for treating cancer. Such agents may depend on the type and severity of the cancer, as well as the general condition of the patient. Agents for the treatment of cancer consist primarily of radiation, surgery, chemotherapy and/or targeted therapy. Standard treatment algorithms for each cancer may be found via the National Comprehensive Cancer Network (NCCN) guidelines (www.nccn.org/professionals/physician_gls/f_guidelines.asp). Additionally, the method may further comprise administration of agents standard in the art for treating viral infection.

### (c) Screening

The disclosure provides a method (also referred to herein as a “screening assay”) for identifying modulators, i.e., candidate or test compounds or agents (e.g., peptides, peptidomimetics, small molecules or other drugs) which affect mitochondrial dynamics, for example, mitochondrial fission, mitochondrial fusion, and/or cristae remodeling. Compounds that affect mitochondrial dynamics may enhance T cell survival and/or function.

Screening assays may be used to identify molecules that affect mitochondrial dynamics and/or enhance T cell survival and/or function. For example, mitochondrial morphology, including cristae morphology, may be examined visually. A compound that promotes mitochondrial fusion or inhibits mitochondrial fission may result in mitochondria that are morphologically densely packed, elongated and somewhat tubular. A compound that promotes mitochondrial fusion or inhibits mitochondrial fission may also result in increased mitochondrial mass. A compound that promotes mitochondrial fusion or inhibits mitochondrial fission may also result in cristae tightening and close association of ETC complexes in the inner mitochondrial membrane. Alternatively, protein expression of fusion mediators may be examined. A compound that promotes fusion may cause increased expression of Mfn2 and/or Opa1. Further, phosphorylation of fission factors may be examined. A compound that inhibits fission may reduce phosphorylation of Drp1. Additionally, metabolic activity of cells may be measured. A compound that promotes mitochondrial fusion or inhibits mitochondrial fission may increase OXPHOS activity (as measured by O2 consumption rate (OCR, an indicator of OXPHOS) to extracellular acidification rate (ECAR, an indicator of aerobic glycolysis) ratio), spare respiratory capacity (SRC), and metabolic activity (as measured by OCR). Still further, T cell persistence may be measured. A compound that promotes mitochondrial fusion or inhibits mitochondrial fission may increase T cell longevity in culture and/or following transplantation into a subject. In another embodiment, T cell function may be measured. A compound that promotes mitochondrial fusion or inhibits mitochondrial fission may increase T cell's ability to kill pathogen-infected cells or cancer cells and/or reduce tumor volume.

In one embodiment, the disclosure provides assays for screening candidate or test compounds which affect mitochondrial remodeling. The test compounds of the present disclosure can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386390; Devlin (1990) Science 249:404-406; Cwirla et al. (1990) Proc. Natl. Acad. Sci. 87:6378-6382; and Felici (1991) J. Mol. Biol. 222:301-310).

In one embodiment, an assay is one in which cells are contacted with a test compound and the ability of the test compound to affect mitochondrial remodeling is determined. Determining the ability of the test compound to affect mitochondrial remodeling may be accomplished, for example, by detecting mitochondrial morphology, including cristae morphology. Numerous methods for detecting morphology are known in the art and are contemplated according to the disclosure. Specifically, immunofluorescence or electron microscopy may be used to detect mitochondrial morphology, including cristae morphology. Alternatively, determining the ability of the test compound to affect mitochondrial remodeling may be accomplished, for example, by measuring protein expression of fusion mediators or protein phosphorylation of fission factors. Numerous methods for detecting protein are known in the art and are contemplated according to the disclosure. Specifically, an immunoblot may be used to detect protein expression or phosphorylation. Alternatively, determining the ability of the test compound to affect mitochondrial remodeling may be accomplished, for example, by measuring metabolic activity of cells. Methods of measuring metabolic activity of cells are known in the art. Another method for determining the ability of the test compound to affect mitochondrial remodeling may be accomplished by in vivo experiments. For example, T cell longevity and cytotoxicity may be measured.

In certain embodiments, confocal or electron microscopy is used to visualize changes in mitochondrial structure and dynamics upon contact of a cell with a test compound. Microscopy could be used to measure key morphological parameters including length, total area, networked or rounding scores, cristae density, and/or cristae to area ratios. Following contact of a cell with a compound, other parameters may be examined as correlates of mitochondrial morphology. For example, measurement of cellular and mitochondrial oxygen consumption and production of CO2, lactate, or extracellular acidification rates may be correlated with mitochondrial morphology. In still other embodiments, mitochondrial functions including measurement of membrane potential, ion flux, ATP, NAD/NADH or FAD/FADH₂ ratios and production of reactive oxygen species may be correlated with mitochondrial morphology. In different embodiments, mitochondrial tethering/attachment to cytoskeletal components or cellular organelles may be correlated with mitochondrial morphology. In some embodiments, mitochondrial proximity to other cellular organelles or localization within cells may be correlated with mitochondrial morphology. In various embodiments, mitochondrial protein accessibility by digitonin disruption (as described in the Example), especially as it pertains to ETC supercomplexes, may be correlated with mitochondrial morphology. Further, in vitro or in vivo assays that assess survival and functional capabilities of T cells (see, for example, the Examples) may be correlated with mitochondrial morphology.

This disclosure further pertains to novel agents identified by the above-described screening assays and uses thereof for treatments as described herein.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to the Examples

T cells are important mediators of protective immunity against pathogens and cancer and have several unique properties, not least of which is their ability to proliferate at a rate arguably unlike any other cell in an adult organism. In this regard, one naïve T cell can clonally expand into millions of ‘armed’ T_{E} cells in just a few days (Williams and Bevan, 2007). Concomitant with T cell activation is the engagement of Warburg metabolism, a metabolic phenotype shared by cancer cells and unicellular organisms (Fox et al., 2005, Vander Heiden et al., 2009). Once the source of antigen is cleared, most antigen-specific cells die, but a subset of long-lived, resting T_{M} cells persists (Kaech et al., 2003). T_{M} cells have a unique metabolism that renders them equipped to rapidly respond should infection or tumor growth recur (Pearce et al., 2013). These extensive changes in phenotype and function of T cells go hand in hand with a highly dynamic metabolic range (Maclver et al., 2013, Buck et al., 2015). As such, these cells represent a distinctive and amenable system in which we can study marked changes in cellular metabolism that occur as part of normal cellular development, and not as a result of transformation.

Both OXPHOS and aerobic glycolysis generate energy in the form of ATP, but importantly, are also critical for other essential processes such as the building of biosynthetic precursors for biomass, the production of reactive oxygen species (ROS), and the balance of reducing/oxidizing equivalents like NADH/NAD⁺ which take part in redox reactions that release energy from nutrients. Naïve T (T_{N}) cells use OXPHOS for their metabolic needs, but both OXPHOS and aerobic glycolysis are augmented upon activation (Chang et al., 2013, Sena et al., 2013). The latter is characterized by the preferential conversion of pyruvate to lactate in the cytoplasm rather than its oxidation in the TCA cycle. T_{M} cells predominantly use OXPHOS like T_{N} cells, but have enhanced mitochondrial capacity that is marked by their reliance on FAO to fuel OXPHOS (van der Windt et al., 2012, van der Windt et al., 2013). Failure to engage specific metabolic programs impairs the function and differentiation of T cells (Pearce and Pearce, 2013). Establishing the precise reasons why, and how, these and other cells emphasize one particular metabolic pathway over another remains a challenging prospect.

Mitochondria are essential hubs of metabolic activity, antiviral responses, and cell death (Nunnari and Suomalainen, 2012). These dynamic organelles constantly remodel their structure through fission and fusion events mediated by highly conserved nuclear encoded GTPases (Youle and van der Bliek, 2012, Ishihara et al., 2013). Mitochondrial fission generates smaller, discrete and fragmented mitochondria that can increase ROS production (Yu et al., 2006), facilitate mitophagy (Frank et al., 2012), accelerate cell proliferation (Taguchi et al., 2007, Marsboom et al., 2012), and mediate apoptosis (Youle and Karbowski, 2005). Dynamin-related protein 1 (Drp1) is a cytosolic protein that translocates to the outer mitochondrial membrane (OMM) upon phosphorylation to scission mitochondria (Labrousse et al., 1999, Ingerman et al., 2005, Wakabayashi et al., 2009). Fusion of mitochondria into linear or tubular networks limits deleterious mutations in mitochondrial DNA (mtDNA) (Santel et al., 2003), induces supercomplexes of the ETC maximizing OXPHOS activity (Zorzano et al., 2010, Cogliati et al., 2013, Mishra et al., 2014), and enhances endoplasmic reticulum (ER) interactions important for calcium flux (de Brito and Scorrano, 2008). In addition, mitochondria elongate as a survival mechanism in response to nutrient starvation and cell stress, linking fusion to cellular longevity (Gomes et al., 2011, Rambold et al., 2011a, Friedman and Nunnari, 2014). OMM fusion is mediated by mitofusin 1 and 2 (Mfn1, Mfn2) isoforms (Chen et al., 2003), while inner membrane fusion is controlled by optic atrophy 1 (Opa1) protein (Cipolat et al., 2004). Complete organismal deficiency in any of these proteins is embryonically lethal and mutations in the genes that encode them underlie the cause of several human diseases (Chen et al., 2007, Zhang et al., 2011, Chan, 2012, Archer, 2014).

Mitochondrial membrane remodeling has been largely demonstrated to be acutely responsive to changes in cellular metabolism (Mishra and Chan, 2016, Wai and Langer, 2016), but whether it plays a dynamic role in shaping metabolic pathways has been inferred but not extensively studied. At the cellular level, deletion of any of the fission and fusion machinery perturbs OXPHOS and glycolytic rates at baseline (Liesa and Shirihai, 2013). Tissue-specific deletion of Mfn2 in muscles of mice disrupts glucose homeostasis (Sebastian et al., 2012) and Drp1 ablation in the liver results in reduced adiposity and elevated whole-body energy expenditure, protecting mice from diet-induced obesity (Wang et al., 2015). A recent study has also suggested a link between Drp1 mediated fission and its affect on glycolysis during cell transformation (Serasinghe et al., 2015). The central question of whether fission/fusion and associated changes in cristae morphology actively control the adoption of distinct metabolic programs and therefore regulates T cell responses however, remains unanswered.

### Example 1. Unlike T_{E} Cells, T_{M} Cells Maintain a Fused Mitochondrial Network

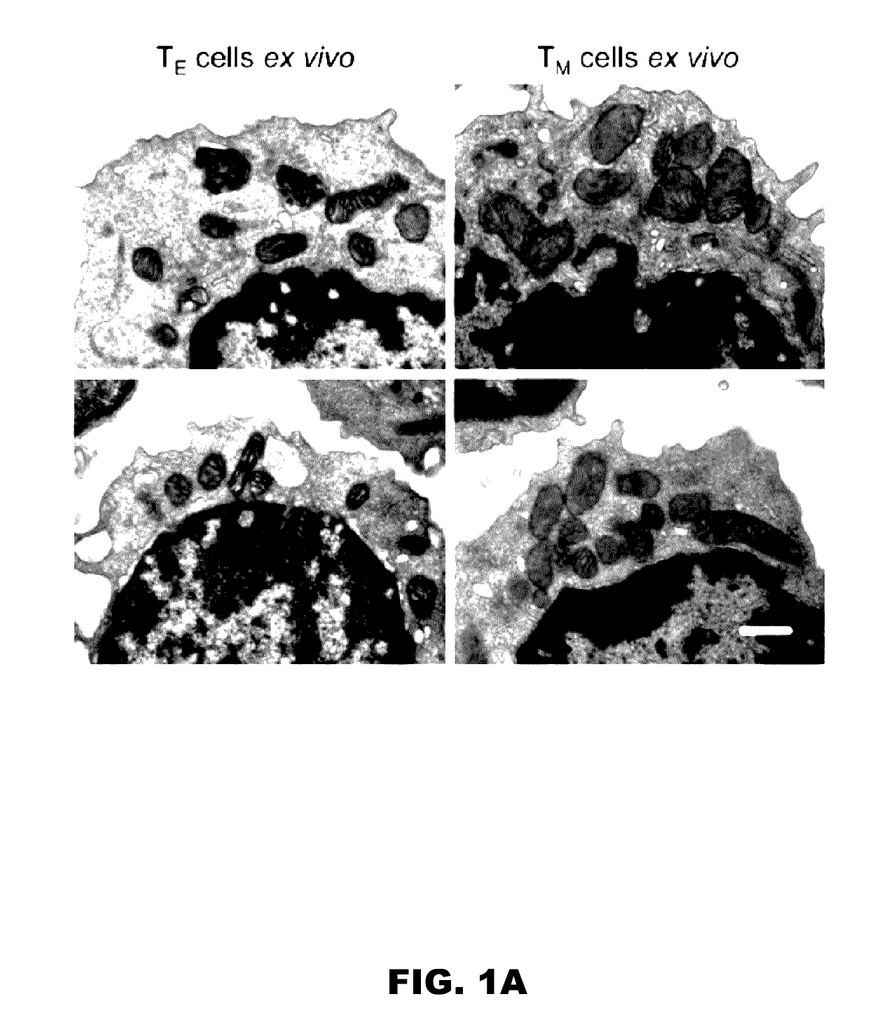

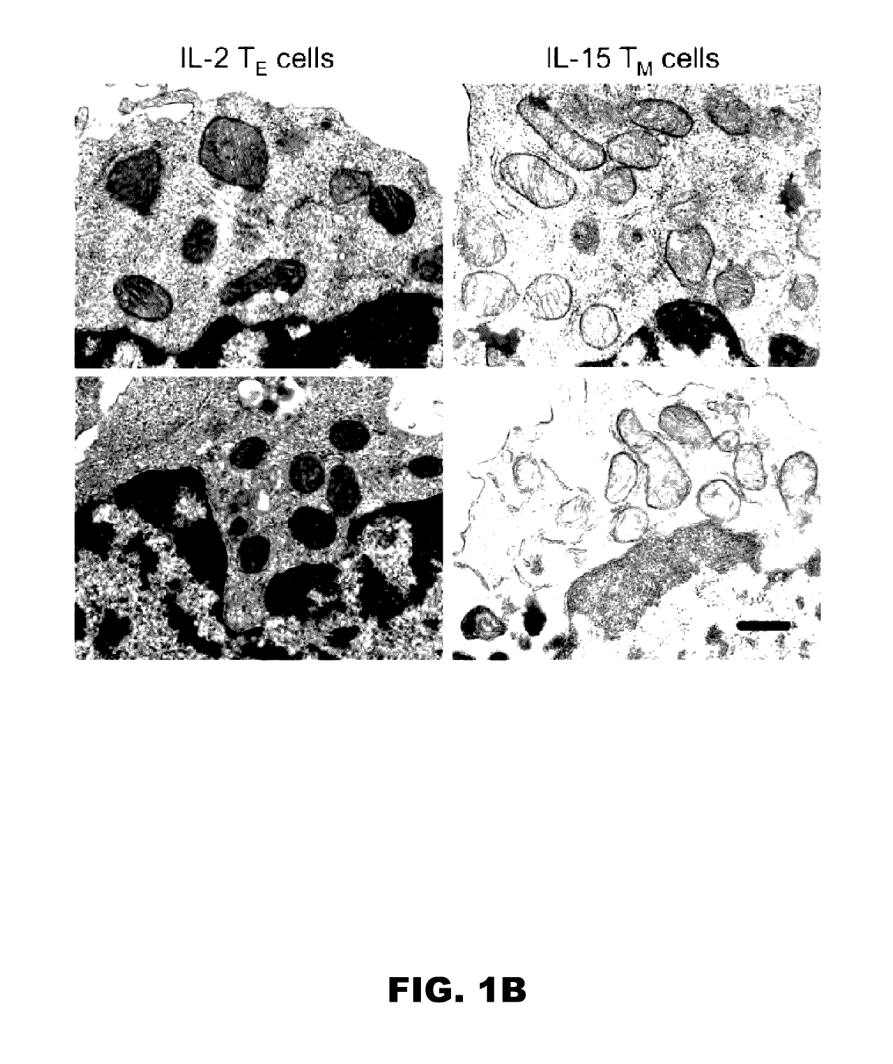

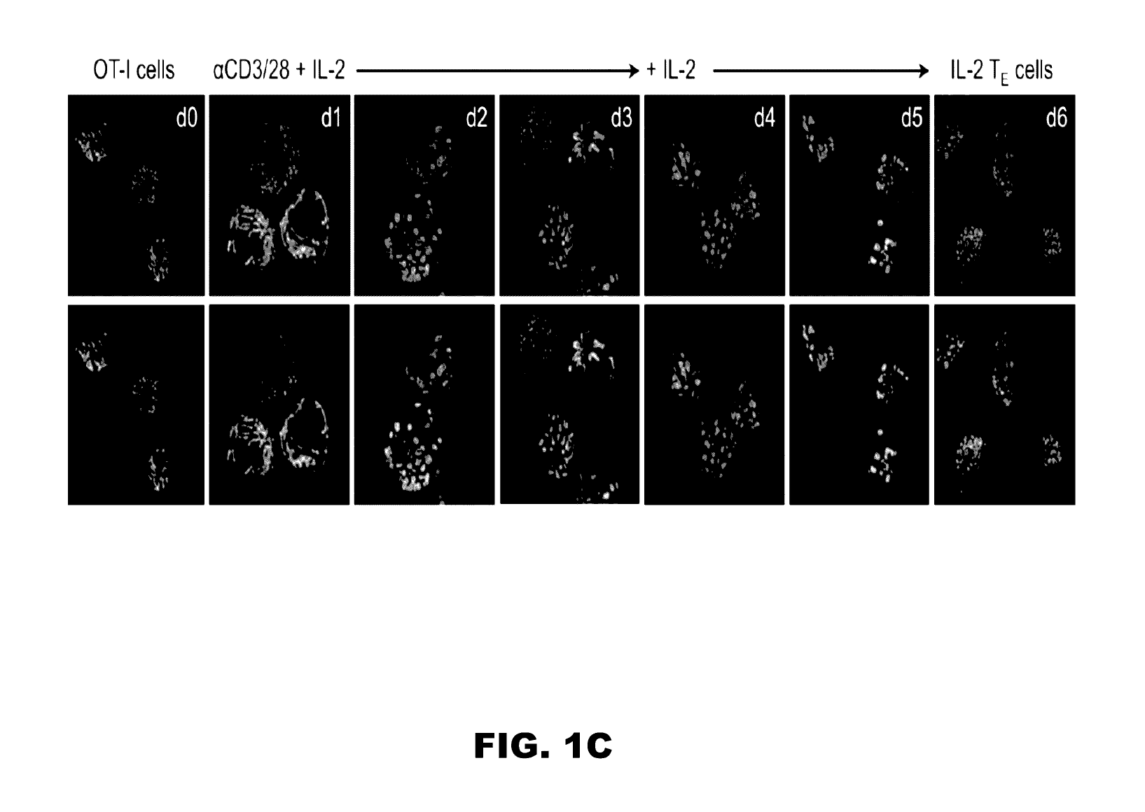

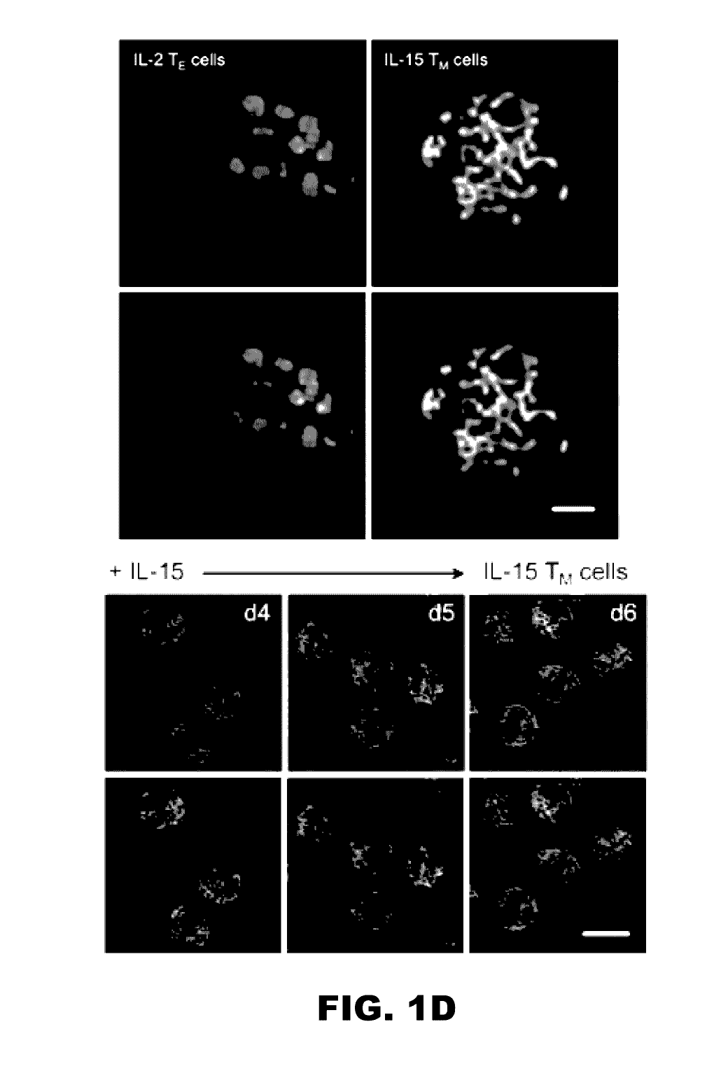

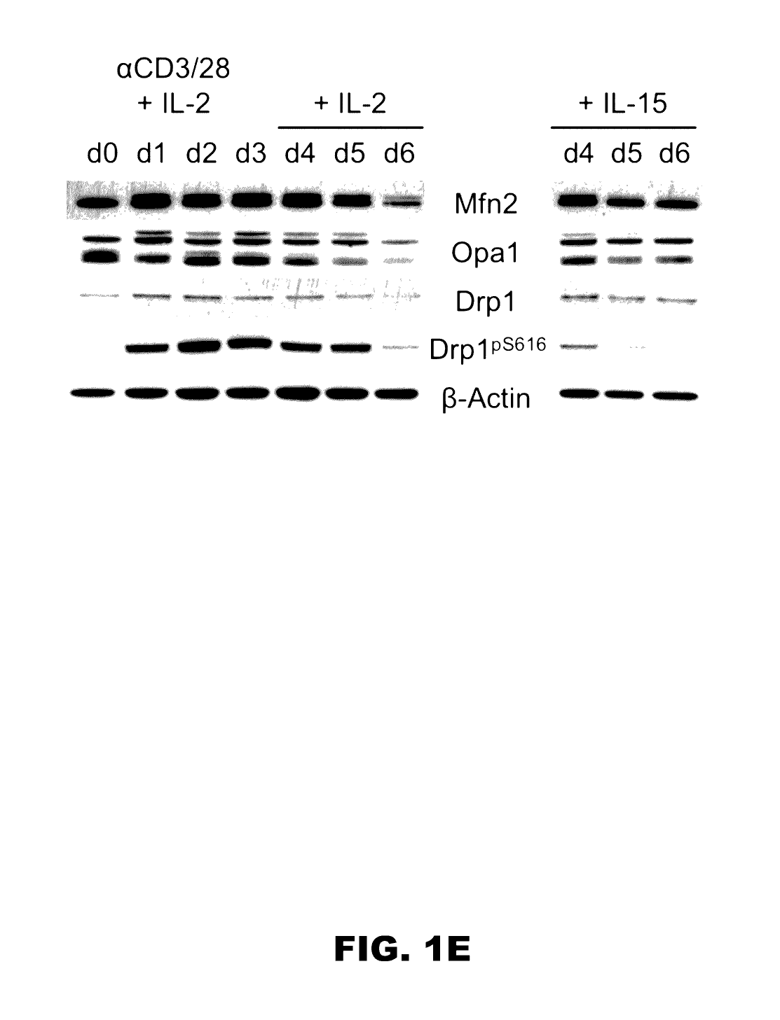

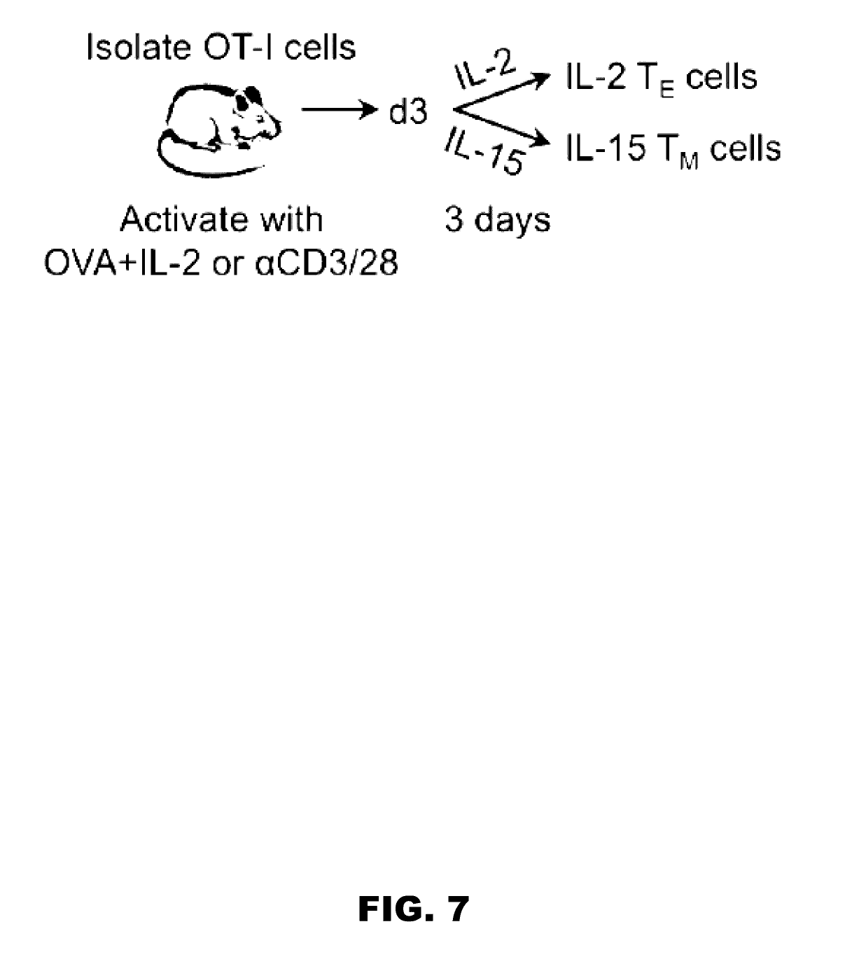

T_{M} cells have more mitochondrial mass than T_{E} or T_{N} cells and suggested that mitochondria in these T cell subsets possess distinct morphologies. These observations prompted us to more closely assess mitochondrial structure in T cells. We infected mice with Listeria monocytogenes expressing ovalbumin (OVA) (LmOVA) and isolated T_{E} and T_{M} cells for ultrastructure analysis by electron microscopy (EM). We found that T_{E} cells had small, distinct mitochondria dispersed in the cytoplasm, while T_{M} cells had more densely packed, somewhat tubular, mitochondria (FIG. 1A). In order to more thoroughly investigate these morphological differences, we differentially cultured activated OVA-specific T cell receptor (TCR) transgenic OT-I cells in interleukin-2 (IL-2) and IL-15 to generate IL-2 T_{E} and IL-15 T_{M} cells (FIG. 7) (Carrio et al., 2004). These culture conditions approximate T cell responses in vivo and allow us to generate large numbers of cells amenable to further experimentation in vitro (O'Sullivan et al., 2014). We found that IL-2 T_{E} and IL-15 T_{M} cells possessed similar mitochondrial ultrastructure as their ex vivo isolated counterparts (FIG. 1B). Next, we acquired live Z-stacked images of these T cells over time by confocal microscopy and found that while at day 1 after activation the mitochondria appeared fused, from days 2-6 after activation, IL-2 T_{E} cells exhibited predominantly punctate mitochondria (FIG. 1C). In contrast, once cells were exposed to IL-15, a cytokine that supports TM cell formation (Schluns et al., 2002), the mitochondria formed elongated tubules (FIG. 1C). Magnified 3D rendered images from these experiments emphasized the marked differences in mitochondrial morphology between the IL-2 T_{E} and IL-15 T_{M} cells (FIG. 1D). Together these data suggest that the mitochondria in T_{E} cells are actively undergoing fission, while in T_{M} cells, these organelles exist in a fused state. To further investigate these changes in mitochondrial morphology, we assessed the expression of several critical protein regulators of mitochondrial dynamics. We found that by day 6, fusion mediators Mfn2 and Opa1 were lower in T_{E} cells compared to T_{M} cells, while fission factor Drp1 was more highly phosphorylated at its activating site Ser616 in T_{E} cells (FIG. 1E) (Marsboom et al., 2012). These data are consistent with our observations that mitochondria in T_{M} cells appear more fused than those in T_{E} cells.

### Example 2. Mitochondrial Inner Membrane Fusion Protein Opa1 is Necessary for T_{M} Cell Generation

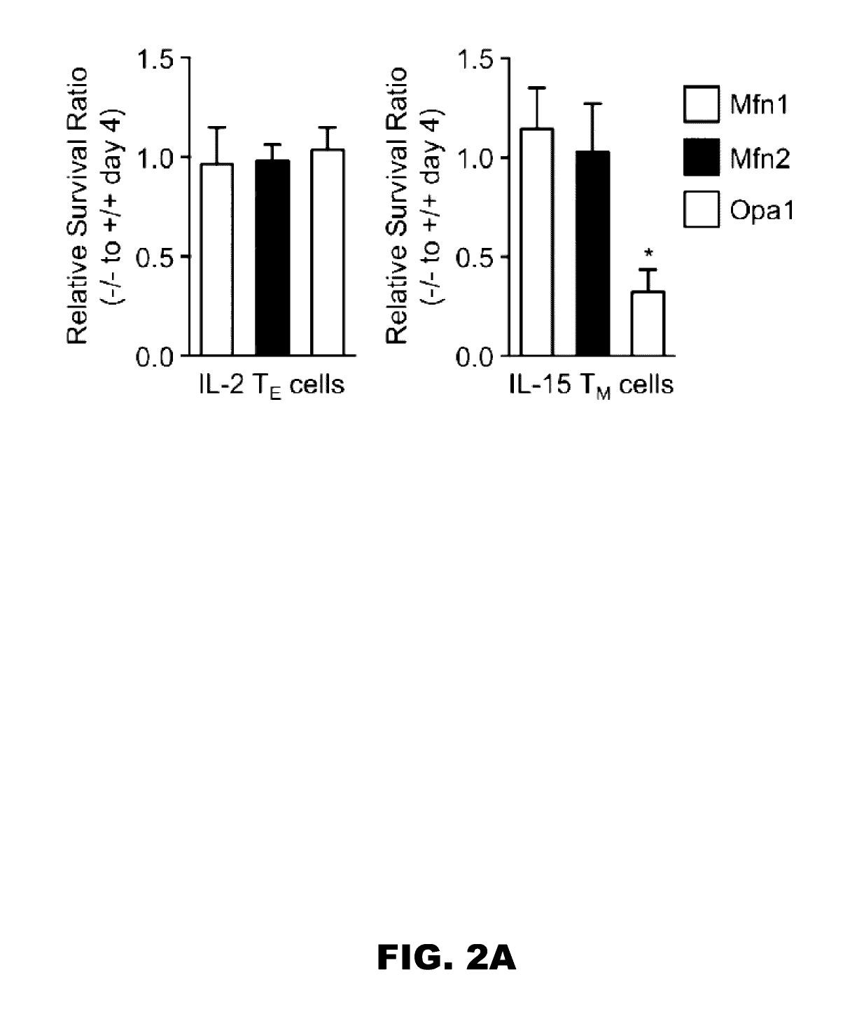

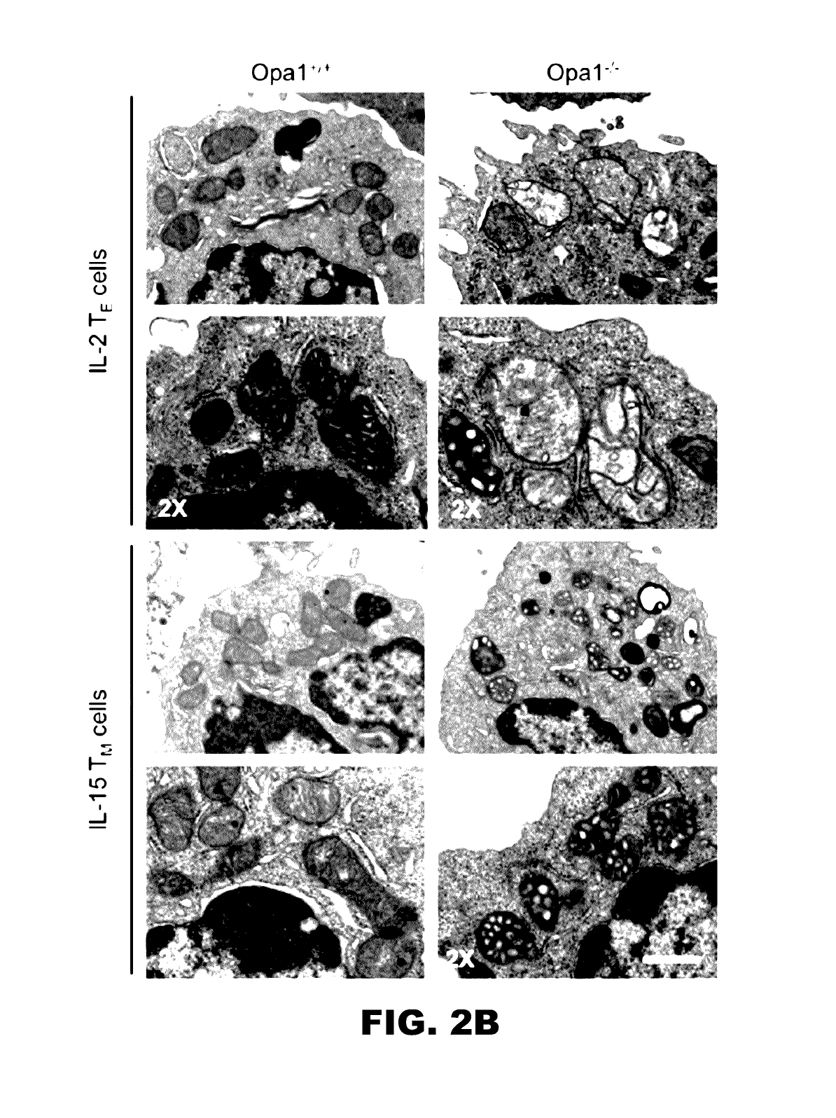

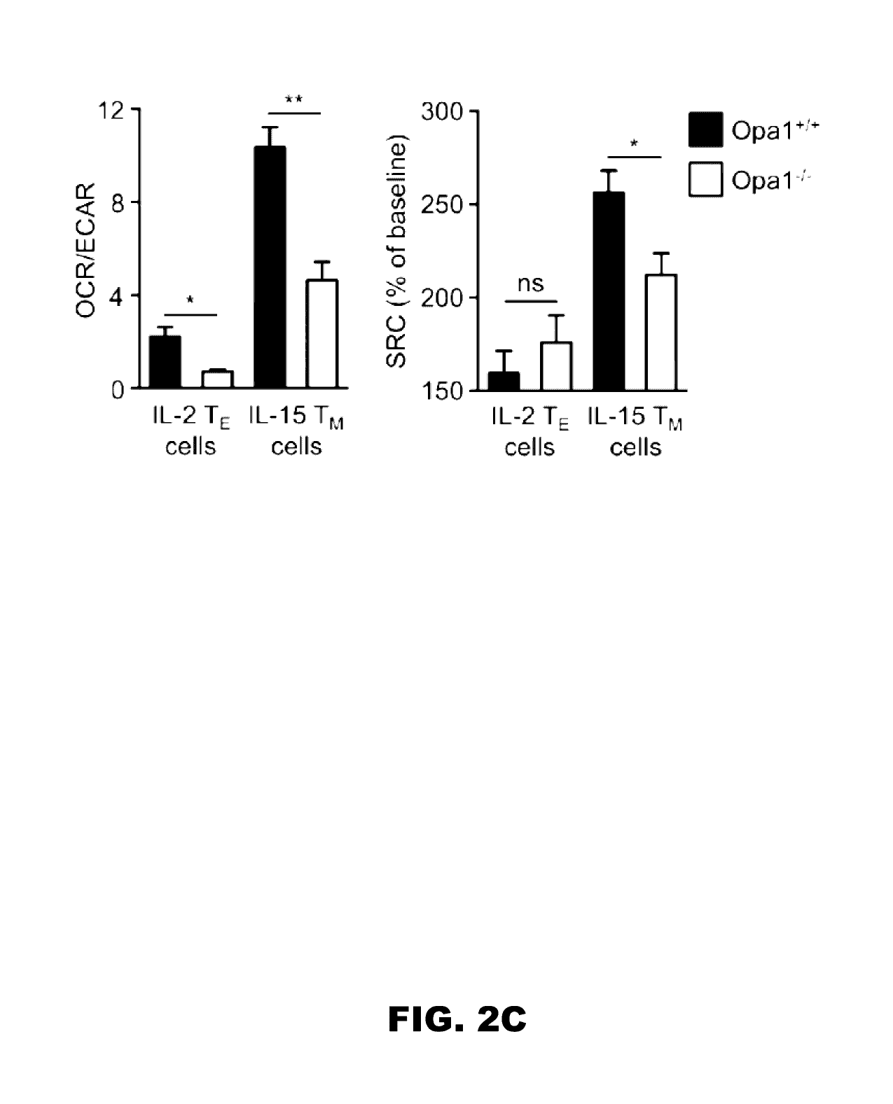

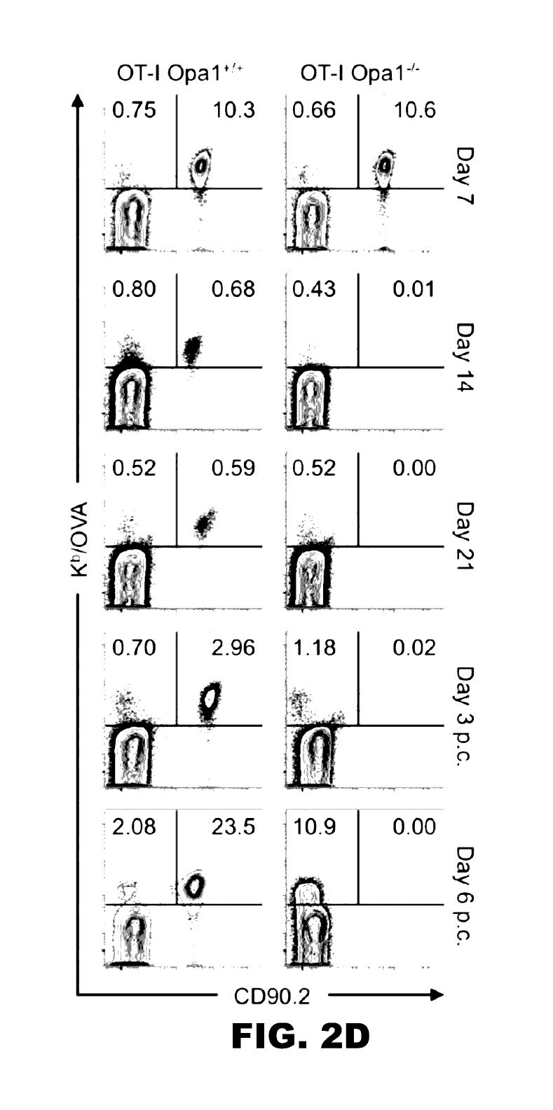

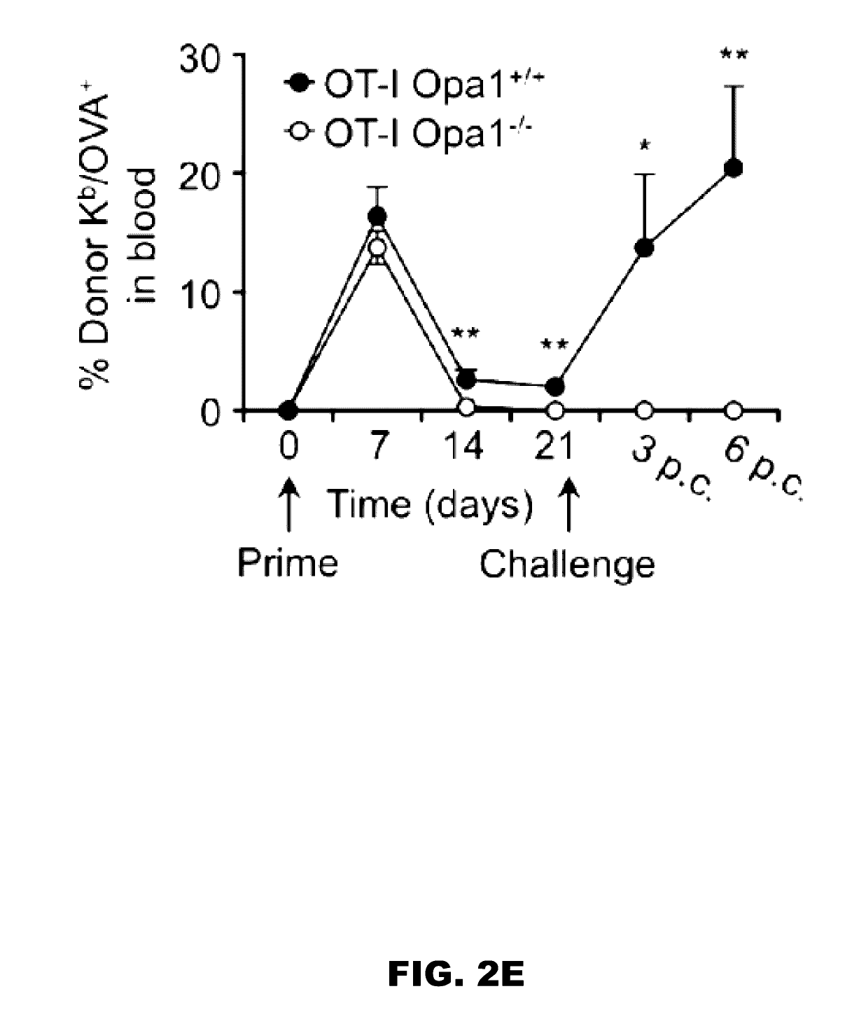

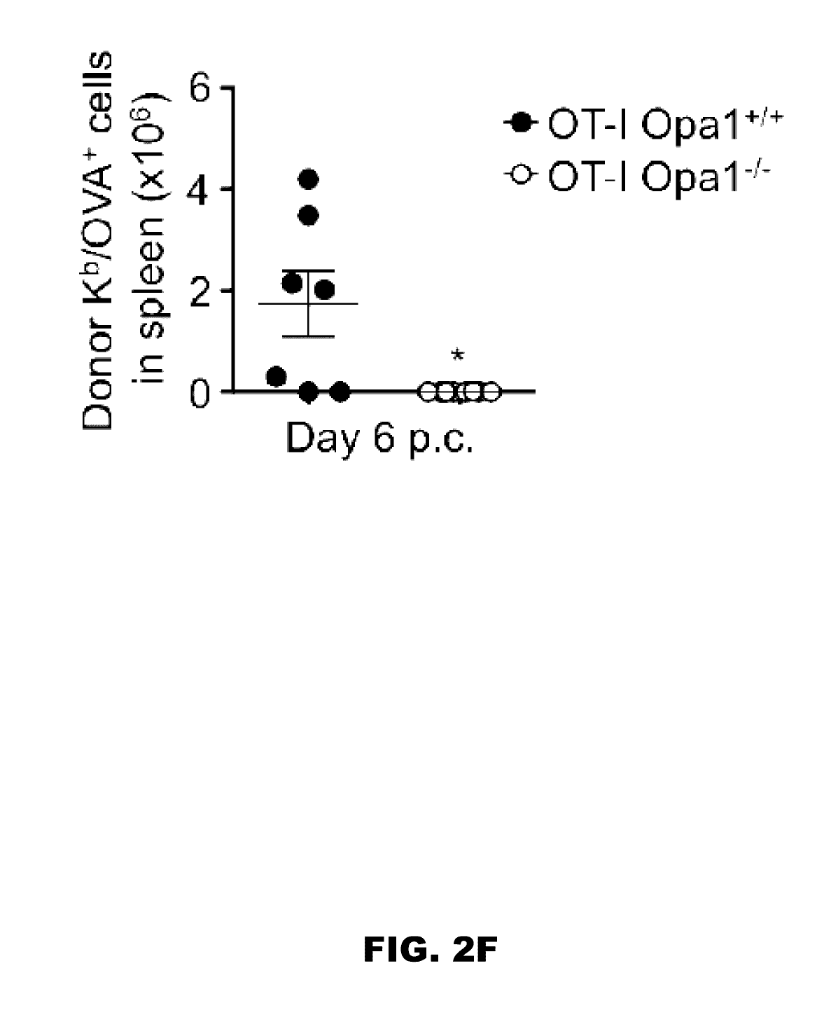

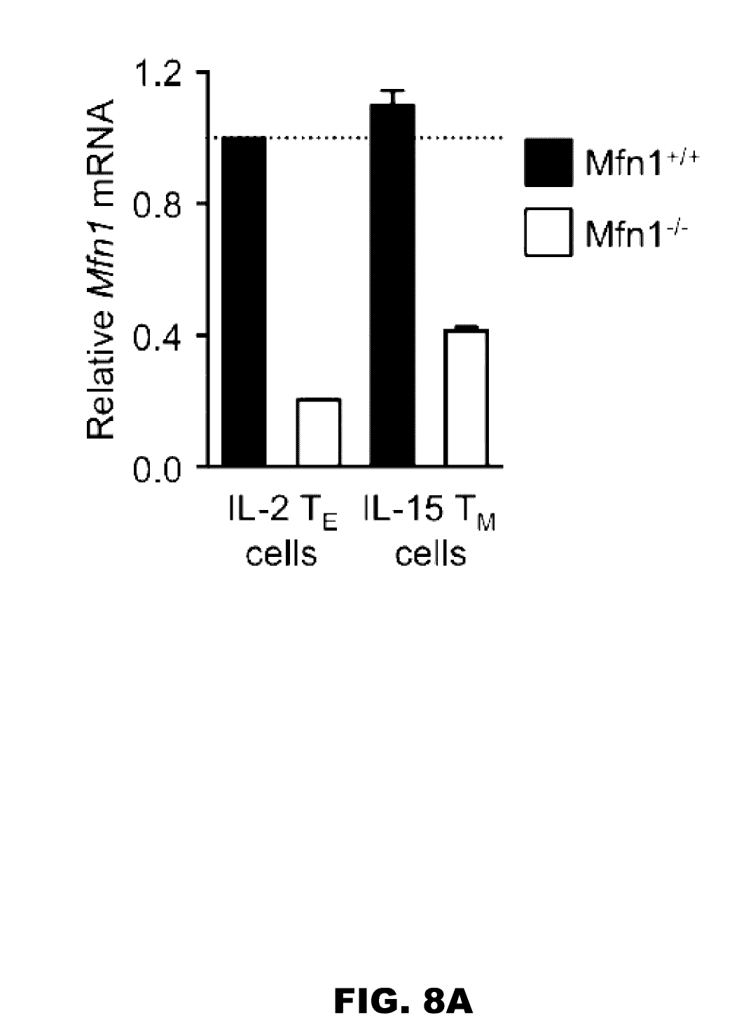

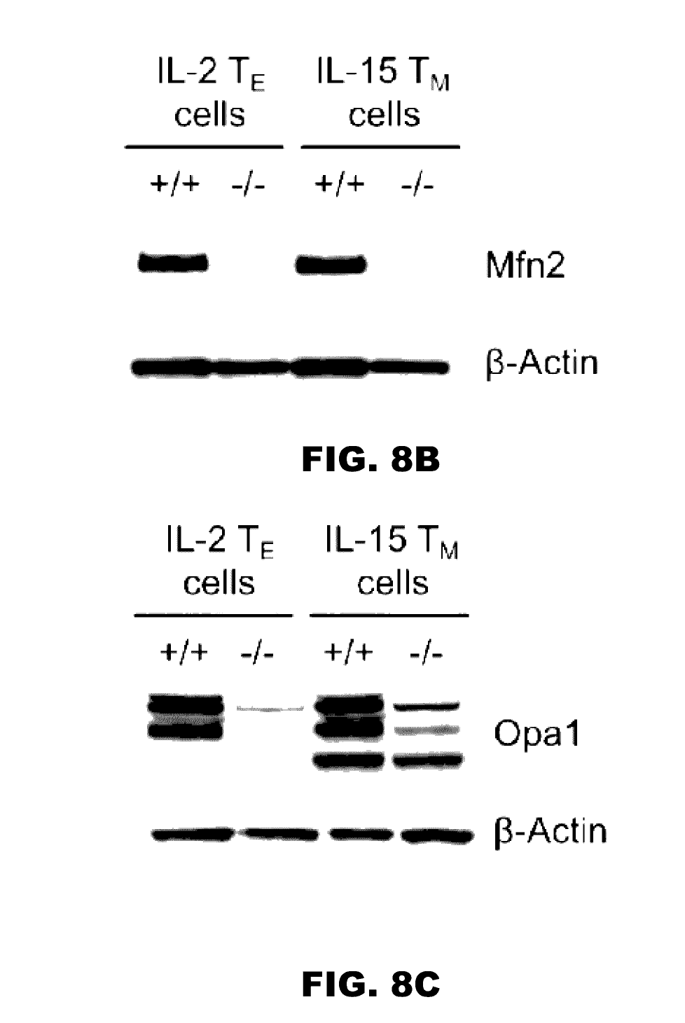

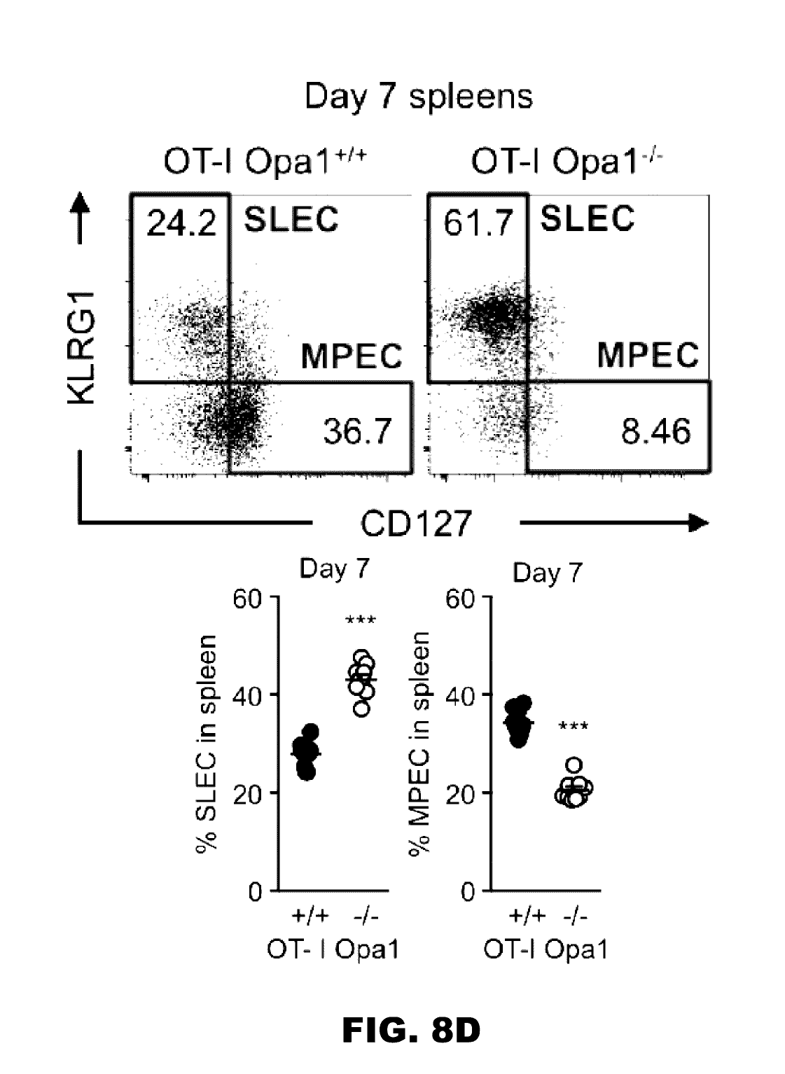

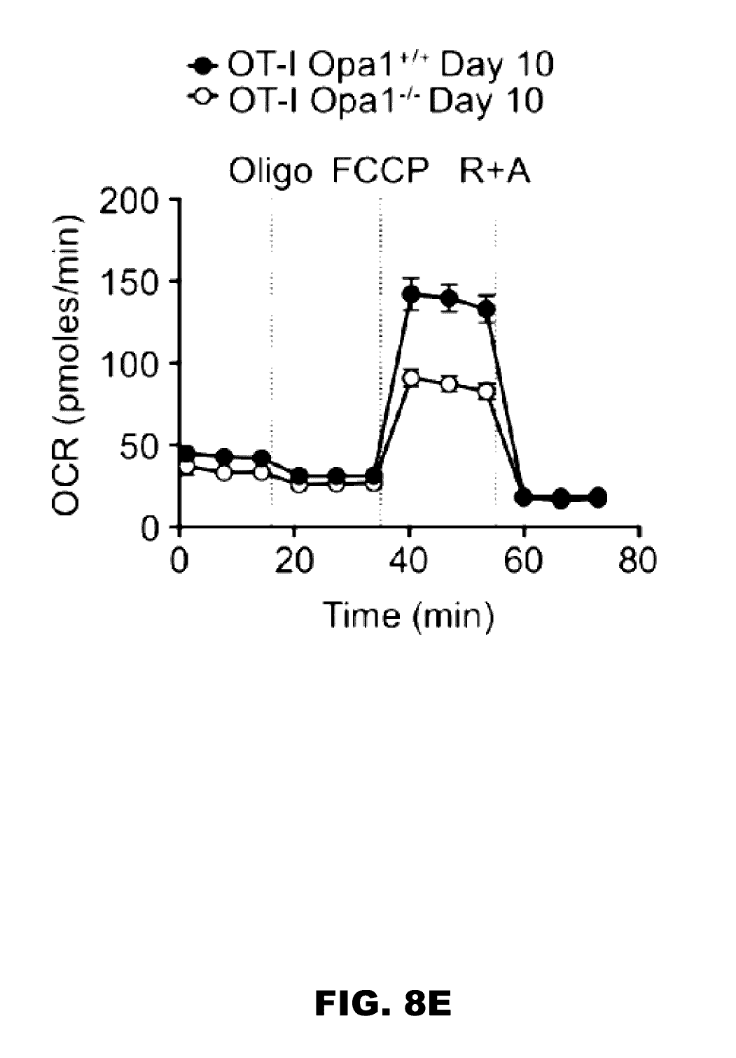

We questioned next whether mitochondrial fusion was important for T_{M} cell generation and survival. We crossed Mfn1, Mfn2, and Opa1 floxed mice to OT-I CD4 Cre transgenic mice to conditionally delete these proteins in T cells. Peripheral T cell frequencies in these mice were grossly normal (data not shown). We differentially cultured these Mfn^{-/-}, Mfn2^{-/-}, and Opa1^{-/-} OT-I T cells in IL-2 and IL-15 and found that only Opa1^{-/-} T cells displayed a selective defect in survival when cultured in IL-15 (FIG. 2A). Opa1 deficiency did not effect IL-2 T_{E} cell survival. We measured the efficiency of gene deletion by mRNA and/or protein analyses (FIG. 8A, FIG. 8B, FIG. 8C). While Mfn1 and 2 were efficiently deleted, we found some residual expression of Opa1 particularly in IL-15 T_{M} cells, suggesting that we were selecting for cells that retained expression of Opa1 in IL-15 culture conditions, albeit at a diminished level as most of these cells die (FIG. 2A). We also assessed mitochondrial ultrastructure and, in agreement with published results for other cell types (Zhang et al., 2011, Cogliati et al., 2013), mitochondrial cristae were significantly altered and disorganized in the absence of Opa1 (FIG. 2B). Consistent with their survival defect, Opa1^{-/-} IL-15 T_{M} cells exhibited decreased OXPHOS activity, as measured by 02 consumption rate (OCR, an indicator of OXPHOS) to extracellular acidification rate (ECAR, an indicator of aerobic glycolysis) ratio, and spare respiratory capacity (SRC), compared to normal cells (FIG. 2C). SRC is the extra mitochondrial capacity available in a cell to produce energy under conditions of increased work or stress and is thought to be important for long-term cellular survival and function (measured as OXPHOS activity above basal after uncoupling with FCCP) (Yadava and Nicholls, 2007, Ferrick et al., 2008, Choi et al., 2009, Nicholls, 2009, Nicholls et al., 2010, van der Windt et al., 2012). To determine whether Opa1 function is required for T_{M} cell development in vivo, we adoptively transferred naïve Opa1^{-/-} OT-I T cells into congenic recipients, infected these mice with LmOVA, and subsequently assessed T_{M} cell formation in the weeks after infection. Control and Opa1^{-/-} OT-I T cells mounted normal T_{E} cell responses (day 7) to infection, while Opa1^{-/-} OT-I T_{M} cell formation (days 14-21) was drastically impaired (FIG. 2D). Consistent with diminished T_{M} cell development, a significantly higher proportion of short-lived effector cells to memory precursor effector cells were present within the Opa1^{-/-} OT-I donor cell population 7 days after infection (FIG. 8D) (Kaech et al., 2003). In addition, at day 10 post-infection, a time point at which T_{E} cells contract, while T_{M} cells emerge, Opa1^{-/-} T cells isolated ex vivo had decreased SRC compared to control cells (FIG. 8E), correlating with their decreased survival. To assess whether Opa1^{-/-} T_{M} cells existed in too low an abundance to be discerned by flow cytometry, we challenged these mice with a second infection. We observed no recall response (day 3 and 6 p.c.) from Opa1^{-/-} T cells when assessing frequency (FIG. 2E) or absolute numbers (FIG. 2F), while there was considerable expansion of control donor cells. These data illustrate that Opa1 function is required for T_{M} cell, but not T_{E} cell generation.

### Example 3. Mitochondrial Fusion Imposes a T_{M} Cell Phenotype, Even in the Presence of Activating Signals

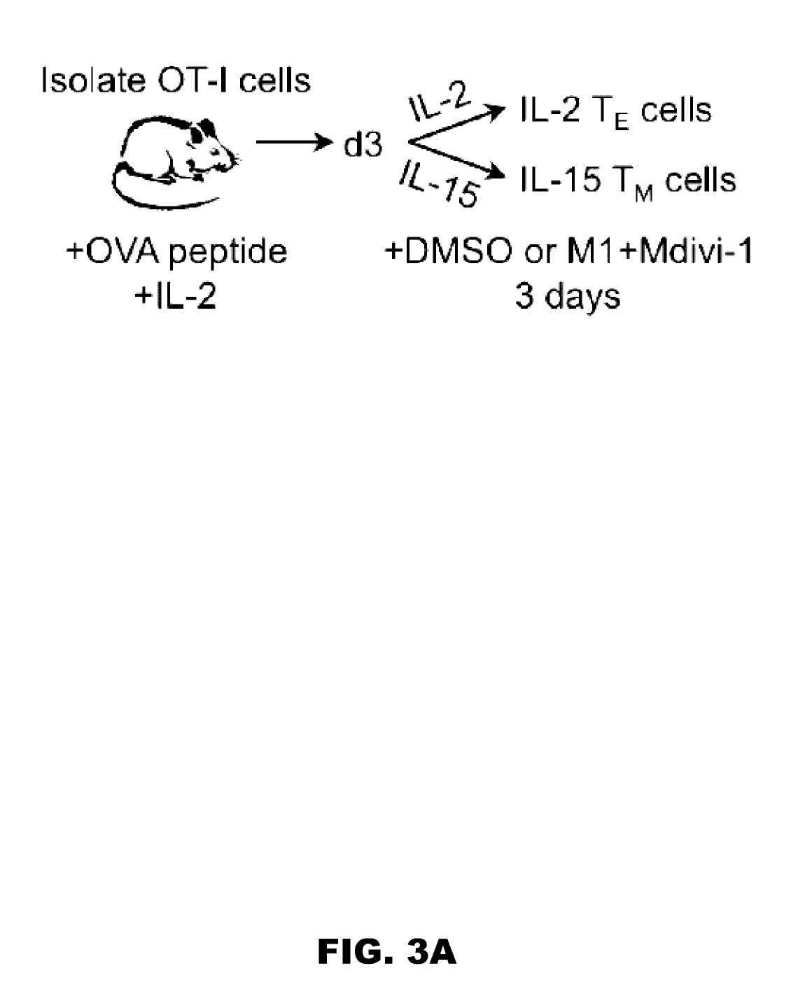

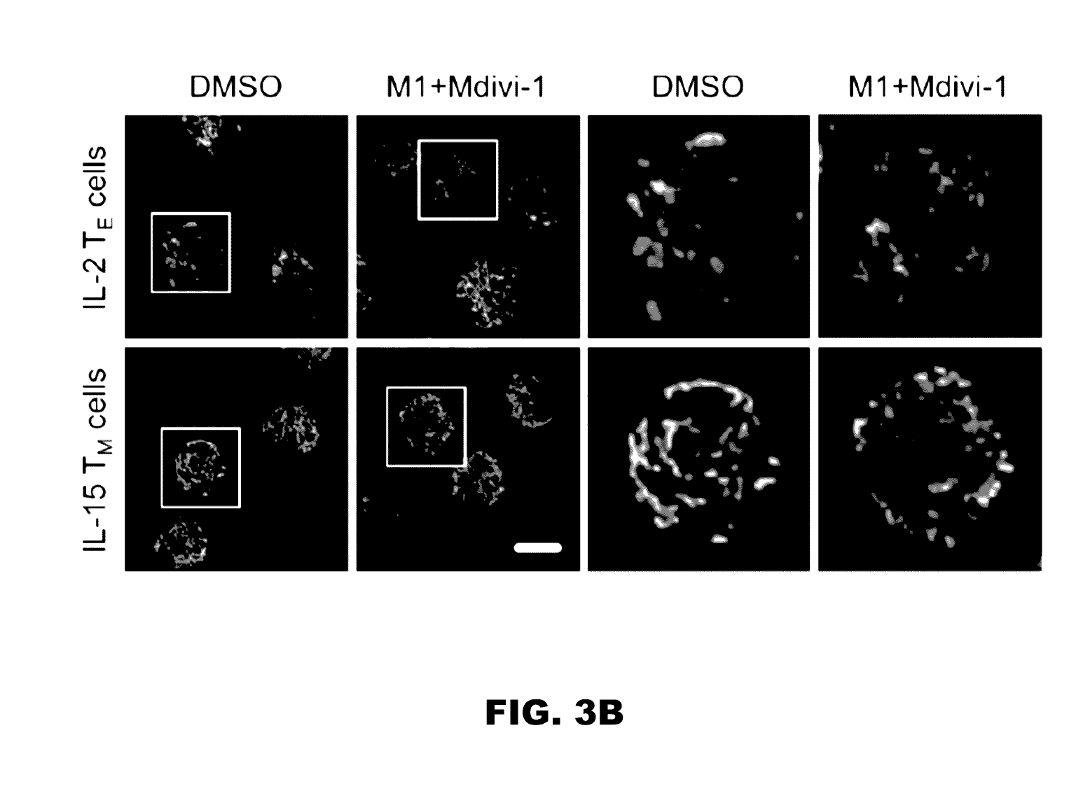

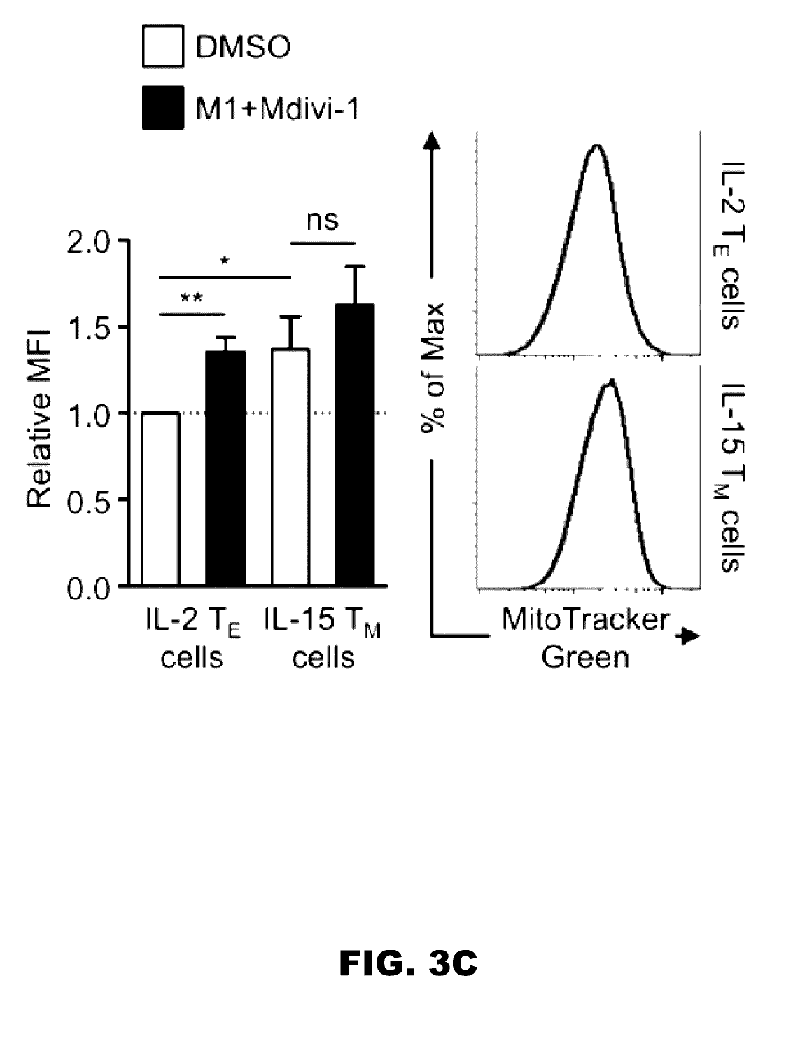

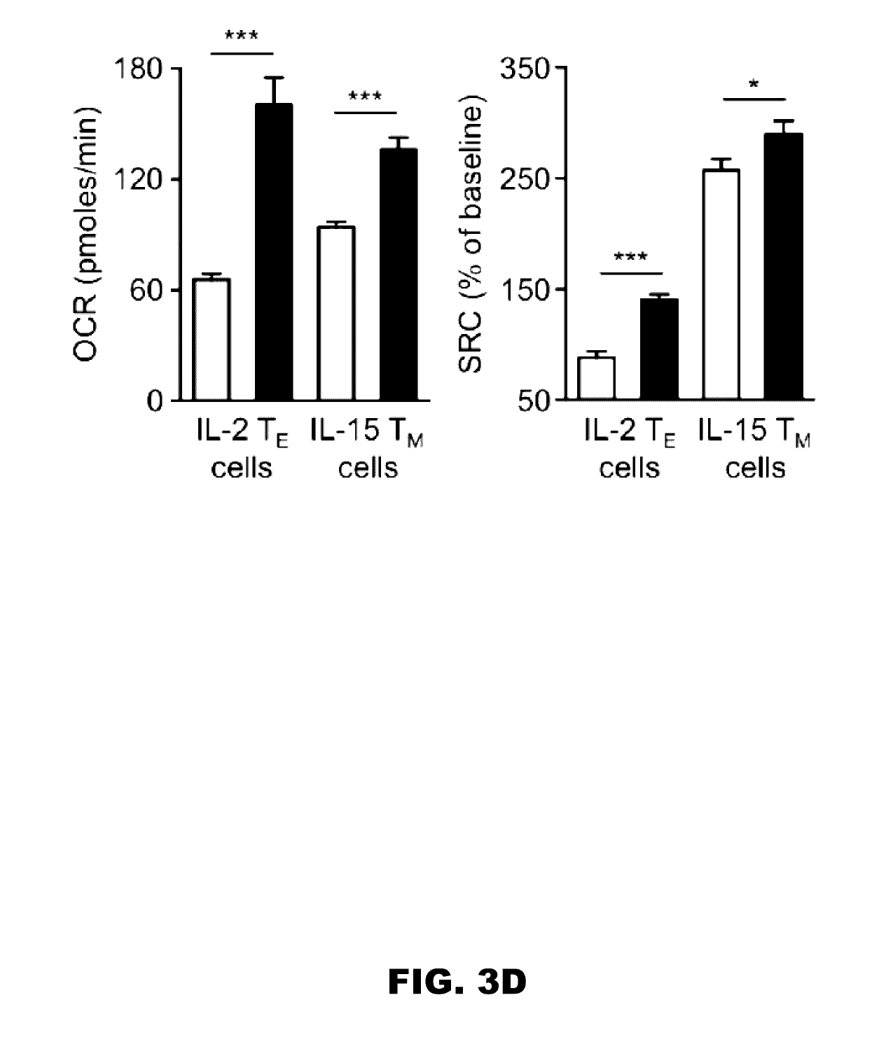

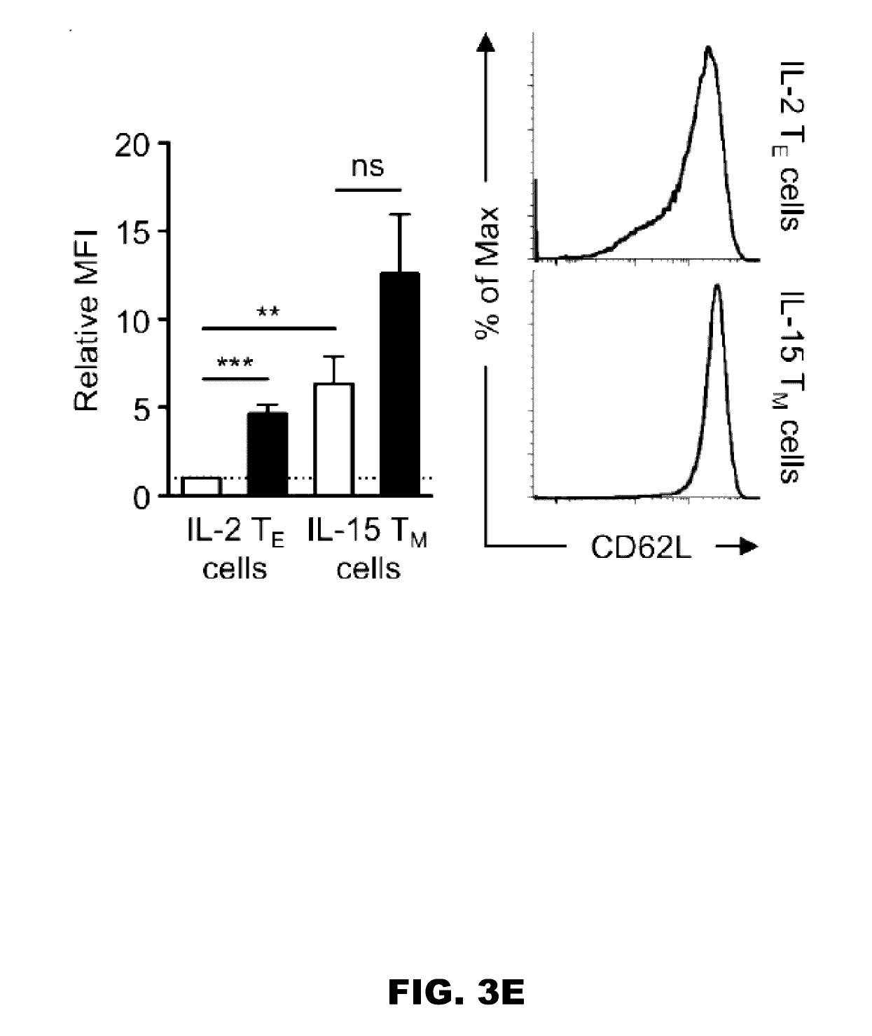

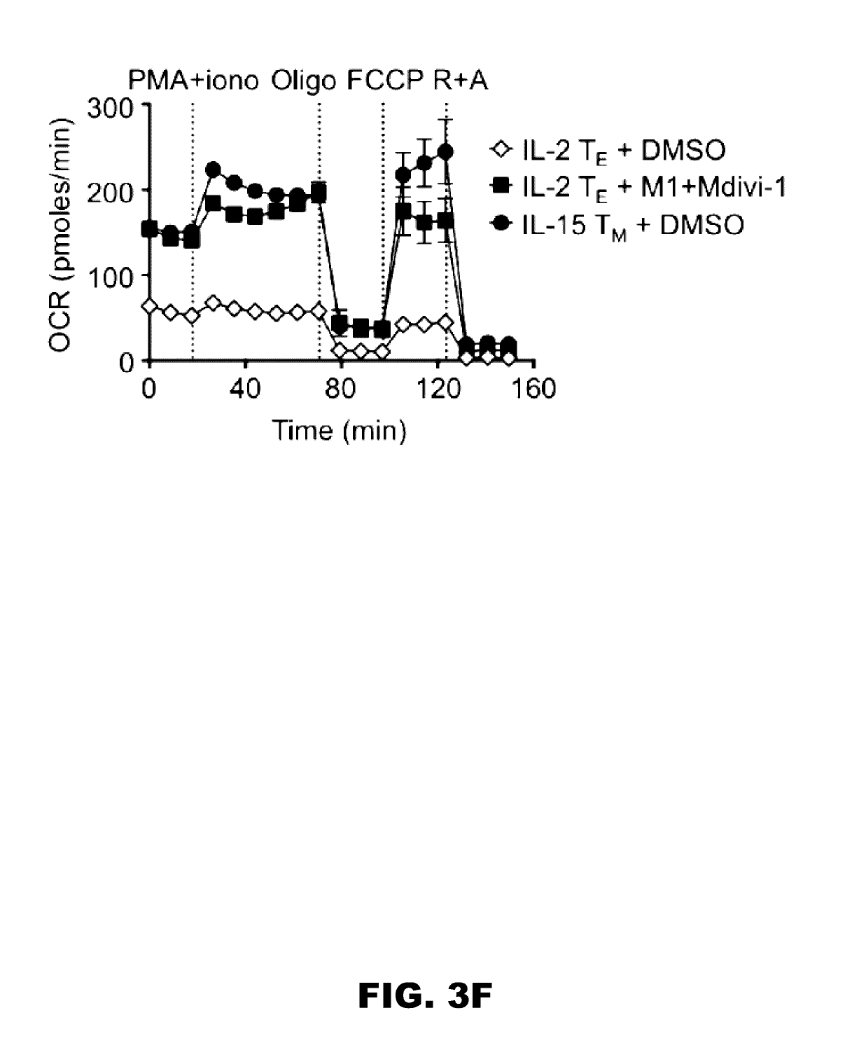

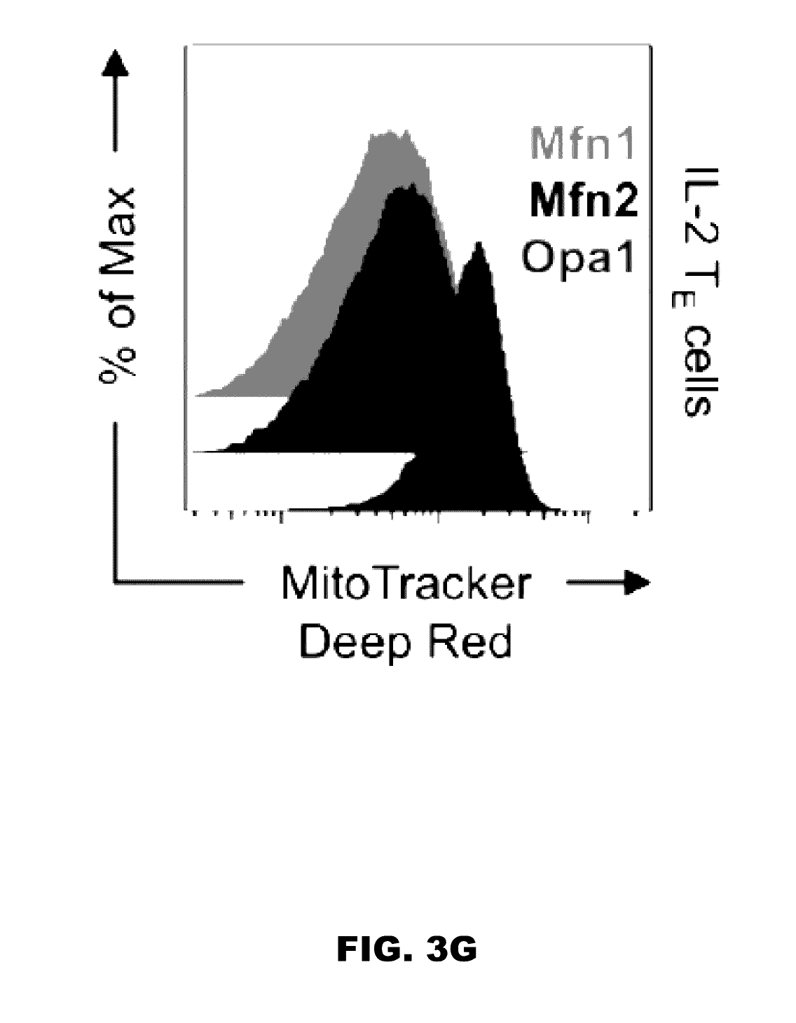

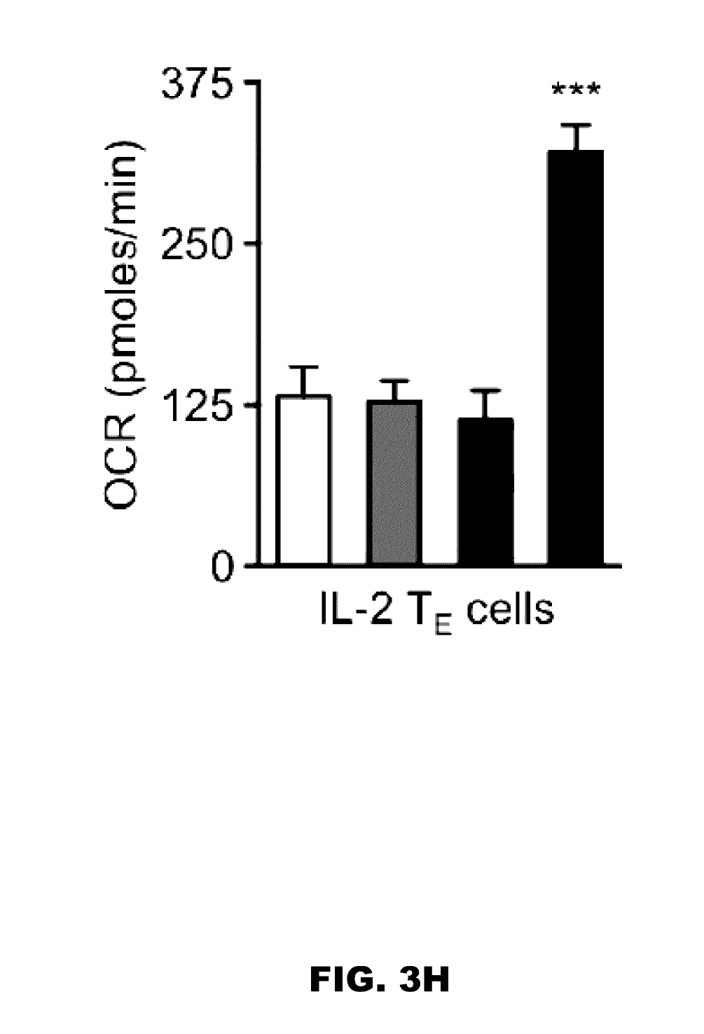

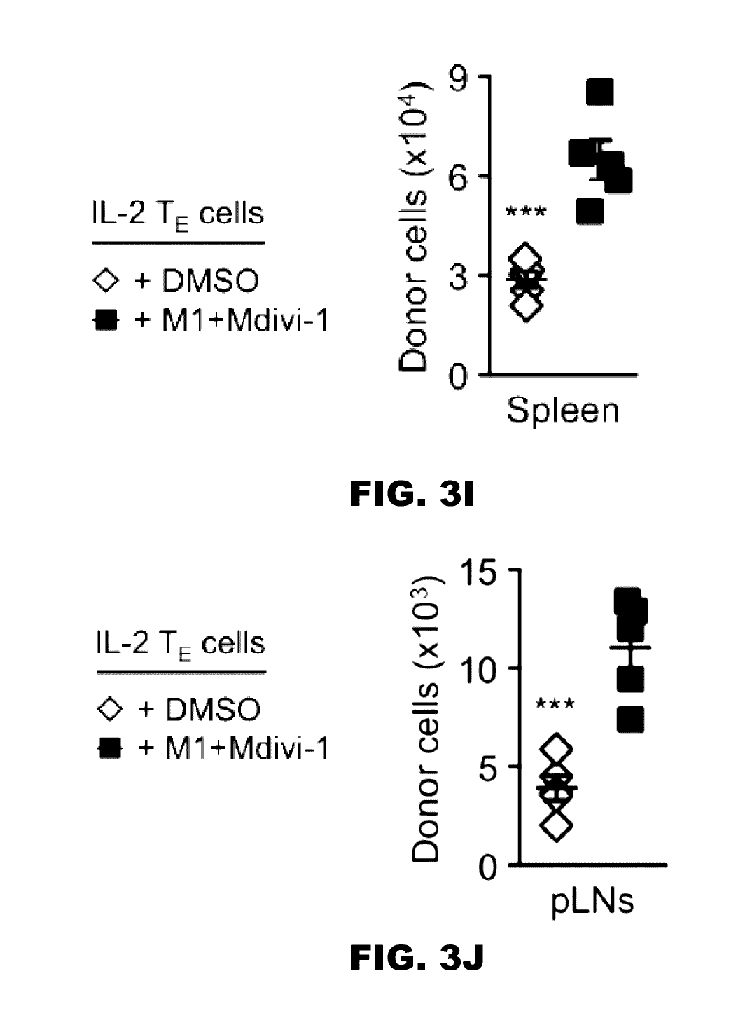

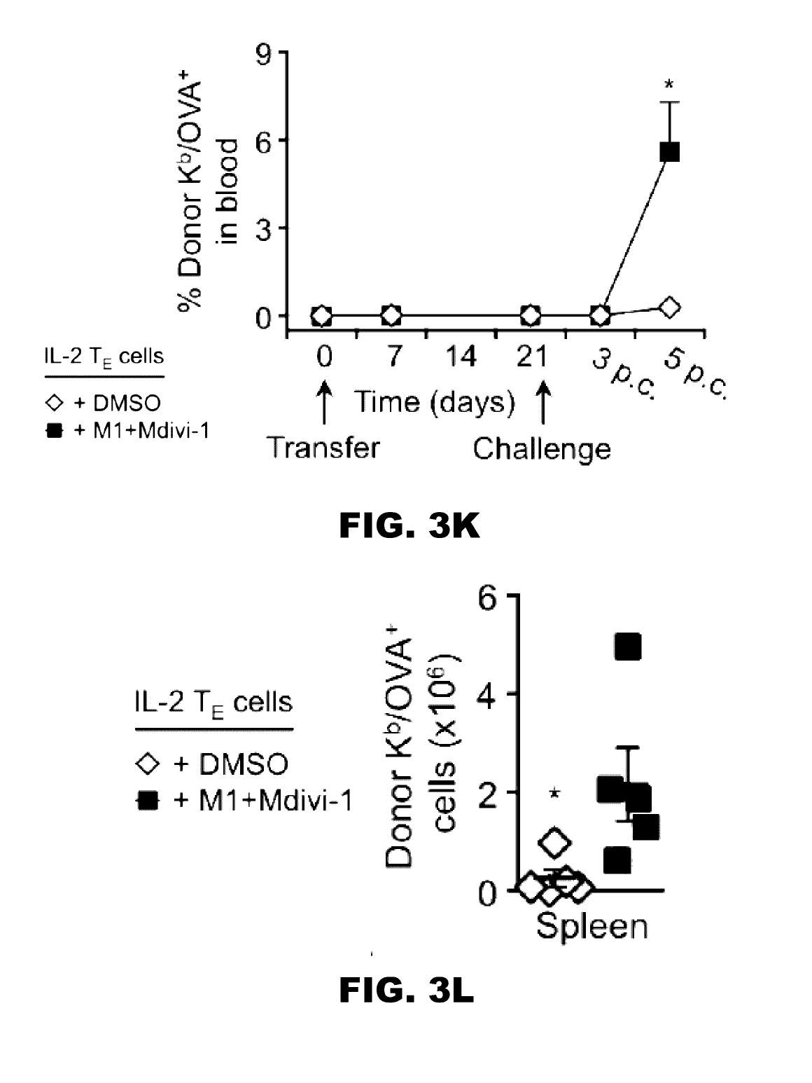

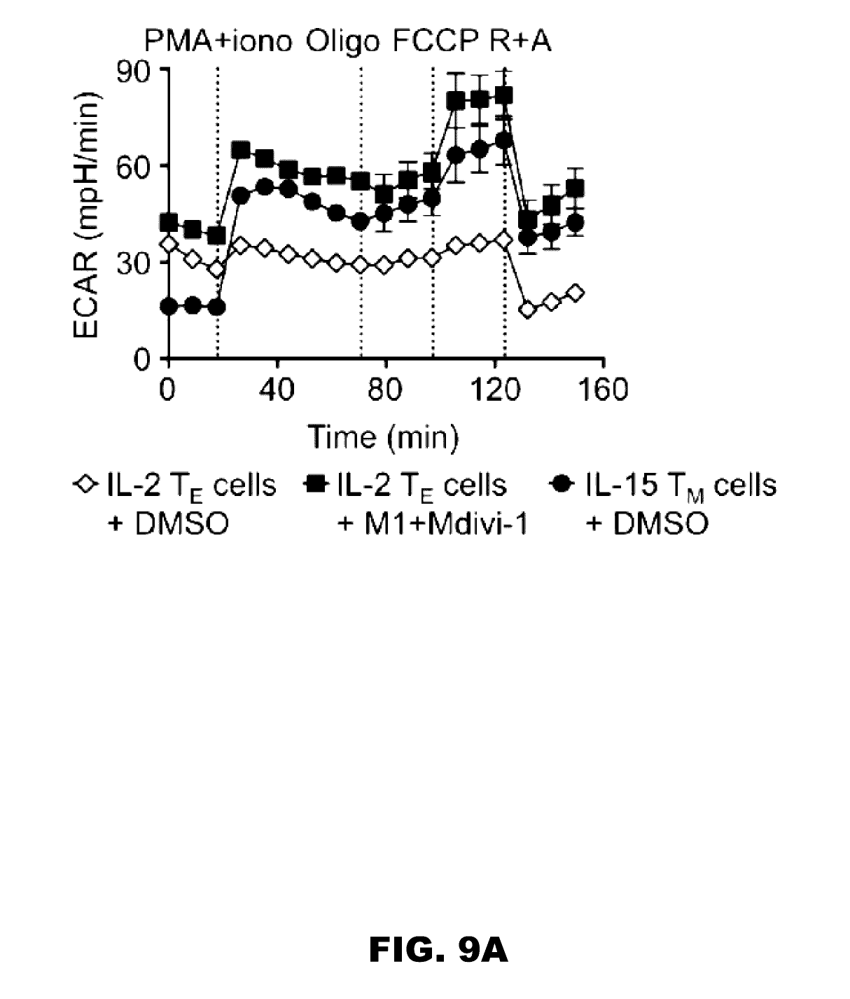

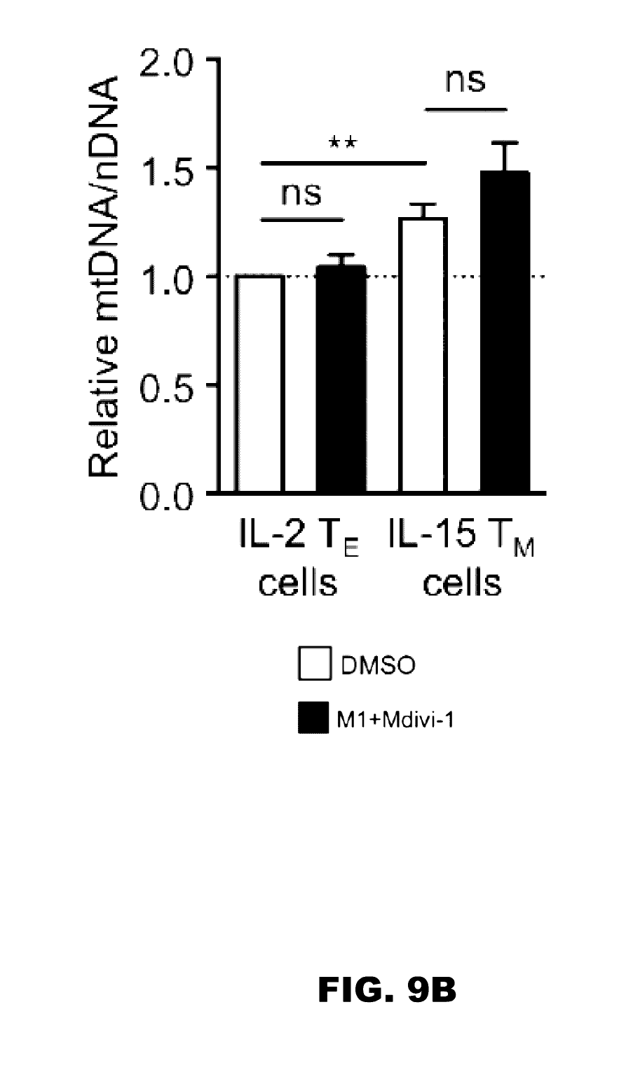

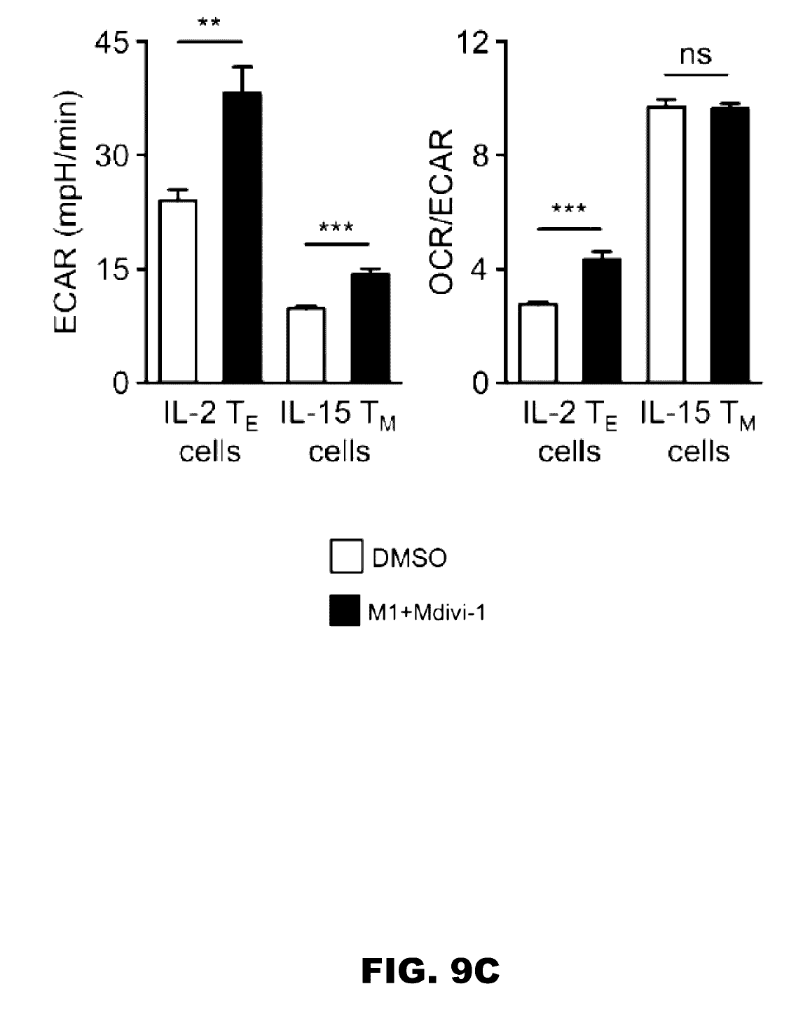

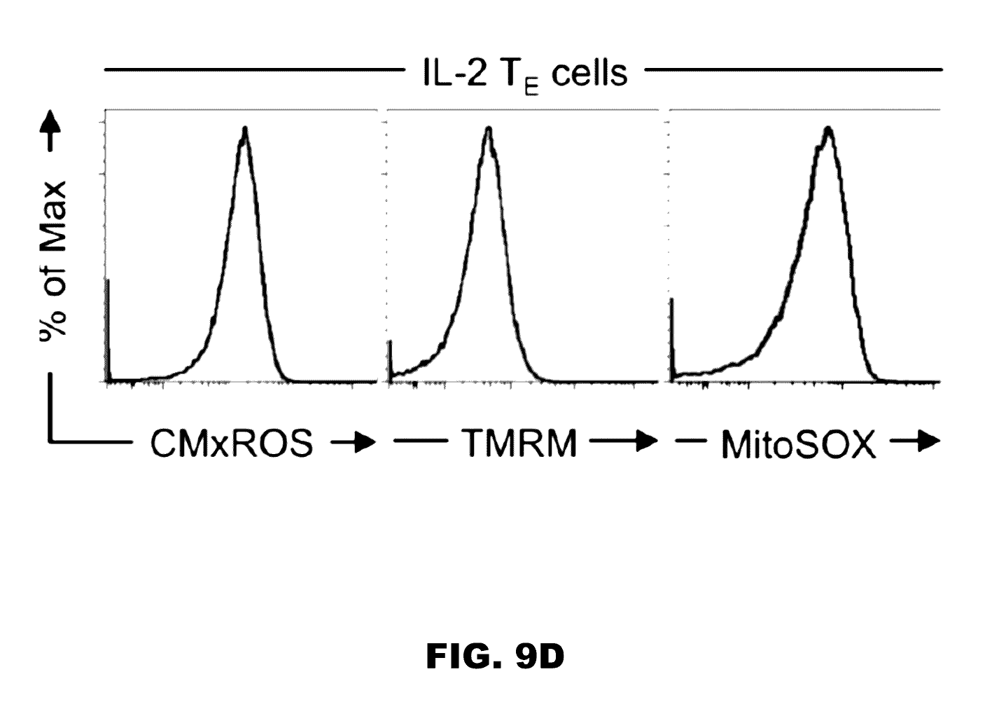

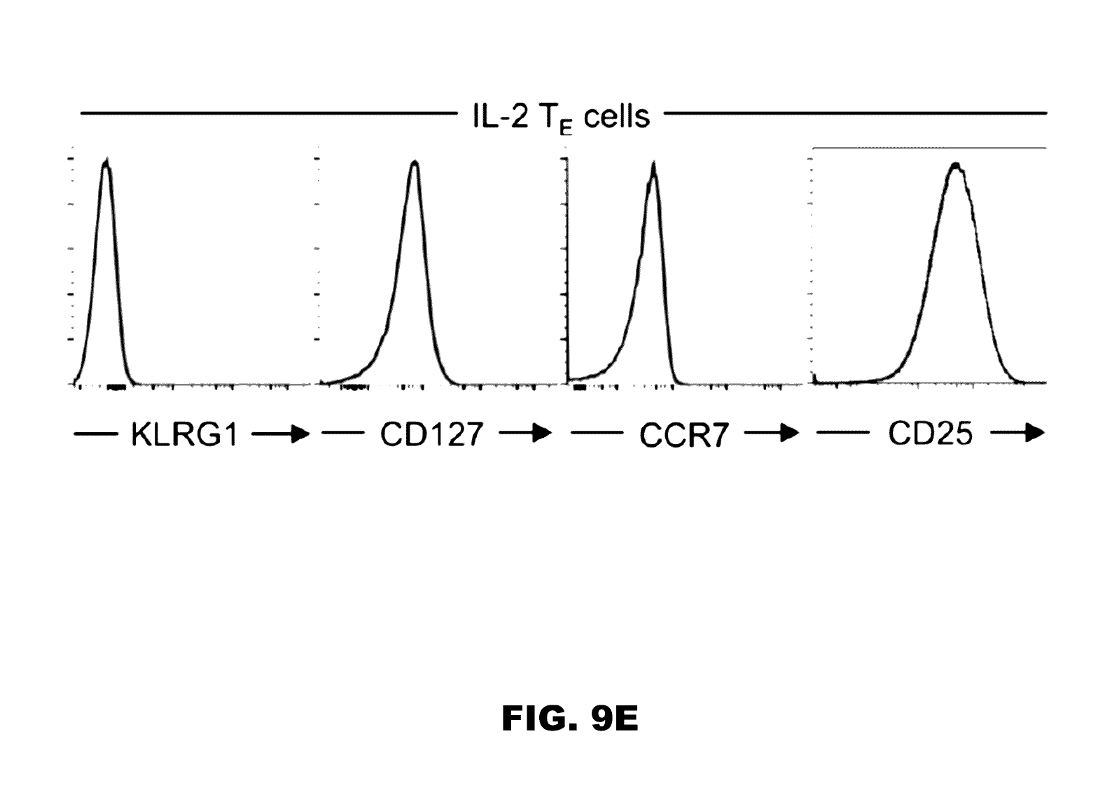

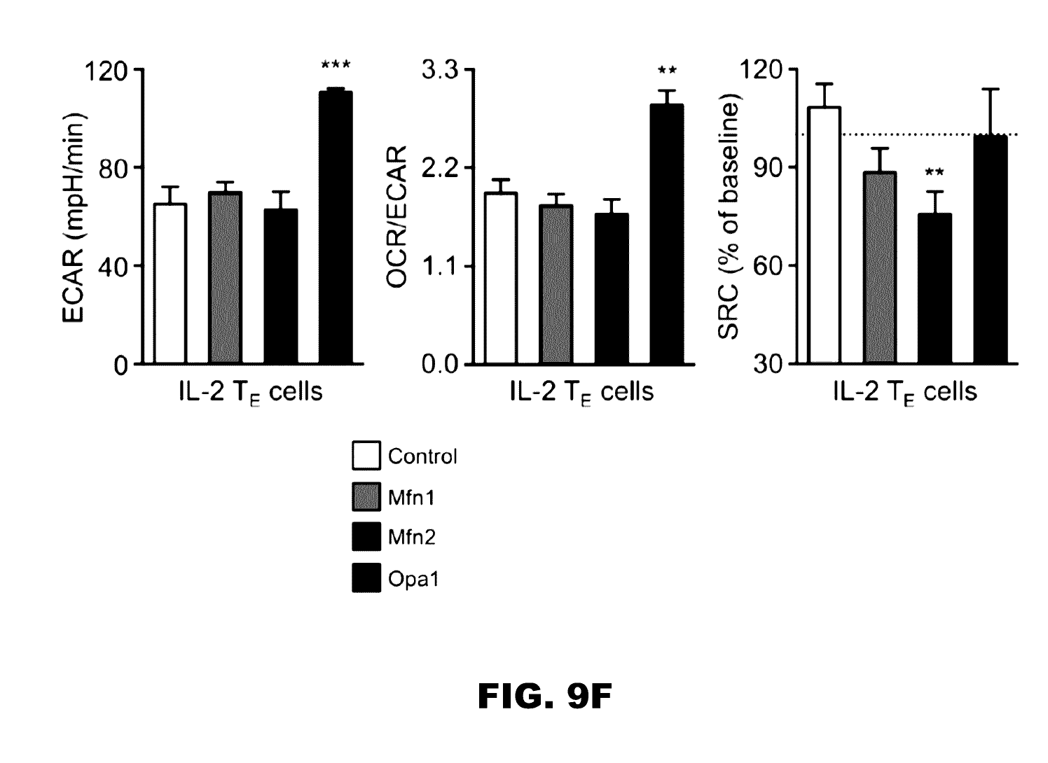

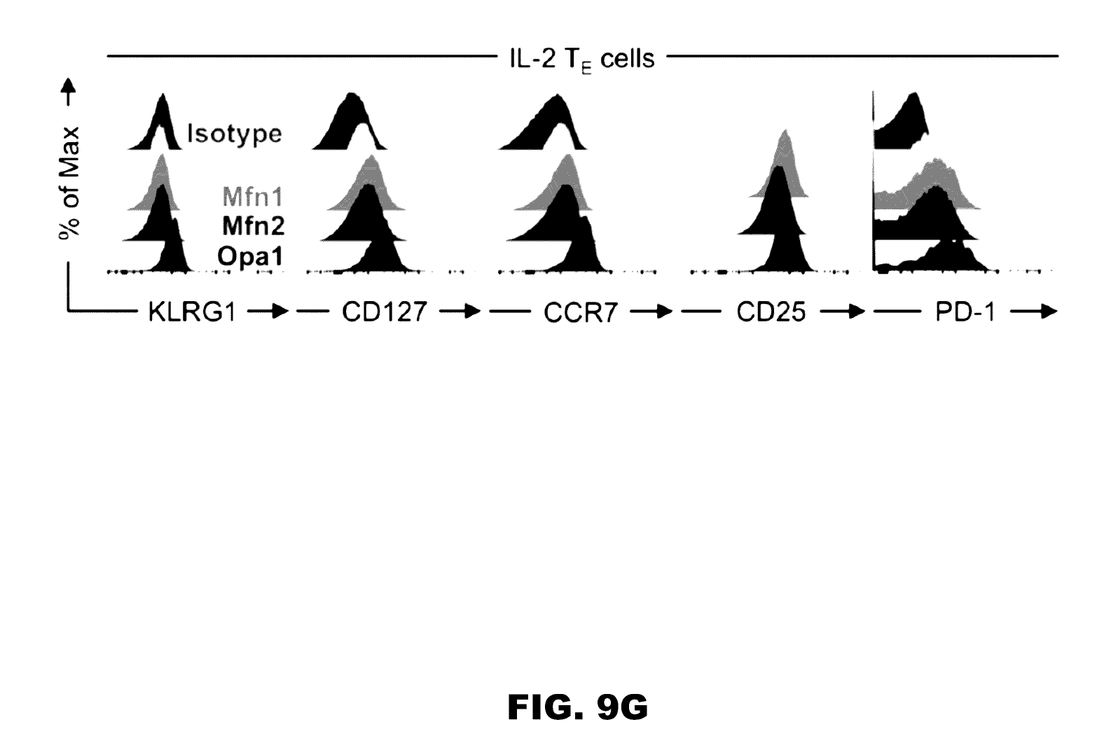

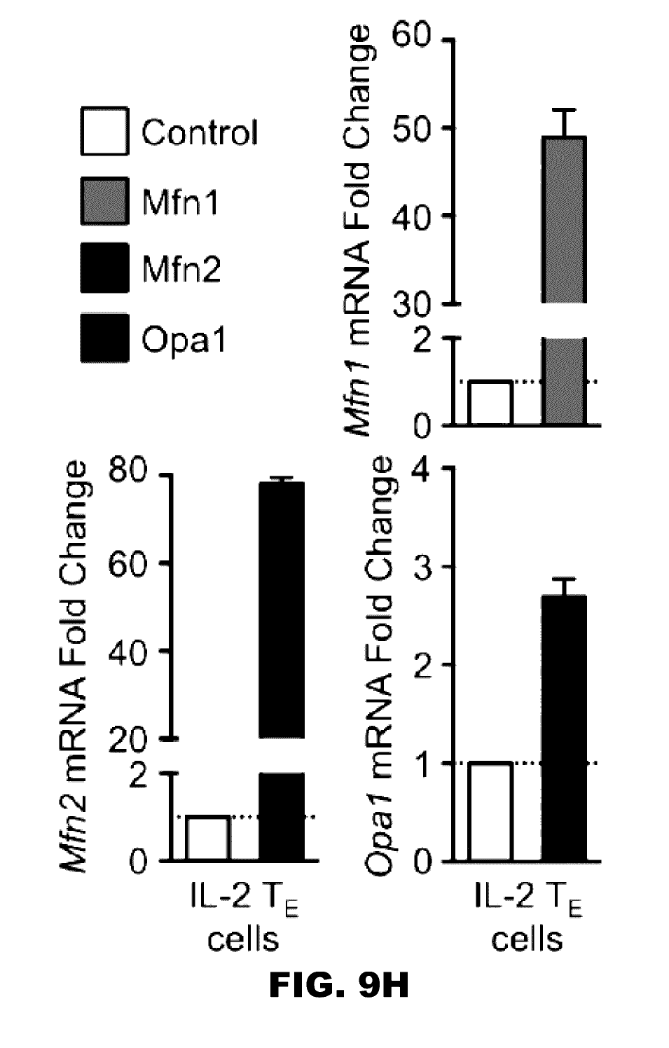

Genetic loss of function of Opa1 revealed that this protein is critical for T_{M} cell formation. Given the fused phenotype of mitochondria in these cells, we hypothesized that Opa1-mediated mitochondrial fusion supports the metabolism needed for T_{M} cell development. We used a gain of function approach to enhance mitochondrial fusion. Culturing T cells with the ‘fusion promoter’ M1, and the ‘fission inhibitor’ Mdivi-1 (FIG. 3A), induced mitochondrial fusion in IL-2 T_{E} cells, rendering them morphologically similar to IL-15 T_{M} cells (FIG. 3B). Treatment with these drugs enhanced other T_{M} cell properties in activated IL-2 T_{E} cells, including increased mitochondrial mass (FIG. 3C), OXPHOS and SRC (FIG. 3D), CD62L expression (FIG. 3E) and robust metabolic activity, as indicated by bioenergetic profiling of the cells in response to secondary stimulation with PMA/ionomycin, followed by addition of oligomycin (ATP synthase inhibitor), FCCP, and rotenone plus antimycin A (ETC complex I and III inhibitors), all drugs that stress the mitochondria (FIG. 3F and FIG. 9A) (Nicholls et al., 2010). However, we did not observe increased mtDNA in these cells (FIG. 9B). We found that ECAR and the OCR/ECAR ratio increased after drug treatment (FIG. 9C), indicating elevated metabolic activity overall, with a predominant increase in OXPHOS over glycolysis. While we observed these changes in mitochondrial activity, we did not measure any significant differences in mitochondrial membrane potential or ROS production after drug treatment (FIG. 9D). The expression of other activation markers were also not substantially affected, although a small decrease in KLRG1 and increase in CD25 was measured (FIG. 9E). Additionally, we performed a genetic gain of function experiment and transduced activated IL-2 T_{E} cells with retrovirus expressing Mfn1, Mfn2, or Opa1. Similar to enforcement of fusion pharmacologically, we found that cells transduced with Opa1 had more mitochondria (FIG. 3G) and OXPHOS (FIG. 3H), than empty vector control or Mfn-transduced T cells, as well as increased overall metabolic activity, with a predominant increase in OXPHOS over glycolysis (FIG. 9F). T_{M} cell associated markers such as CCR7 and CD127 were increased on transduced cells, as well as T_{E} cell proteins, such as PD-1 (FIG. 9G). We confirmed by mRNA expression that each target gene had increased expression after transduction over the control (FIG. 9G). Together our results show that mitochondrial fusion confers a T_{M} cell phenotype on activated T_{E} cells even in culture conditions that program T_{E} cell differentiation.

### Example 4. T Cell Mitochondrial Fusion Improves Adoptive Cellular Immunotherapy Against Tumors

A major consideration when designing adoptive cellular immunotherapy is to improve T cell fitness during ex vivo culture, so that when T cells are re-introduced into a patient they are able to function efficiently and persist for long periods of time (Restifo et al., 2012, Maus et al., 2014, O'Sullivan and Pearce, 2015). Our data showed that fusion-promoting drugs created metabolically fit T cells. We hypothesized that enforced fusion would also enhance the longevity of IL-2 T_{E} cells in vivo. To test this, we adoptively transferred control and M1+Mdivi-1 treated OT-I T cells into congenic mice and tracked donor cell survival. We found significantly more drug treated T cells in the spleen (FIG. 3I) and lymph nodes (FIG. 3J) 2 days after transfer. To determine if the persistence of these cells would be maintained better long term than control cells, we infected mice with LmOVA more than 3 weeks later and measured T cell responses against the bacteria. We found that drug-treated cells selectively expanded in response to infection (FIG. 3K) and could be recovered in significantly greater numbers in the spleen 6 days post-challenge (FIG. 3L).

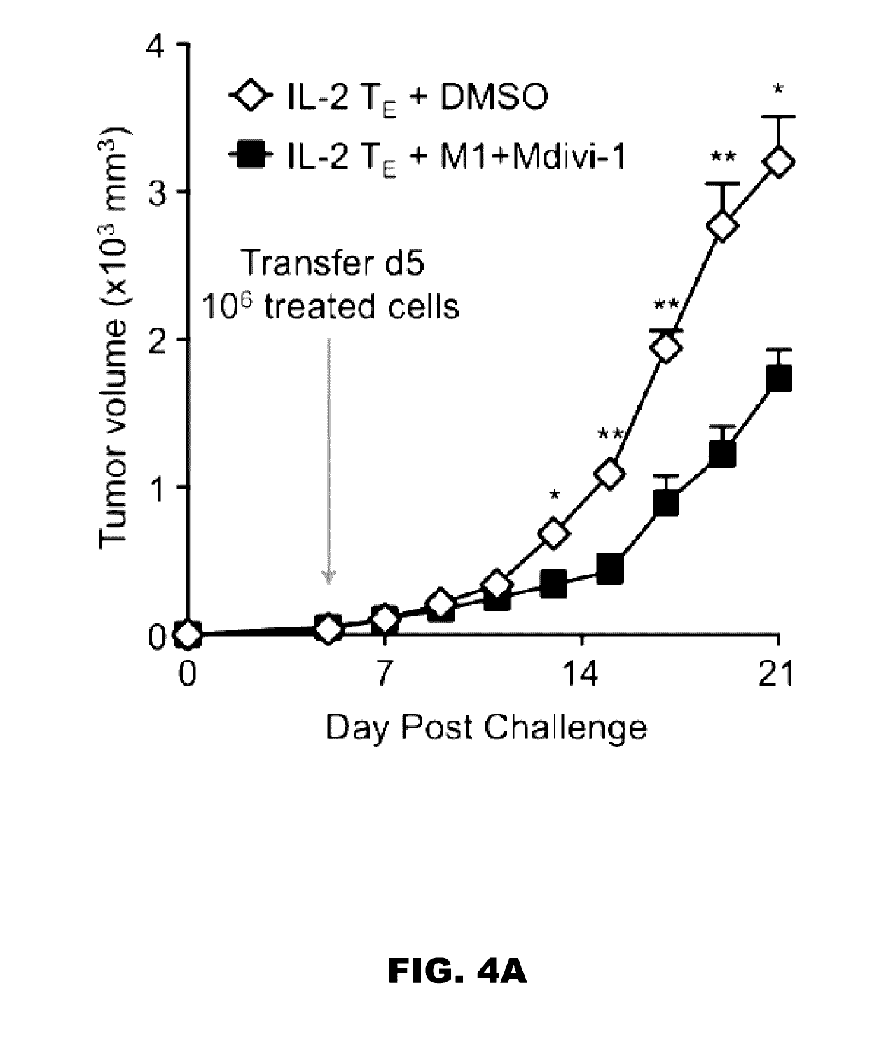

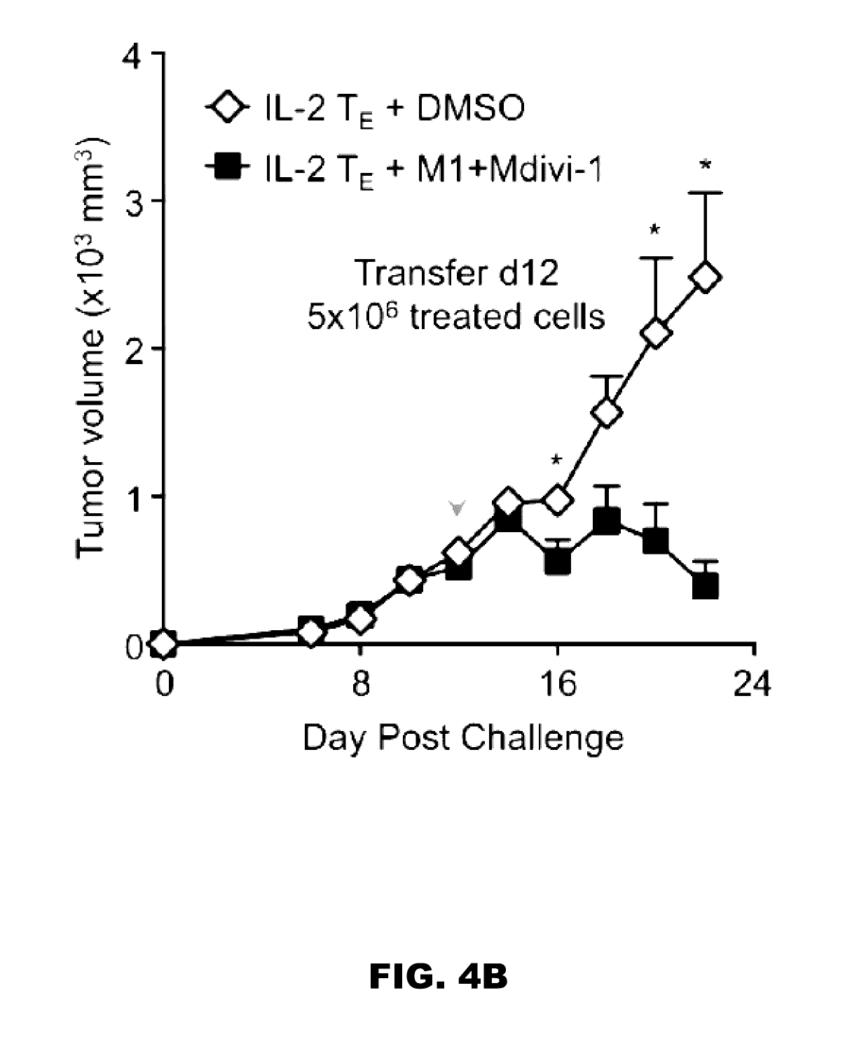

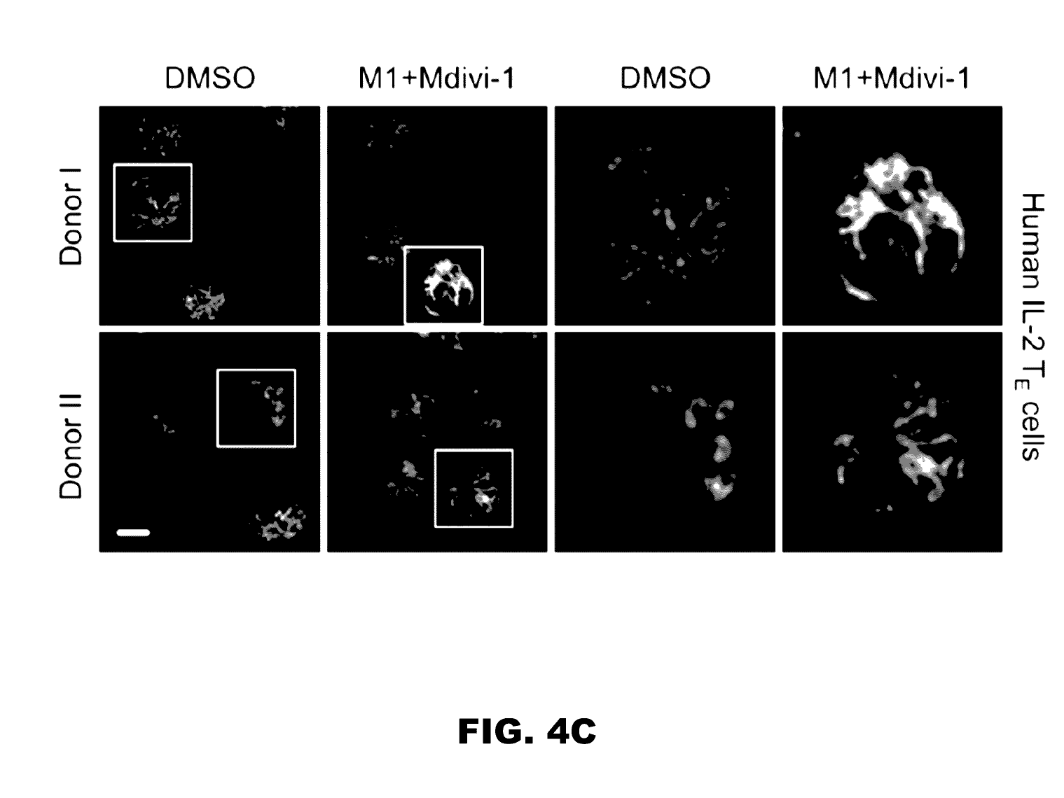

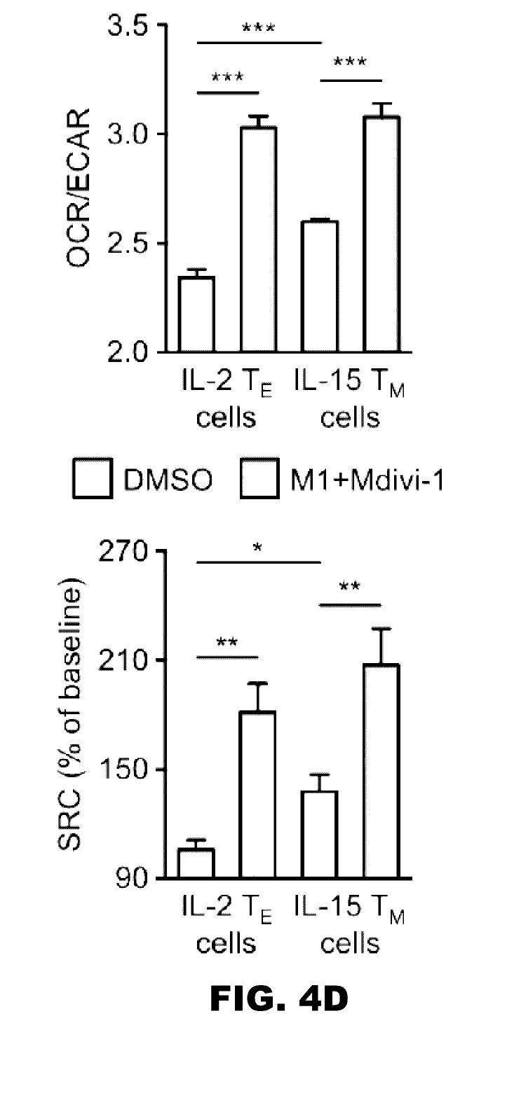

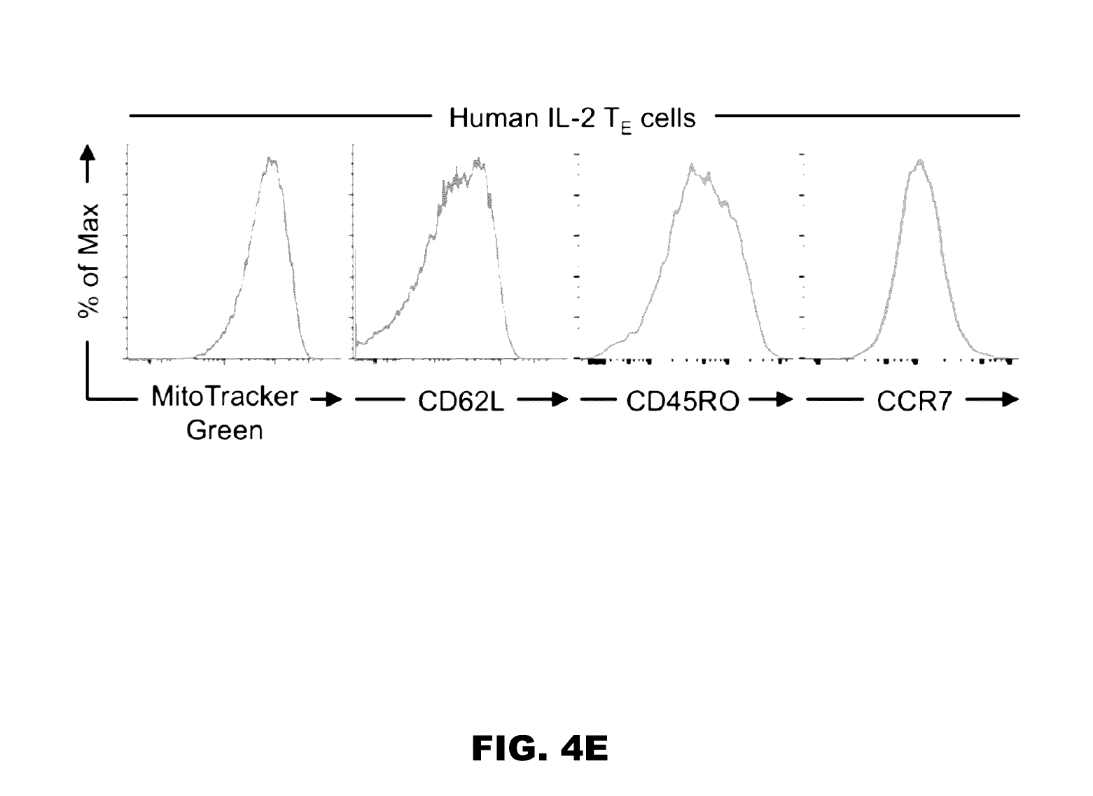

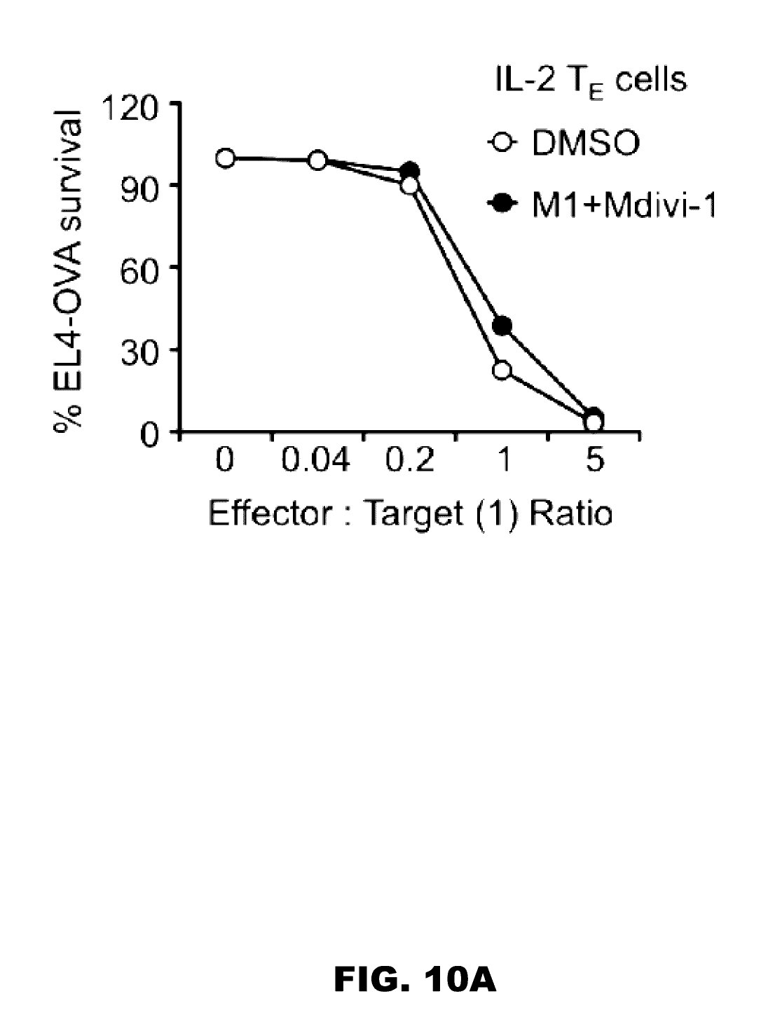

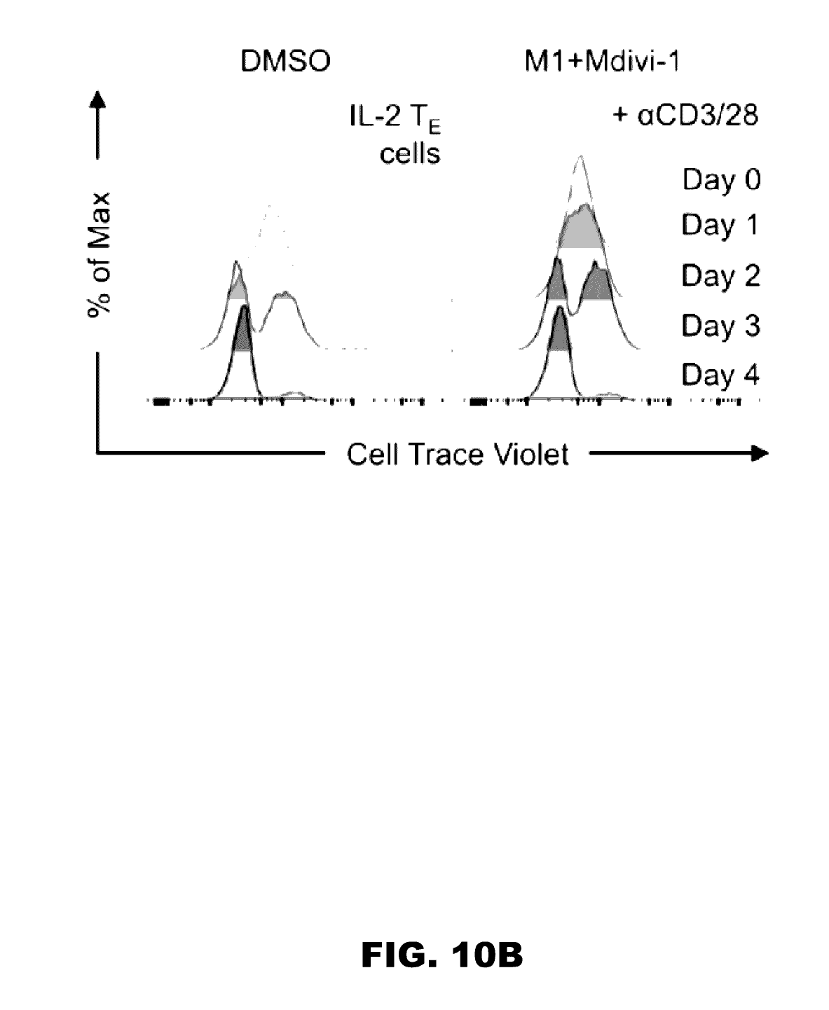

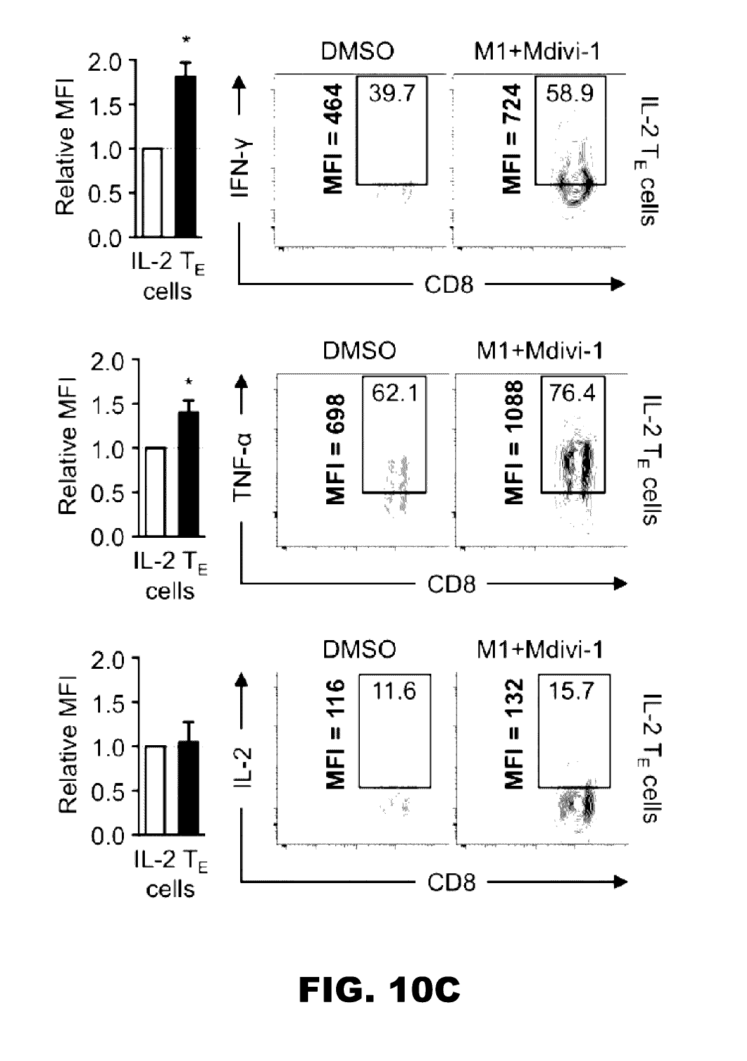

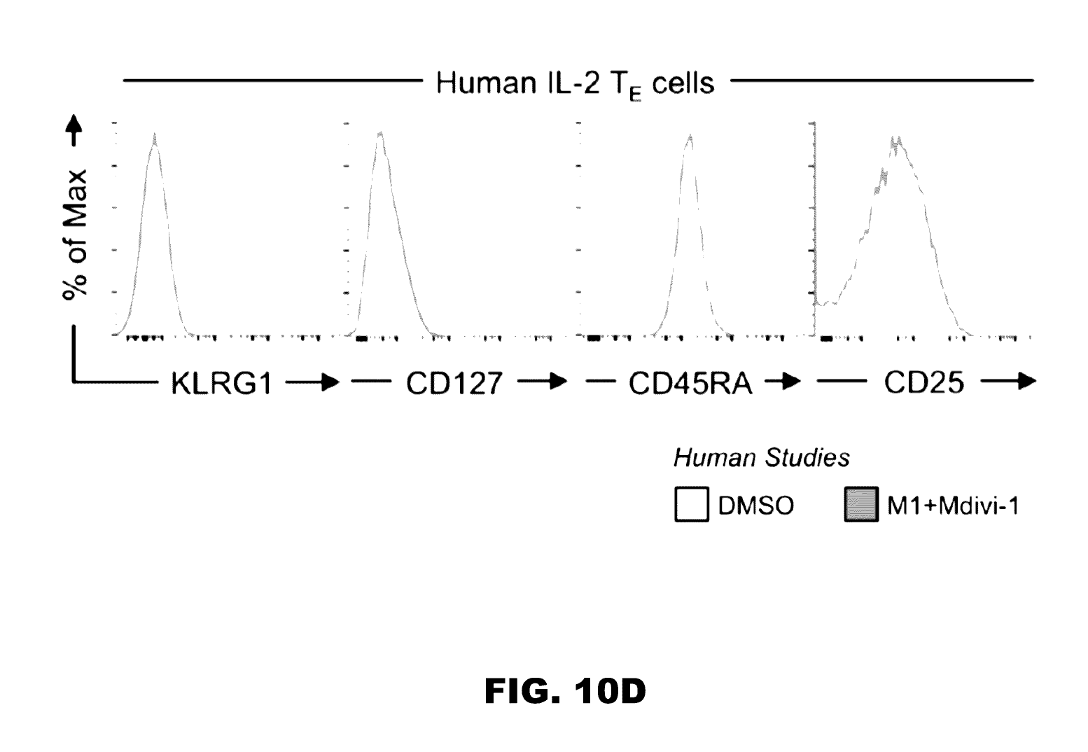

Next, we assessed whether these drugs could be used to promote T cell function in a model of adoptive cell immunotherapy. We injected EL4-OVA tumor cells into mice. Then either 5 or 12 days later we adoptively transferred IL-2 T_{E} cells that had been previously treated with DMSO or M1+Mdivi-1. In both settings, mice that had received ‘fusion-promoted’ T cells were able to control tumor growth significantly better than mice that had received control treated cells (FIG. 4A, FIG. 4B). The cytolytic ability (FIG. 10A) and proliferation (FIG. 10B) of the modified IL-2 T_{E} cells were similar to control cells, however, fusion enforced IL-2 T_{E} cells expressed significantly higher levels of IFN-γ and TNF-α when restimulated with PMA and ionomycin in vitro (FIG. 10C). We also exposed activated human T cells to M1+Mdivi-1 treatment in vitro and found that activated human IL-2 T_{E} cells had visibly more fused mitochondria (FIG. 4C), and exhibit the bioenergetic profile (FIG. 4D), and surface marker expression (FIG. 4E) characteristic of T_{M} cells, compared to control treated cells. Parameters such as mitochondrial mass (FIG. 4E) and other surface markers (FIG. 10D) were not significantly altered. These data suggest that promoting fusion in T cells may be translatable treatment for enhancing human therapy.

### Example 5. Mitochondrial Fusion Promotes T_{M} Cell Metabolism, but Opa1 is not Required for FAO

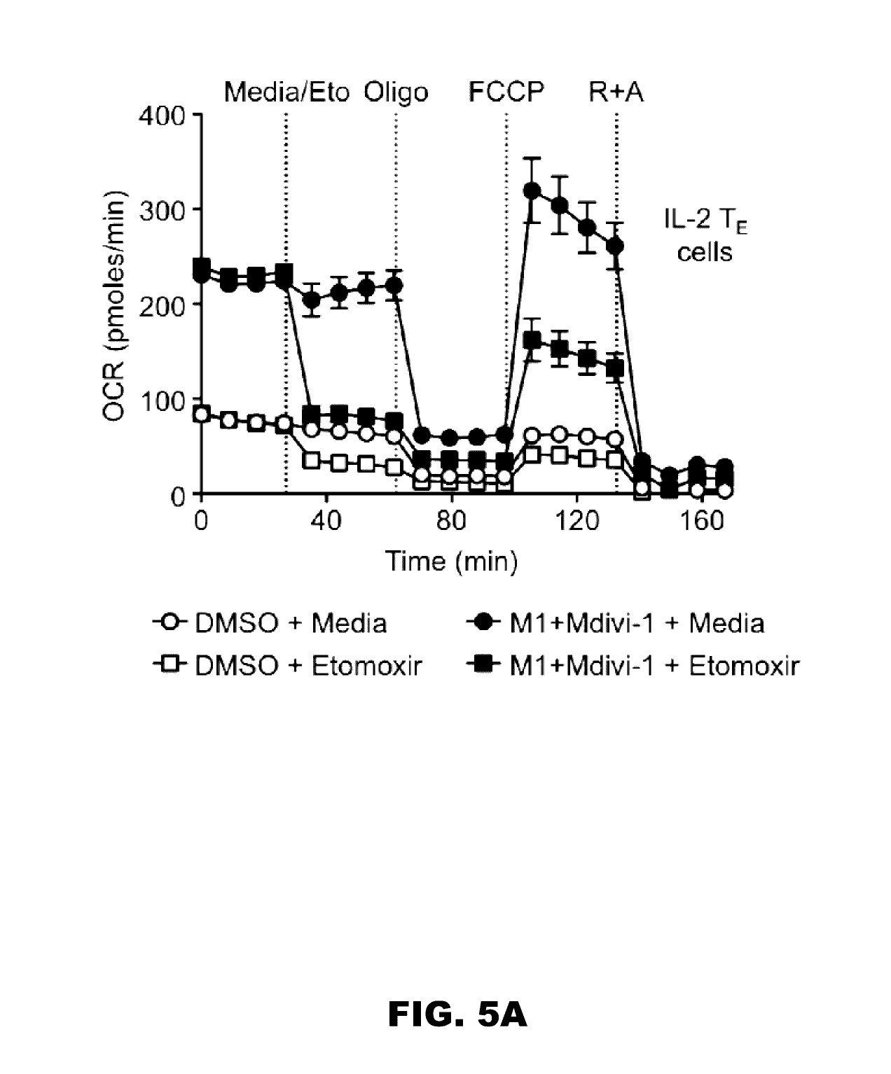

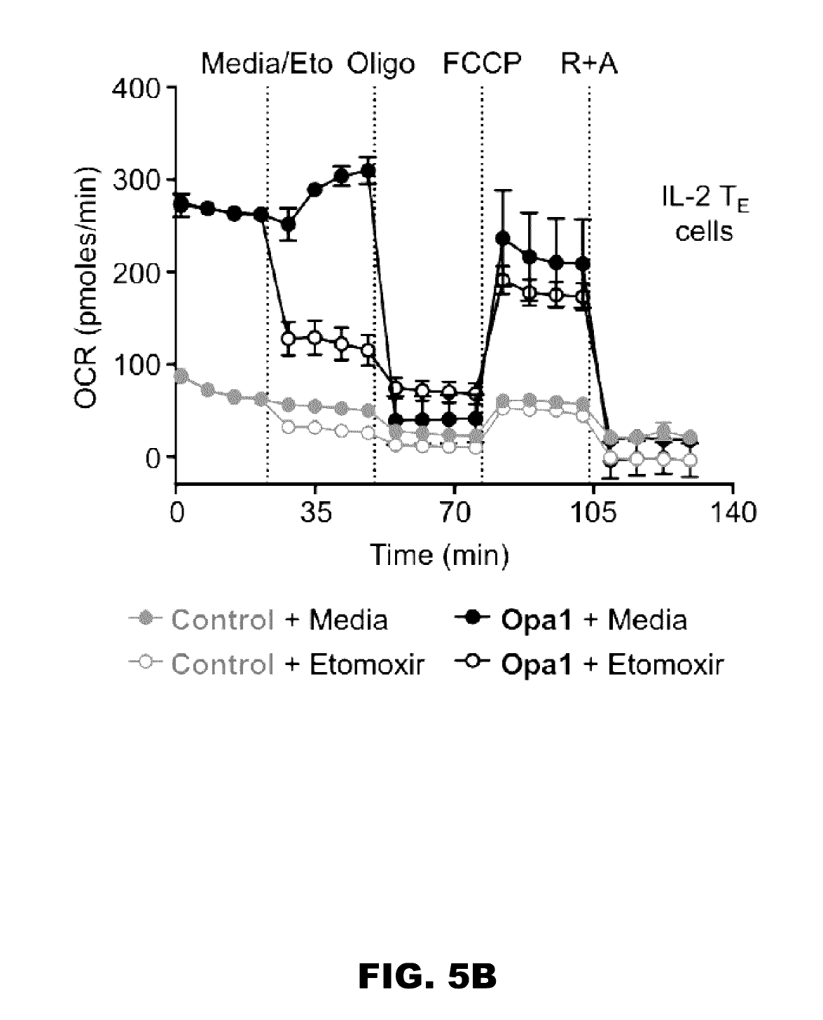

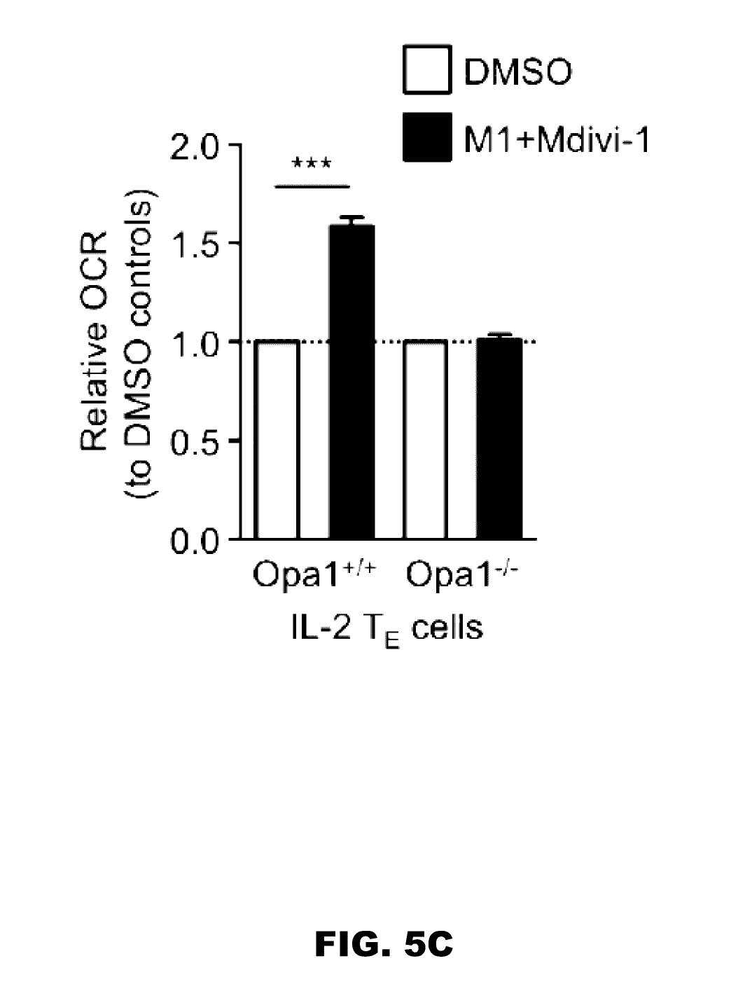

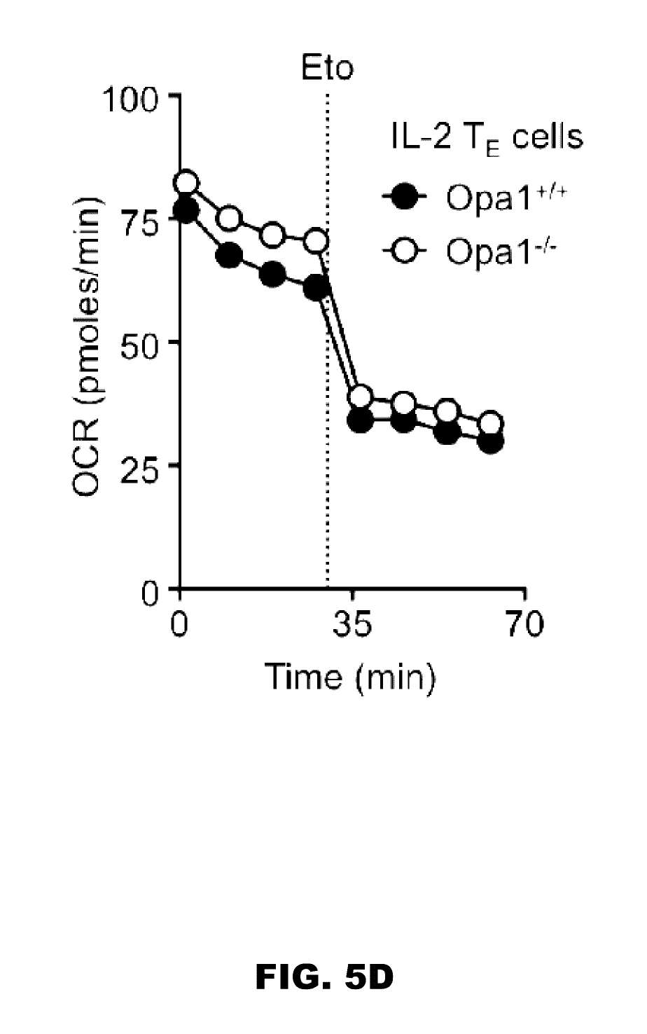

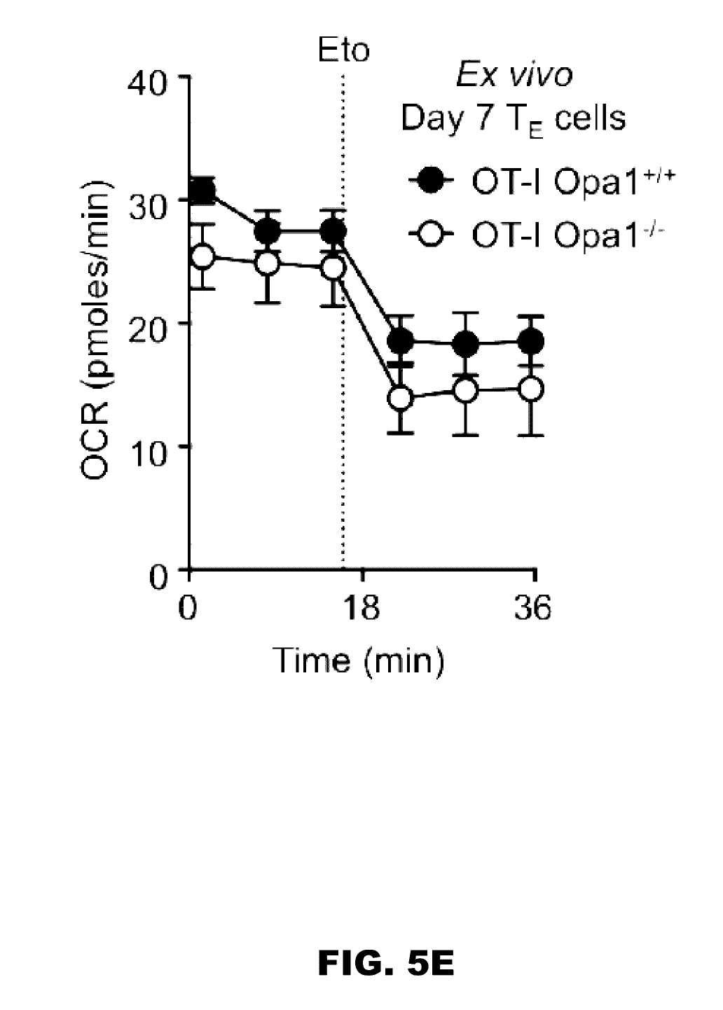

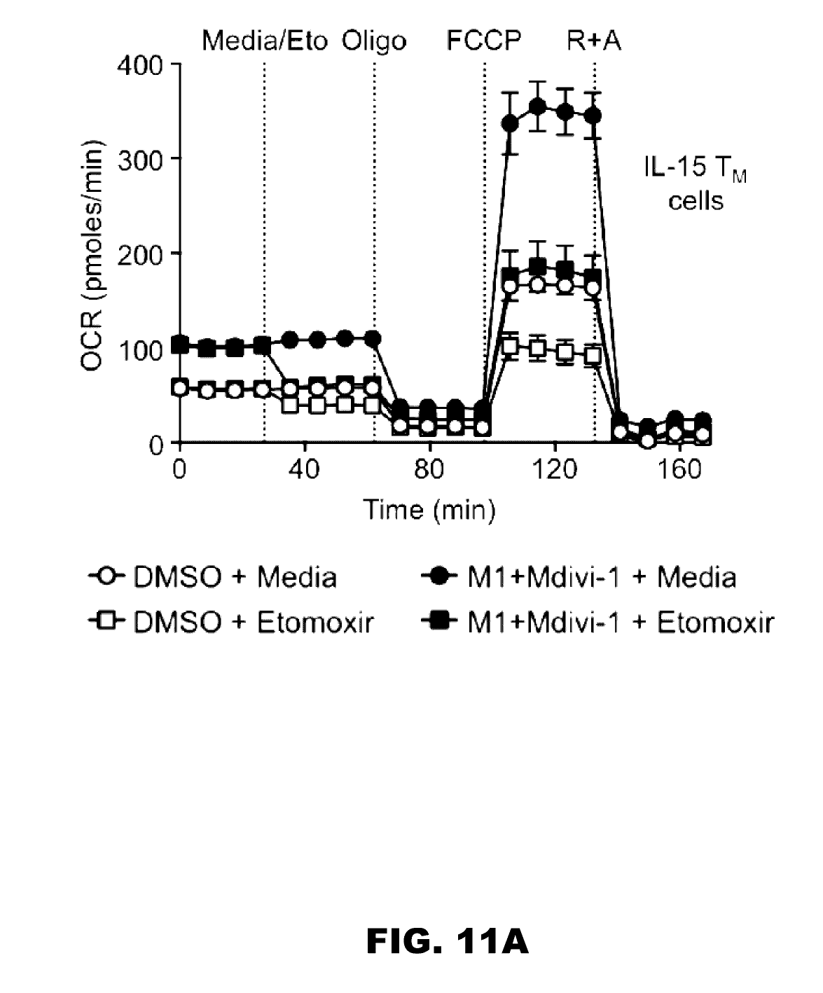

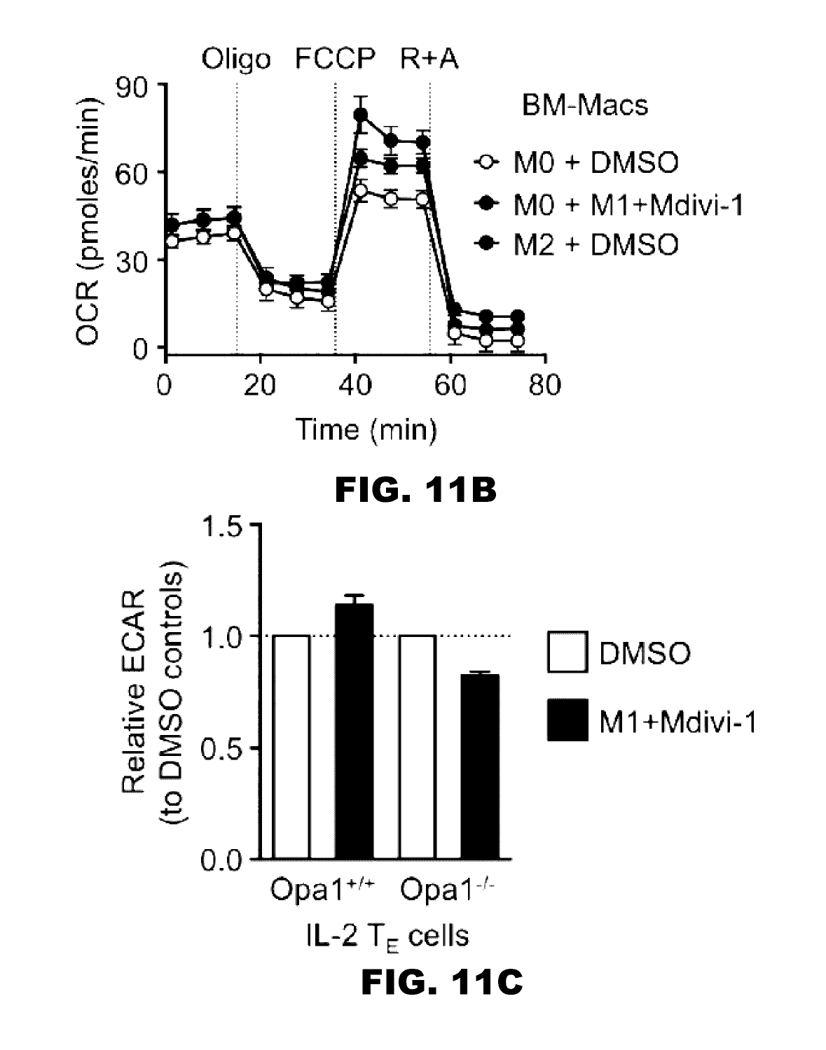

Our data showed that Opa1 was a necessary regulator of T_{M} cell development, but the question of precisely how Opa1 acted to support T_{M} cells remained. We hypothesized that mitochondrial fusion, via Opa1 function, was needed for FAO, as the engagement of this pathway is a requirement for T_{M} cell development and survival (Pearce et al., 2009, van der Windt et al., 2012, van der Windt et al., 2013). This hypothesis was not only based on our observations that these two processes seemed to be linked in T_{M} cells, but also knowledge that mitochondrial fusion is important for efficient FAO via lipid droplet trafficking under starvation conditions. We treated IL-2 T_{E} and IL-15 T_{M} cells with M1+Mdivi-1 or vehicle and then measured OCR in response to etomoxir, a specific inhibitor of mitochondrial long chain FAO (Deberardinis et al., 2006), and mitochondrial inhibitors. We found that the increased OCR and SRC evident in these cells after M1+Mdivi-1 treatment was due to augmented FAO (FIG. 5A and FIG. 11A). IL-2 T_{E} cells transduced with Opa1 also exhibited enhanced OCR that decreased in the presence of etomoxir compared to control cells (FIG. 5B). Bone marrow derived macrophages (BM-Macs) cultured with M1+Mdivi-1 also increased OCR and SRC to levels similar as M2 polarized macrophages, which engage FAO much like T_{M} cells do (FIG. 11B) (Huang et al., 2014). Importantly, M1+Mdivi-1 treatment did not increase OCR (FIG. 5C) and did not affect ECAR (FIG. 11C) in Opa1^{-/-} IL-2 T_{E} cells compared to controls, suggesting a requirement for Opa1 in augmenting OCR and FAO. However, in contrast to what we expected, when we assessed bioenergetics of Opa1^{+/+} and Opa1^{-/-} IL-2 T_{E} cells (FIG. 5D) and ex vivo isolated T_{E} cells (FIG. 5E), we found that both cell types are equally responsive to etomoxir. Our results show that while Opa1 could promote FAO in T cells, it was not compulsory for engagement of this metabolic pathway.

### Example 6. Mitochondrial Cristae Remodeling Signals Metabolic Adaptations in T_{M} Cells

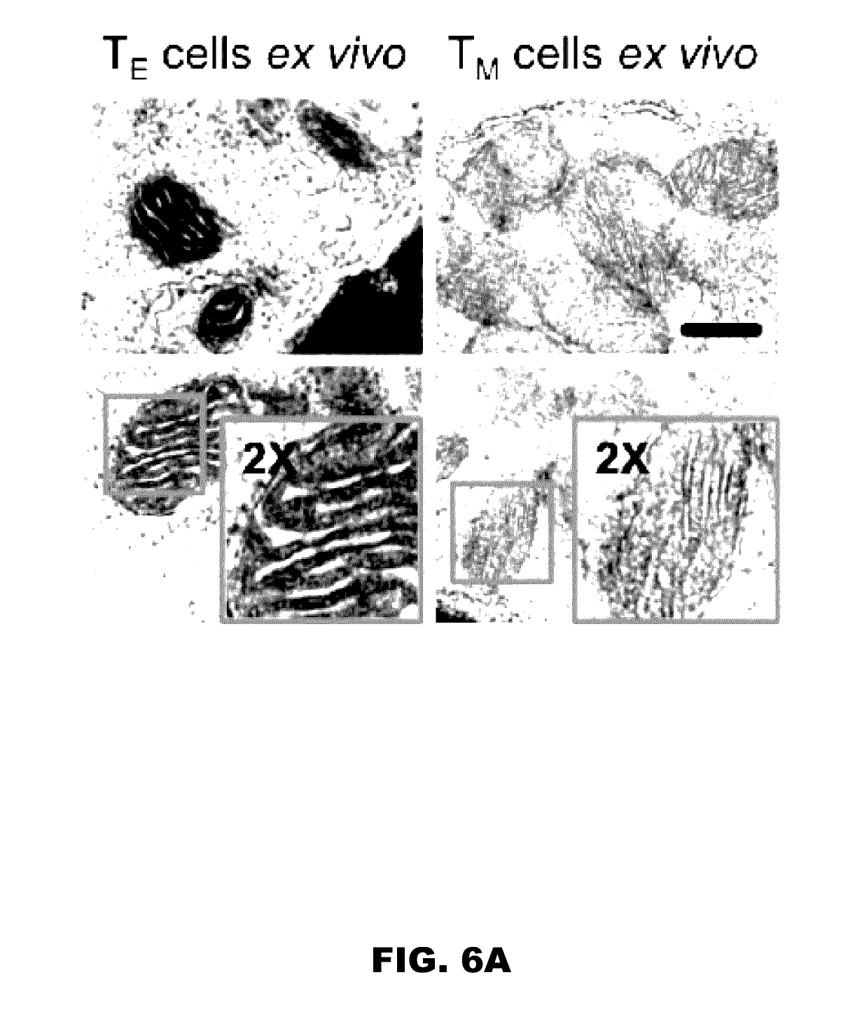

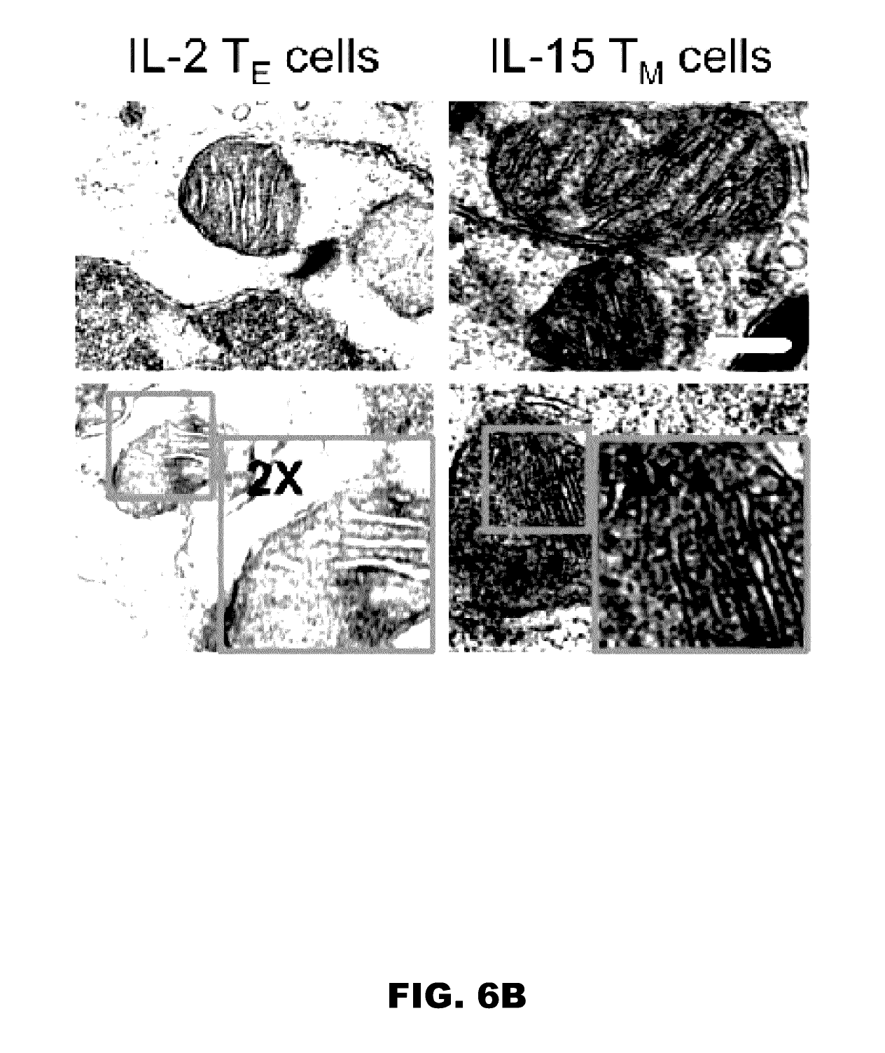

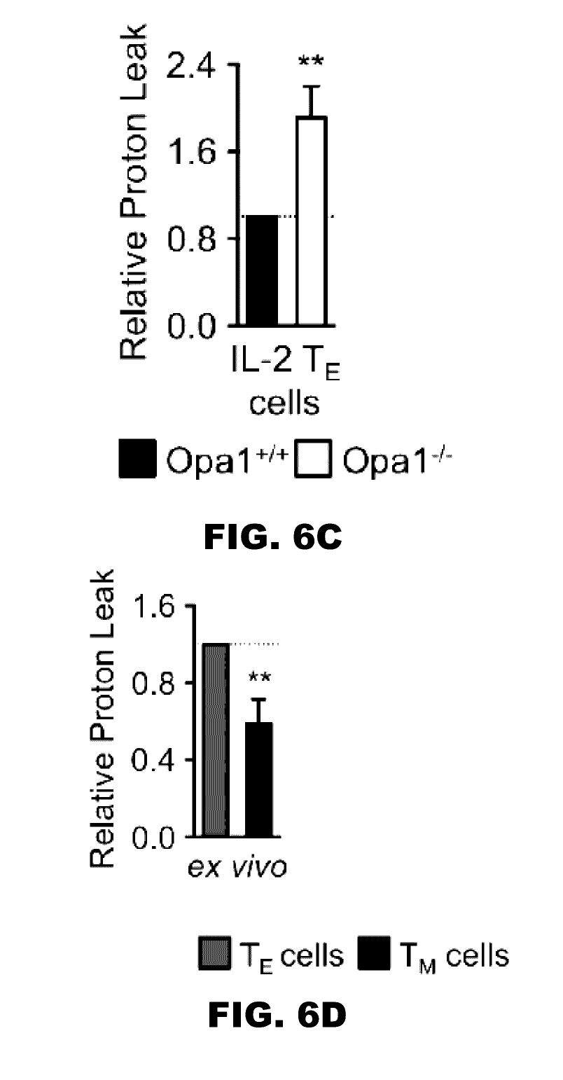

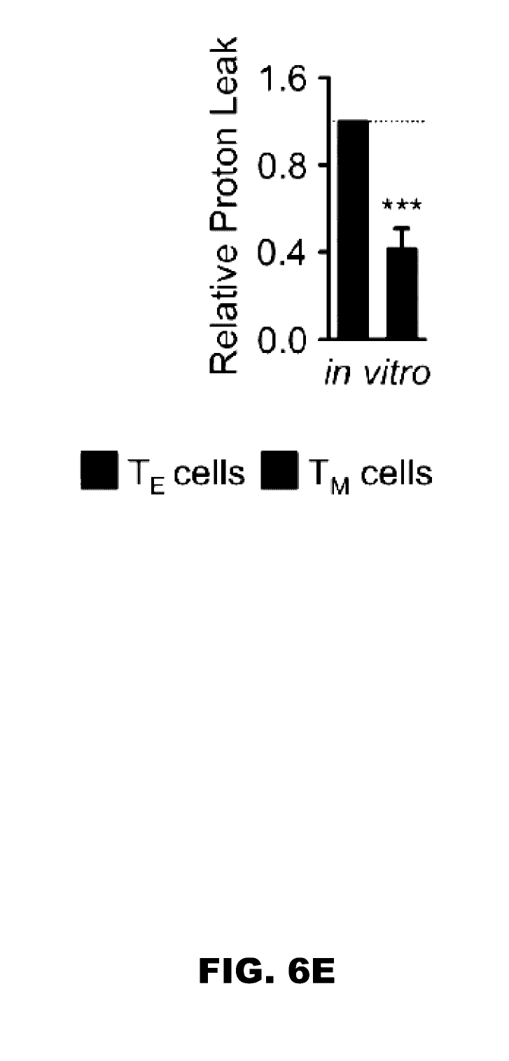

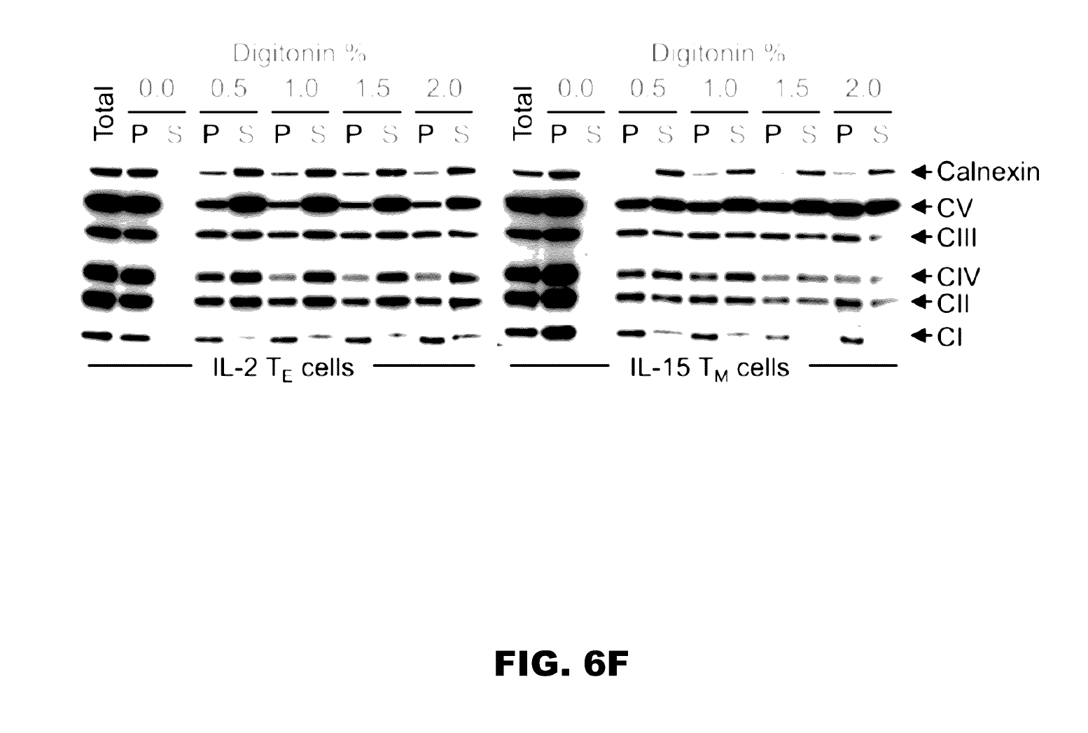

Opa1 is critical for inner mitochondrial membrane fusion, but also for other processes like cristae remodeling (Frezza et al., 2006, Cogliati et al., 2013, Varanita et al., 2015). We observed major changes in cristae morphology in the Opa1^{-/-} T cells (FIG. 2B). Given the importance of Opa1 function in T_{M} cell development (FIG. 2), we further assessed cristae morphology in T_{E} and T_{M} cells isolated ex vivo after LmOVA infection (FIG. 6A), as well as in IL-2 T_{E} and IL-15 T_{M} cells (FIG. 6B), and found that T_{E} cells had many cristae with what appeared to be slightly wider, or more loosely organized intermembrane space, than T_{M} cells. It has been found that Opa1 overexpression induces cristae tightening and close association of ETC complexes in the inner mitochondrial membrane (Cogliati et al., 2013, Civiletto et al., 2015). Therefore, we surmised that in the absence of Opa1, cristae disorganization leads to dissociation of ETC complexes, and subsequently less efficient ETC activity, in T cells (FIG. 2C). We assessed OCR after oligomycin in relation to OCR after rotenone plus antimycin A treatment (i.e. proton leak), which indicates the coupling efficiency of OXPHOS with mitochondrial ATP production. Consistent with decreased OXPHOS efficiency, we observed increased proton leak in Opa1^{-/-} T cells compared to control cells (FIG. 6C). This was also true for ex vivo isolated T_{E} cells when compared to T_{M} cells (FIG. 6D), as well as IL-2 T_{E} and IL-15 T_{M} cells (FIG. 6E). Together these data suggest that there are cristae differences between T_{E} and T_{M} cells which may contribute to their distinct metabolic phenotypes.

### Example 7. T_{M} Cells Maintain Tight Cristae with Closely Associated ETC Complexes

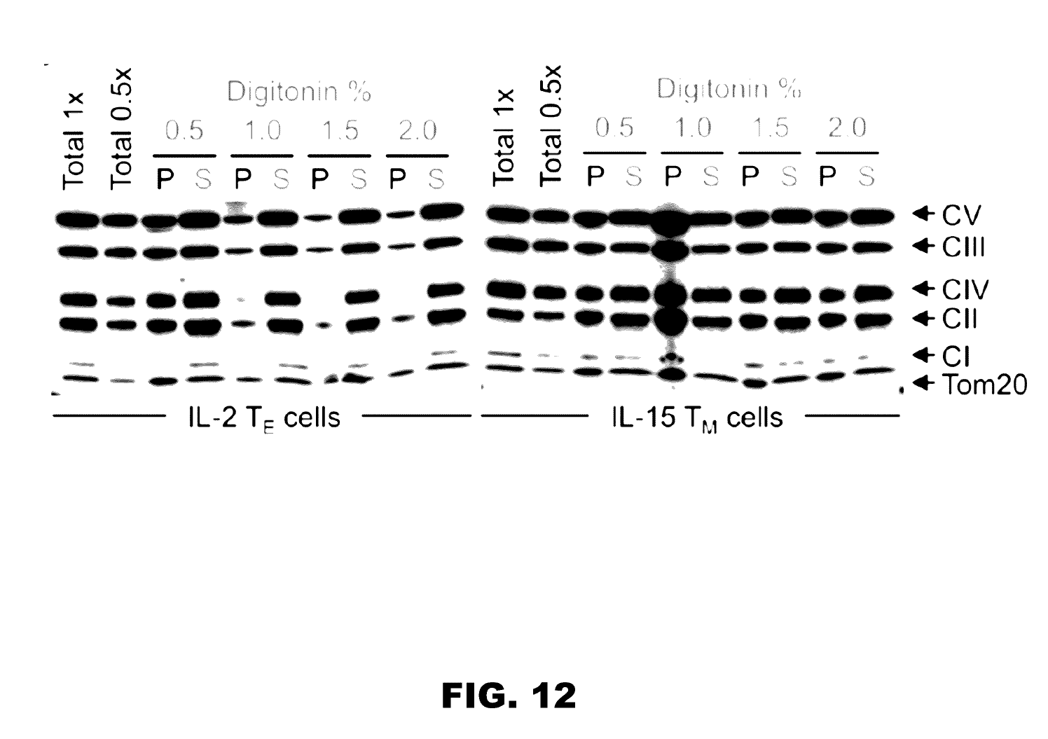

Our data suggested that unlike T_{E} cells, T_{M} cells have tight cristae with close association of the ETC complexes. To investigate this biochemically, we treated native lysates of IL-2 T_{E} and IL-15 T_{M} cells with increasing concentrations of digitonin to disrupt all cellular membranes (including mitochondrial). The crude membrane-bound fraction was separated from solubilized proteins by centrifugation. Both pellet and soluble supernatant fractions were loaded on a denaturing reducing gel and then probed for various mitochondrial proteins by western blot. We found that mitochondria in IL-2 T_{E} cells were susceptible to digitonin disruption, indicated by the fact that ETC complex proteins became less detectable in the pellet, and amplified in the soluble fraction, in 0.5% detergent (FIG. 6F, FIG. 12). This was in contrast to IL-15 T_{M} cells, where ETC proteins did not solubilize to the same extent as those in IL-2 T_{E} cells into the supernatant, even when 2% digitonin was used. To investigate whether this phenomenon was unique to the mitochondrial compartment, we also probed for the ER integral protein calnexin and found that it solubilized similarly in 0.5% digitonin in both cell types. Overall these data suggest that there is more exposed mitochondrial membrane between proteins in IL-2 T_{E} cells than in IL-15 T_{M} cells, and correlate with the idea that T_{M} cells have tight cristae, which yields efficient ETC activity supporting its distinct metabolic phenotypes.

### Discussion for the Examples

Although T_{M} cells rely on FAO for development and survival, precisely why T_{M} cells utilize FAO and the signals that drive the induction of aerobic glycolysis in T_{E} cells remain unclear. Our data suggest that manipulating the structure of a single organelle can have profound consequences that impact metabolic pathway engagement and ultimately, the differentiation of a cell. We found that Opa1 regulated tight cristae organization in T_{M} cells, which facilitated efficient ETC activity. We originally hypothesized that Opa1 would be an obligate requirement for FAO. However, we found that Opa-1^{-/-} IL-2 T_{E} cells and ex vivo T_{E} cells generated during infection utilized FAO to the same level as cells expressing Opa1. While this was true for T_{E} cells, this may not be the case for T_{M} cells, whose survival is severely impaired in vitro and in vivo when deficient in Opa1. It is possible that Opa1^{-/-} T cells are unable to form T_{M} cells because they cannot efficiently engage FAO under the metabolic constraints imposed during T_{M} cell development. Previous studies point to the existence of a ‘futile’ cycle of fatty acid synthesis (FAS) and FAO within T_{M} cells (O'Sullivan et al., 2014, Cui et al., 2015) whereby carbon derived from glucose oxidation is used to build fat that is subsequently burned by mitochondria for fuel. T_{M} cells have a lower overall metabolic rate than T_{E} cells, and tightly configured cristae might be important to ensure that any pyruvate generated will efficiently feed into the TCA cycle not only for reducing equivalents, but also for deriving citrate for FAS. Without tight cristae and efficient ETC activity, electrons may loiter in the complexes, causing more ROS, which could be damaging (Chouchani et al., 2014).

We did not observe a defect in T_{M} cell survival in Mfn1 and Mfn2 deficient T cells, but this does not exclude the possibility that OMM fusion or additional activities ascribed to each of these proteins are not important. Mfn1 and Mfn2 form both homotypic and heterotypic interactions, suggesting that in the absence of one protein, the other can compensate (Chen et al., 2003). Our preliminary data assessing Mfn1/2 expression in Mfn1^{-/-} and Mfn2^{-/-} T cells indicate that this might be occurring (data not shown). However, our results clearly show that unlike Opa1^{-/-} T cells, in vitro cultured Mfn1^{-/-} or Mfn2^{-/-} T cells do not have a survival defect when differentiated in IL-15 (FIG. 2A), even though, like Opa1^{-/-} T cells, they are more glycolytic and OXPHOS-impaired compared to controls (data not shown). Further investigation using Mfn1/2 double knockouts is underway in our laboratory to examine whether a lack of both proteins, and presumably total OMM fusion, impairs T_{M} cell development in similar fashion as deficiency in inner membrane fusion. Our imaging data showed that T_{M} cells maintained extended fused mitochondrial networks, suggesting that OMM fusion also has a compulsory role in T_{M} cell development. However, unlike Opa1, retroviral expression of Mfn1 and Mfn2 did not confer a T_{M} cell phenotype in T_{E} cells.

The question of what signals drive T cell structural remodeling of mitochondria in the first place still remains. In the case of T_{M} cell development, initial withdrawal of activating signals and growth factors may induce fusion, consistent with previous reports that starvation induces mitochondrial hyperfusion (Rambold et al., 2011 b, Rambold et al., 2015), an effect we also observe in T_{E} cells after IL-2 withdrawal (data not shown). However, pro-survival signals from cytokines such as IL-15 or IL-7 are needed to sustain T_{M} cell viability and metabolically remodel these cells for FAS and FAO via increased CPT1a (van der Windt et al., 2012) and aquaporin 9 expression (Cui et al., 2015). Factors such as these may enforce the fused state and would be consistent with our observations that activated T cells subsequently cultured in IL-15 become more fused over time (FIG. 1D). Another possibility is that Opa1 is activated via sirtuin 3 (SIRT3) under metabolically stressful conditions (Samant et al., 2014). Sirtuins are post-translational modifiers that are activated by NAD⁺, directly tying their activity to the metabolic state of the cell (Houtkooper et al., 2012, Wang and Green, 2012). Our previous work demonstrated that the available NAD⁺ pool is higher in T_{M} cells (van der Windt et al., 2012), which could correlate with this scenario.

We show that IL-2 T_{E} cells have a mitochondrial structure that is more susceptible to digitonin disruption when compared to IL-15 T_{M} cells, which suggests a more exposed membrane with less densely packed protein complexes. This relatively enhanced permeability however, does not mean that their mitochondria are damaged, or unable to function. In fact, although T_{E} cells have less efficient OXPHOS in terms of how it is coupled to ATP synthesis, T_{E} cells are very metabolically active with high OCR and ECAR (Chang et al., 2013, Sena et al., 2013). Our experiments involving pharmacological enforcement of mitochondrial fusion promoted OCR and SRC (and ECAR, albeit to a lesser extent) in IL-2 T_{E} cells. The drug modified cells maintained full T_{E} cell function with no effect on their cytolytic ability or proliferation, but possessed enhanced cytokine expression. Fusion and/or cristae tightening boosted the T_{E} cells' oxidative capacity, endowing them with longevity and persistence, while their higher aerobic glycolysis supported increased cytokine production, which may explain their superior antitumor function.

Our data suggest a model where morphological changes in mitochondria are a primary signal that shapes metabolic reprogramming during cellular quiescence. When cristae are tightly configured, the ETC works efficiently and maintains entrance of pyruvate into the mitochondria with a favorable redox balance. In this case, cristae morphology as a result of fusion directs T_{M} cell formation and retains these cells in a quiescent state. Thus, mitochondrial dynamics control the balance between metabolic pathway engagement and T cell fate.

### Methods for the Examples

Mice and Immunizations:

C57BL/6, C57BL/6 CD45.1, C57BL/6 CD90.1, photo-activatable mitochondria (PhAM), and major histocompatibility complex (MHC) class I-restricted OVA specific TCR OT-I transgenic mice were purchased from The Jackson Laboratory. Mfn1 and Mfn2 conditional floxed mice were obtained from Dr. David C. Chan (California Institute of Technology, Pasadena, Calif.). Opa1 conditional floxed mice were obtained from Dr. Hiromi Sesaki (Johns Hopkins University School of Medicine, Baltimore, Md.). All conditional floxed mice were crossed to OT-I CD4 Cre transgenic mice to generate OT-I Mfn1^{F/F} CD4 Cre, OT-I Mfn2^{F/F} CD4 Cre, and OT-I Opa1^{F/F} CD4 Cre mice. All mice were bred and maintained under specific pathogen free conditions under protocols approved by the AAALAC accredited Animal Studies Committee of Washington University School of Medicine, St. Louis, Mo. USA and the Animal Welfare Committee of the Max Planck Institute of Immunobiology and Epigenetics Freiburg, Germany. Age matched mice were injected intraperitoneally (i.p.) or intravenously (i.v.) as indicated with a sublethal dose of 1×10⁶ colony forming units (CFU) of recombinant Listeria monocytogenes expressing OVA deleted for actA (LmOVA) for primary immunizations and challenged with 5×10⁷ CFU for secondary immunizations. For tumor experiments, 1×10⁶ EL4 lymphoma cells expressing OVA (EL4-OVA) were injected subcutaneously (s.c.) into the right flank of mice.

Cell Culture and Drug Treatments:

OT-I splenocytes were activated with OVA-peptide (SIINFEKL (SEQ ID NO:1), New England Peptide) and IL-2 (100 U/mL) for 3 days and subsequently cultured in the presence of either IL-2 or IL-15 (10 ng/mL) for an additional 3 days in TCM (RPMI 1640 media supplemented with 10% FCS, 2 mM L-glutamine, 100 U/mL penicillin/streptomycin, and 55 μM β-mercaptoethanol). For drug treatment experiments, vehicle control (DMSO) or 10 μM Mdivi-1+20 μM M1 (Sigma) were added to cultures daily starting on day 3. For in vitro survival assays, cells were activated for 3 days as described, then cultured in either IL-2 at 5×10⁴ cells/mL or IL-15 at 1×10⁵ cells/mL in 96 well round bottom plates. Survival was analyzed by 7AAD exclusion using flow cytometry. Bone marrow cells were differentiated for 7 days into BM-Macs by culturing in complete medium (RPMI 1640 supplemented with 10% FBS, 100 U/mL penicillin/streptomycin, 2 mM L-glutamine) with 20 ng/mL mouse macrophage colony-stimulating factor (M-CSF; PeproTech). BM-Macs were stimulated with 20 ng/mL IL-4 (PeproTech).

Flow Cytometry and Spinning Disk Confocal Microscopy:

Fluorochrome-conjugated monoclonal antibodies were purchased from eBioscience, BD Pharmingen, or Biolegend and staining performed as previously described (Chang et al., 2015). OVA-specific CD8⁺ T cells from spleen, lymph node, or blood were quantified by direct staining with H2-K^{b}OVA257-264 (K^{b}OVA) MHC-peptide tetramers. MitoTracker, TMRE, CMxROS, MitoSOX, and Hoechst staining was performed according to the manufacturer's instructions (Life Technologies). Nos2 protein levels in BM-Macs were quantified after fixation and permeabilization using the transcription factor staining buffer set (eBioscience) and a directly conjugated antibody against Nos2 (clone CXNFT, eBioscience). Cells were collected on FACS Calibur, Canto II, LSR II, and Fortressa flow cytometers (BD Biosciences) and analyzed using FlowJo (TreeStar) software. Cells were sorted using a FACS Aria II. Cells were imaged live on glass bottom dishes coated with fibronectin or poly-D-lysine (Sigma) in TCM containing IL-2 or IL-15 (MatTek) using a LSM 510 META confocal scanning microscope (Zeiss), an Olympus Confocal Microscope FV1000, or a Zeiss spinning disk confocal with a Evolve (EMCCD) camera. Cells were kept in a humidified incubation chamber at 37° C. with 5% CO₂ during image collection. Images were deconvolved and analyzed using ImageJ (NIH). Brightness and contrast were adjusted in Adobe Photoshop CS.

Transmission Electron Microscopy:

Cells were fixed in 2% paraformaldehyde, 2.5% glutaraldehyde in 100 mM sodium cocodylate containing 0.05% malachite green. Following fixation, samples were washed in cocodylate buffer and post fixed in 1% osmium tetroxide. After extensive washing in H₂O, samples were stained with 1% aqueous uranyl acetate for 1 hour and washed again. Samples were dehydrated in ethanol and embedded in Eponate 12 resin (Ted Pella). Cut sections were stained with uranyl acetate and lead citrate and then imaged using a JOEL 1200 EX transmission electron microscope equipped with an 8 MP ATMP digital camera (Advanced Microscopy Techniques).

Metabolism Assays:

Oxygen consumption rates (OCR) and extracellular acidification rates (ECAR) were measured in XF media (non-buffered RPMI 1640 containing 25 mM glucose, 2 mM L-glutamine, and 1 mM sodium pyruvate) under basal conditions and in response to 200 μM etomoxir (Tocris), 1 μM oligomycin, 1.5 μM fluoro-carbonyl cyanide phenylhydrazone (FCCP) and 100 nM rotenone+1 μM antimycin A, or 50 ng/mL phorbol 12-myristate 13-acetate (PMA)+500 ng/mL ionomycin (all Sigma) using a 96 well XF or XFe Extracellular Flux Analyzer (EFA) (Seahorse Bioscience).

Adoptive Transfers:

For in vivo memory T cell experiments, ≦1×10⁴ OT-I⁺ CD8⁺ cells/mouse from donor splenocytes were transferred intravenously (i.v.) into congenic recipient mice. Blood samples or spleens were collected at indicated time points and analyzed by flow cytometry. For in vivo survival experiments, 1-2×10⁶ day 6 IL-2 T_{E} treated cells/mouse were injected i.v. into naïve C57BL/6 mice. Cells were recovered two days later from the spleen or lymph nodes and analyzed by flow cytometry or isolated from spleens >3 weeks 6 days after LmOVA infection. For adoptive cellular immunotherapy experiments, 1-5×10⁶ day 6 IL-2 T_{E} treated cells/mouse were injected i.v. into previously EL4-OVA tumor inoculated mice and measured for tumor volume growth.

RT-PCR and Western Blotting:

RNA isolations were done by using the RNeasy kit (Qiagen) and single-strand cDNA was synthesized using the High Capacity cDNA Reverse Transcription Kit (Applied Biosystems). Genomic DNA was extracted using the QIAamp DNA micro kit (Qiagen) to determine mtDNA/nDNA ratios. All RT-PCR was performed with Taqman primers using an Applied Biosystems 7000 sequence detection system. The expression levels of mRNA were normalized to the expression of a housekeeping gene (β-actin). For western blot analyses, cells were washed with ice cold PBS and lysed in 1× lysis buffer (Cell Signaling Technologies) supplemented with 1 mM PMSF. Samples were freeze-thawed 3 times and centrifuged at 20,000×g for 10 min at 4° C. Cleared protein lysate was denatured with LDS loading buffer for 10 min at 70° C. For native lysis, cells were resuspended in native lysis buffer (Life Technologies), supplemented with increasing percentages of digitonin, MgCl, and micrococcal nuclease. After nuclease incubation at RT for 1 h, lysates were cleared by centrifugation at 20,000×g for 30 min at 4° C. For mitochondrial membrane solubilization analyses, both the cleared supernatant and pellet were denatured with LDS loading buffer for 10 min at 70° C. Samples were run on precast 4-12% bis-tris protein gels (Life Technologies). Proteins were transferred onto nitrocellulose membranes using the iBLOT 2 system (Life Technologies). Membranes were blocked with 5% w/v milk and 0.1% Tween-20 in TBS and incubated with the appropriate antibodies in 5% w/v BSA in TBS with 0.1% Tween-20 overnight at 4° C. The following antibodies were used: Opa1 (BD), rodent OXPHOS complex proteins cocktail (Abcam), Calnexin (Santa Cruz), and 13-Actin, Mfn2, Drp1, Drp1^{pS616} (Cell Signaling Technologies). All primary antibody incubations were followed by incubation with secondary HRP-conjugated antibody (Pierce) in 5% milk and 0.1% Tween-20 in TBS and visualized using SuperSignal West Pico or femto Chemiluminescent Substrate (Pierce) on Biomax MR film (Kodak).

Retroviral Transduction:

Activated OT-I splenocytes were transduced with control (empty vector) or Mfn1, Mfn2, Opa1 expressing retrovirus by centrifugation for 90 minutes in media containing hexadimethrine bromide (8 μg/mL; Sigma) and IL-2 (100 U/mL). GFP or human CD8 were markers for retroviral expression.

Cytotoxicity Assay:

EL4-OVA tumor cells were pre-treated with 100 U/mL murine IFN-γ for 24 hours before use. To generate target cells, 1×10⁶ tumor cells were labeled with 0.5 μM Cell Proliferation Dye e670 (eBioscience) in PBS for 8 minutes at room temperature, washed twice with PBS and 10,000 cells were seeded per well in 96-well round bottom plates. IL-2 T_{E} cells treated with DMSO or M1+Mdivi-1 were co-cultured with target cells at the indicated effector/target cell ratios and incubated for 12 hours at 37° C. in 5% CO₂. To generate reference cells, 1×10⁶ tumor cells were labeled with 5 μM Cell Proliferation Dye e670 in PBS and incubated on ice. 10,000 reference cells were added before cells were stained with Po-Pro™-1 dead cell staining dye (Life Technologies). IL-2 T_{E} cell killing efficiency was analyzed by flow cytometry and data defined as percentage of live cells normalized to reference cells.

Statistical Analysis:

Comparisons for two groups were calculated using unpaired two-tailed student's t-tests, comparisons for more than two groups were calculated using one-way ANOVA followed by Bonferroni's multiple comparison tests. Comparisons over time were calculated using two-way ANOVA followed by Bonferroni's multiple comparison tests.

### REFERENCES FOR THE EXAMPLES

Archer, S. L. (2014). Mitochondrial fission and fusion in human diseases. N Engl J Med, 370, 1074.
Baixauli, F., Martin-Cofreces, N. B., Morlino, G., Carrasco, Y. R., Calabia-Linares, C., Veiga, E., Serrador, J. M. & Sanchez-Madrid, F. (2011). The mitochondrial fission factor dynamin-related protein 1 modulates T-cell receptor signalling at the immune synapse. EMBO J, 30, 1238-50.
Benard, G., Bellance, N., James, D., Parrone, P., Fernandez, H., Letellier, T. & Rossignol, R. (2007). Mitochondrial bioenergetics and structural network organization. J Cell Sci, 120, 838-48.
Benard, G. & Rossignol, R. (2008). Ultrastructure of the mitochondrion and its bearing on function and bioenergetics. Antioxid Redox Signal, 10, 1313-42.
Buck, M. D., O'Sullivan, D. & Pearce, E. L. (2015). T cell metabolism drives immunity. J Exp Med, 212, 1345-60.
Campello, S., Lacalle, R. A., Bettella, M., Manes, S., Scorrano, L. & Viola, A. (2006). Orchestration of lymphocyte chemotaxis by mitochondrial dynamics. J Exp Med, 203, 2879-86.
Carrio, R., Bathe, O. F. & Malek, T. R. (2004). Initial antigen encounter programs CD8+ T cells competent to develop into memory cells that are activated in an antigen-free, IL-7- and IL-15-rich environment. J Immunol, 172, 7315-23.
Cassidy-Stone, A., Chipuk, J. E., Ingerman, E., Song, C., Yoo, C., Kuwana, T., Kurth, M. J., Shaw, J. T., Hinshaw, J. E., Green, D. R. & Nunnari, J. (2008). Chemical inhibition of the mitochondrial division dynamin reveals its role in Bax/Bak-dependent mitochondrial outer membrane permeabilization. Dev Cell, 14, 193-204.
Cereghetti, G. M., Stangherlin, A., Martins de Brito, O., Chang, C. R., Blackstone, C., Bernardi, P. & Scorrano, L. (2008). Dephosphorylation by calcineurin regulates translocation of Drp1 to mitochondria. Proc Natl Acad Sci USA, 105, 15803-8.
Chan, D. C. (2012). Fusion and fission: interlinked processes critical for mitochondrial health. Annu Rev Genet, 46, 265-87.
Chang, C. H., Curtis, J. D., Maggi, L. B., Jr., Faubert, B., Villarino, A. V., O'Sullivan, D., Huang, S. C., van der Windt, G. J., Blagih, J., Qiu, J., Weber, J. D., Pearce, E. J., Jones, R. G. & Pearce, E. L. (2013). Posttranscriptional control of T cell effector function by aerobic glycolysis. Cell, 153, 1239-51.
Chang, C. H., Qiu, J., O'Sullivan, D., Buck, M. D., Noguchi, T., Curtis, J. D., Chen, Q., Gindin, M., Gubin, M. M., van der Windt, G. J., Tonc, E., Schreiber, R. D., Pearce, E. J. & Pearce, E. L. (2015). Metabolic Competition in the Tumor Microenvironment Is a Driver of Cancer Progression. Cell, 162, 1229-41.
Chen, H., Detmer, S. A., Ewald, A. J., Griffin, E. E., Fraser, S. E. & Chan, D. C. (2003). Mitofusins Mfn1 and Mfn2 coordinately regulate mitochondrial fusion and are essential for embryonic development. J Cell Biol, 160, 189-200.
Chen, H., McCaffery, J. M. & Chan, D. C. (2007). Mitochondrial fusion protects against neurodegeneration in the cerebellum. Cell, 130, 548-62.
Choi, S. W., Gerencser, A. A. & Nicholls, D. G. (2009). Bioenergetic analysis of isolated cerebrocortical nerve terminals on a microgram scale: spare respiratory capacity and stochastic mitochondrial failure. J Neurochem, 109, 1179-91.
Chouchani, E. T., Pell, V. R., Gaude, E., Aksentijevic, D., Sundier, S. Y., Robb, E. L., Logan, A., Nadtochiy, S. M., Ord, E. N., Smith, A. C., Eyassu, F., Shirley, R., Hu, C. H., Dare, A. J., James, A. M., Rogatti, S., Hartley, R. C., Eaton, S., Costa, A. S., Brookes, P. S., Davidson, S. M., Duchen, M. R., Saeb-Parsy, K., Shattock, M. J., Robinson, A. J., Work, L. M., Frezza, C., Krieg, T. & Murphy, M. P. (2014). Ischaemic accumulation of succinate controls reperfusion injury through mitochondrial ROS. Nature, 515, 431-5.
Cipolat, S., Martins de Brito, O., Dal Zilio, B. & Scorrano, L. (2004). OPA1 requires mitofusin 1 to promote mitochondrial fusion. Proc Natl Acad Sci USA, 101, 15927-32.
Civiletto, G., Varanita, T., Cerutti, R., Gorletta, T., Barbaro, S., Marchet, S., Lamperti, C., Viscomi, C., Scorrano, L. & Zeviani, M. (2015). Opa1 overexpression ameliorates the phenotype of two mitochondrial disease mouse models. Cell Metab, 21, 845-54.
Cogliati, S., Frezza, C., Soriano, M. E., Varanita, T., Quintana-Cabrera, R., Corrado, M., Cipolat, S., Costa, V., Casarin, A., Gomes, L. C., Perales-Clemente, E., Salviati, L., Fernandez-Silva, P., Enriquez, J. A. & Scorrano, L. (2013). Mitochondrial cristae shape determines respiratory chain supercomplexes assembly and respiratory efficiency. Cell, 155, 16071.
Cui, G., Staron, M. M., Gray, S. M., Ho, P. C., Amezquita, R. A., Wu, J. & Kaech, S. M. (2015). IL-7-Induced Glycerol Transport and TAG Synthesis Promotes Memory CD8+ T Cell Longevity. Cell, 161, 750-61.
Dawson, A. G. (1979). Oxidation of cytosolic NADH formed during aerobic metabolism in mammalian cells. Trends in Biochemical Sciences, 4, 171-176.
de Brito, O. M. & Scorrano, L. (2008). Mitofusin 2 tethers endoplasmic reticulum to mitochondria. Nature, 456, 605-10.
Deberardinis, R. J., Lum, J. J. & Thompson, C. B. (2006). Phosphatidylinositol 3-kinase-dependent modulation of carnitine palmitoyltransferase 1A expression regulates lipid metabolism during hematopoietic cell growth. J Biol Chem, 281, 37372-80.
Everts, B., Amiel, E., Huang, S. C., Smith, A. M., Chang, C. H., Lam, W. Y., Redmann, V., Freitas, T. C., Blagih, J., van der Windt, G. J., Artyomov, M. N., Jones, R. G., Pearce, E. L. & Pearce, E. J. (2014). TLR-driven early glycolytic reprogramming via the kinases TBK1-IKKvarepsilon supports the anabolic demands of dendritic cell activation. Nat Immunol, 15, 323-32.
Ferrick, D. A., Neilson, A. & Beeson, C. (2008). Advances in measuring cellular bioenergetics using extracellular flux. Drug Discov Today, 13, 268-74.
Fox, C. J., Hammerman, P. S. & Thompson, C. B. (2005). Fuel feeds function: energy metabolism and the T-cell response. Nat Rev Immunol, 5, 844-52.
Frank, M., Duvezin-Caubet, S., Koob, S., Occhipinti, A., Jagasia, R., Petcherski, A., Ruonala, M. O., Priault, M., Salin, B. & Reichert, A. S. (2012). Mitophagy is triggered by mild oxidative stress in a mitochondrial fission dependent manner. Biochim Biophys Acta, 1823, 2297-310.
Frezza, C., Cipolat, S., Martins de Brito, O., Micaroni, M., Beznoussenko, G. V., Rudka, T., Bartoli, D., Polishuck, R. S., Danial, N. N., De Strooper, B. & Scorrano, L. (2006). OPA1 controls apoptotic cristae remodeling independently from mitochondrial fusion. Cell, 126, 177-89.
Friedman, J. R. & Nunnari, J. (2014). Mitochondrial form and function. Nature, 505, 335-43.
Gomes, L. C., Di Benedetto, G. & Scorrano, L. (2011). During autophagy mitochondria elongate are spared from degradation and sustain cell viability. Nat Cell Biol, 13, 589-98.
Houtkooper, R. H., Pirinen, E. & Auwerx, J. (2012). Sirtuins as regulators of metabolism and healthspan. Nat Rev Mol Cell Biol, 13, 225-38.
Huang, S. C., Everts, B., Ivanova, Y., O'Sullivan, D., Nascimento, M., Smith, A. M., Beatty, W., Love-Gregory, L., Lam, W. Y., O'Neill, C. M., Yan, C., Du, H., Abumrad, N. A., Urban, J. F., Jr., Artyomov, M. N., Pearce, E. L. & Pearce, E. J. (2014). Cell-intrinsic lysosomal lipolysis is essential for alternative activation of macrophages. Nat Immunol, 15, 846-55.
Ingerman, E., Perkins, E. M., Marino, M., Mears, J. A., McCaffery, J. M., Hinshaw, J. E. & Nunnari, J. (2005). Dnm1 forms spirals that are structurally tailored to fit mitochondria. J Cell Biol, 170, 1021-7.
Ishihara, N., Otera, H., Oka, T. & Mihara, K. (2013). Regulation and physiologic functions of GTPases in mitochondrial fusion and fission in mammals. Antioxid Redox Signal, 19, 389-99.
Kaech, S. M., Tan, J. T., Wherry, E. J., Konieczny, B. T., Surh, C. D. & Ahmed, R. (2003). Selective expression of the interleukin 7 receptor identifies effector CD8 T cells that give rise to long-lived memory cells. Nat Immunol, 4, 1191-8.
Krawczyk, C. M., Holowka, T., Sun, J., Blagih, J., Amiel, E., DeBerardinis, R. J., Cross, J. R., Jung, E., Thompson, C. B., Jones, R. G. & Pearce, E. J. (2010). Toll-like receptor-induced changes in glycolytic metabolism regulate dendritic cell activation. Blood, 115, 4742-9.
Labrousse, A. M., Zappaterra, M. D., Rube, D. A. & van der Bliek, A. M. (1999). C. elegans dynamin-related protein DRP-1 controls severing of the mitochondrial outer membrane. Mol Cell, 4, 815-26.
Liesa, M. & Shirihai, O. S. (2013). Mitochondrial dynamics in the regulation of nutrient utilization and energy expenditure. Cell Metab, 17, 491-506.
Maclver, N. J., Michalek, R. D. & Rathmell, J. C. (2013). Metabolic regulation of T lymphocytes. Annu Rev Immunol, 31, 259-83.
Marsboom, G., Toth, P. T., Ryan, J. J., Hong, Z., Wu, X., Fang, Y. H., Thenappan, T., Piao, L., Zhang, H. J., Pogoriler, J., Chen, Y., Morrow, E., Weir, E. K., Rehman, J. & Archer, S. L. (2012). Dynamin-related protein 1-mediated mitochondrial mitotic fission permits hyperproliferation of vascular smooth muscle cells and offers a novel therapeutic target in pulmonary hypertension. Circ Res, 110, 1484-97.
Maus, M. V., Fraietta, J. A., Levine, B. L., Kalos, M., Zhao, Y. & June, C. H. (2014). Adoptive immunotherapy for cancer or viruses. Annu Rev Immunol, 32, 189-225.
Mishra, P., Carelli, V., Manfredi, G. & Chan, D. C. (2014). Proteolytic cleavage of Opa1 stimulates mitochondrial inner membrane fusion and couples fusion to oxidative phosphorylation. Cell Metab, 19, 630-41.
Mishra, P. & Chan, D. C. (2016). Metabolic regulation of mitochondrial dynamics. J Cell Biol, 212, 379-87.
Nicholls, D. G. (2009). Spare respiratory capacity, oxidative stress and excitotoxicity. Biochem Soc Trans, 37, 1385-8.
Nicholls, D. G., Darley-Usmar, V. M., Wu, M., Jensen, P. B., Rogers, G. W. & Ferrick, D. A. (2010). Bioenergetic profile experiment using C2C12 myoblast cells. J Vis Exp.
Nunnari, J. & Suomalainen, A. (2012). Mitochondria: in sickness and in health. Cell, 148, 114559.
O'Sullivan, D. & Pearce, E. L. (2015). Targeting T cell metabolism for therapy. Trends Immunol, 36, 71-80.
O'Sullivan, D., van der Windt, G. J., Huang, S. C., Curtis, J. D., Chang, C. H., Buck, M. D., Qiu, J., Smith, A. M., Lam, W. Y., DiPlato, L. M., Hsu, F. F., Birnbaum, M. J., Pearce, E. J. & Pearce, E. L. (2014). Memory CD8(+) T cells use cell-intrinsic lipolysis to support the metabolic programming necessary for development. Immunity, 41, 75-88.
Patten, D. A., Wong, J., Khacho, M., Soubannier, V., Mailloux, R. J., Pilon-Larose, K., MacLaurin, J. G., Park, D. S., McBride, H. M., Trinkle-Mulcahy, L., Harper, M. E., Germain, M. & Slack, R. S. (2014). OPA1-dependent cristae modulation is essential for cellular adaptation to metabolic demand. EMBO J, 33, 2676-91.
Pearce, E. L. & Pearce, E. J. (2013). Metabolic pathways in immune cell activation and quiescence. Immunity, 38, 633-43.
Pearce, E. L., Poffenberger, M. C., Chang, C. H. & Jones, R. G. (2013). Fueling immunity: insights into metabolism and lymphocyte function. Science, 342, 1242454.
Pearce, E. L., Walsh, M. C., Cejas, P. J., Harms, G. M., Shen, H., Wang, L. S., Jones, R. G. & Choi, Y. (2009). Enhancing CD8 T-cell memory by modulating fatty acid metabolism. Nature, 460, 103-7.
Rambold, A. S., Cohen, S. & Lippincott-Schwartz, J. (2015). Fatty acid trafficking in starved cells: regulation by lipid droplet lipolysis, autophagy, and mitochondrial fusion dynamics. Dev Cell, 32, 678-92.
Rambold, A. S., Kostelecky, B., Elia, N. & Lippincott-Schwartz, J. (2011a). Tubular network formation protects mitochondria from autophagosomal degradation during nutrient starvation. Proc Natl Acad Sci USA, 108, 10190-5.
Rambold, A. S., Kostelecky, B. & Lippincott-Schwartz, J. (2011 b). Fuse or die: Shaping mitochondrial fate during starvation. Commun Integr Biol, 4, 752-4.
Restifo, N. P., Dudley, M. E. & Rosenberg, S. A. (2012). Adoptive immunotherapy for cancer: harnessing the T cell response. Nat Rev Immunol, 12, 269-81.
Roth, D., Krammer, P. H. & Gulow, K. (2014). Dynamin related protein 1-dependent mitochondrial fission regulates oxidative signalling in T cells. FEBS Lett, 588, 1749-54.
Samant, S. A., Zhang, H. J., Hong, Z., Pillai, V. B., Sundaresan, N. R., Wolfgeher, D., Archer, S. L., Chan, D. C. & Gupta, M. P. (2014). SIRT3 deacetylates and activates OPA1 to regulate mitochondrial dynamics during stress. Mol Cell Biol, 34, 807-19.
Santel, A., Frank, S., Gaume, B., Herrler, M., Youle, R. J. & Fuller, M. T. (2003). Mitofusin-1 protein is a generally expressed mediator of mitochondrial fusion in mammalian cells. J Cell Sci, 116, 2763-74.
Schluns, K. S., Williams, K., Ma, A., Zheng, X. X. & Lefrancois, L. (2002). Cutting edge: requirement for IL-15 in the generation of primary and memory antigen-specific CD8 T cells. J Immunol, 168, 4827-31.
Sebastian, D., Hernandez-Alvarez, M. I., Segales, J., Sorianello, E., Munoz, J. P., Sala, D., Waget, A., Liesa, M., Paz, J. C., Gopalacharyulu, P., Oresic, M., Pich, S., Burcelin, R., Palacin, M. & Zorzano, A. (2012). Mitofusin 2 (Mfn2) links mitochondrial and endoplasmic reticulum function with insulin signaling and is essential for normal glucose homeostasis. Proc Natl Acad Sci USA, 109, 5523-8.
Sena, L. A., Li, S., Jairaman, A., Prakriya, M., Ezponda, T., Hildeman, D. A., Wang, C. R., Schumacker, P. T., Licht, J. D., Perlman, H., Bryce, P. J. & Chandel, N. S. (2013). Mitochondria are required for antigen-specific T cell activation through reactive oxygen species signaling. Immunity, 38, 225-36.
Serasinghe, M. N., Wieder, S. Y., Renault, T. T., Elkholi, R., Asciolla, J. J., Yao, J. L., Jabado, O., Hoehn, K., Kageyama, Y., Sesaki, H. & Chipuk, J. E. (2015). Mitochondrial division is requisite to RAS-induced transformation and targeted by oncogenic MAPK pathway inhibitors. Mol Cell, 57, 521-36.
Smith-Garvin, J. E., Koretzky, G. A. & Jordan, M. S. (2009). T cell activation. Annu Rev Immunol, 27, 591-619.
Taguchi, N., Ishihara, N., Jofuku, A., Oka, T. & Mihara, K. (2007). Mitotic phosphorylation of dynamin-related GTPase Drp1 participates in mitochondrial fission. J Biol Chem, 282, 11521-9.
van der Windt, G. J., Everts, B., Chang, C. H., Curtis, J. D., Freitas, T. C., Amiel, E., Pearce, E. J. & Pearce, E. L. (2012). Mitochondrial respiratory capacity is a critical regulator of CD8+ T cell memory development. Immunity, 36, 68-78.
van der Windt, G. J., O'Sullivan, D., Everts, B., Huang, S. C., Buck, M. D., Curtis, J. D., Chang, C. H., Smith, A. M., Ai, T., Faubert, B., Jones, R. G., Pearce, E. J. & Pearce, E. L. (2013). CD8 memory T cells have a bioenergetic advantage that underlies their rapid recall ability. Proc Natl Acad Sci USA, 110, 14336-41.
Vander Heiden, M. G., Cantley, L. C. & Thompson, C. B. (2009). Understanding the Warburg effect: the metabolic requirements of cell proliferation. Science, 324, 1029-33.
Varanita, T., Soriano, M. E., Romanello, V., Zaglia, T., Quintana-Cabrera, R., Semenzato, M., Menabo, R., Costa, V., Civiletto, G., Pesce, P., Viscomi, C., Zeviani, M., Di Lisa, F., Mongillo, M., Sandri, M. & Scorrano, L. (2015). The OPA1-dependent mitochondrial cristae remodeling pathway controls atrophic, apoptotic, and ischemic tissue damage. Cell Metab, 21, 834-44.
Wai, T. & Langer, T. (2016). Mitochondrial Dynamics and Metabolic Regulation. Trends Endocrinol Metab, 27, 105-17.
Wakabayashi, J., Zhang, Z., Wakabayashi, N., Tamura, Y., Fukaya, M., Kensler, T. W., Iijima, M. & Sesaki, H. (2009). The dynamin-related GTPase Drp1 is required for embryonic and brain development in mice. J Cell Biol, 186, 805-16.
Wang, D., Wang, J., Bonamy, G. M., Meeusen, S., Brusch, R. G., Turk, C., Yang, P. & Schultz, P. G. (2012). A small molecule promotes mitochondrial fusion in mammalian cells. Angew Chem Int Ed Engl, 51, 9302-5.
Wang, L., Ishihara, T., lbayashi, Y., Tatsushima, K., Setoyama, D., Hanada, Y., Takeichi, Y., Sakamoto, S., Yokota, S., Mihara, K., Kang, D., Ishihara, N., Takayanagi, R. & Nomura, M. (2015). Disruption of mitochondrial fission in the liver protects mice from diet-induced obesity and metabolic deterioration. Diabetologia, 58, 2371-80.
Wang, R. & Green, D. R. (2012). Metabolic checkpoints in activated T cells. Nat Immunol, 13, 907-15.
Williams, M. A. & Bevan, M. J. (2007). Effector and memory CTL differentiation. Annu Rev Immunol, 25, 171-92.
Yadava, N. & Nicholls, D. G. (2007). Spare respiratory capacity rather than oxidative stress regulates glutamate excitotoxicity after partial respiratory inhibition of mitochondrial complex I with rotenone. J Neurosci, 27, 7310-7.
Youle, R. J. & Karbowski, M. (2005). Mitochondrial fission in apoptosis. Nat Rev Mol Cell Biol, 6, 657-63.
Youle, R. J. & van der Bliek, A. M. (2012). Mitochondrial fission, fusion, and stress. Science, 337, 1062-5.
Yu, T., Robotham, J. L. & Yoon, Y. (2006). Increased production of reactive oxygen species in hyperglycemic conditions requires dynamic change of mitochondrial morphology. Proc Natl Acad Sci USA, 103, 2653-8.
Zanna C, Ghelli A, Porcelli A M, Karbowski M, Youle R J, Schimpf S, Wissinger B, Pinti M, Cossarizza A, Vidoni S, Valentino M L, Rugolo M, Carelli V. OPA1 mutations associated with dominant atrophy impair oxidative phosphorylation and mitochondrial fusion. (2008). Brain, 131, 352-67.
Zhang, Z., Wakabayashi, N., Wakabayashi, J., Tamura, Y., Song, W. J., Sereda, S., Clerc, P., Polster, B. M., Aja, S. M., Pletnikov, M. V., Kensler, T. W., Shirihai, O. S., Iijima, M., Hussain, M. A. & Sesaki, H. (2011). The dynamin-related GTPase Opa1 is required for glucose-stimulated ATP production in pancreatic beta cells. Mol Biol Cell, 22, 2235-45.
Zorzano, A., Liesa, M., Sebastian, D., Segales, J. & Palacin, M. (2010). Mitochondrial fusion proteins: dual regulators of morphology and metabolism. Semin Cell Dev Biol, 21, 56674.

What is claimed is:

1. A method to promote T-cell longevity, the method comprising culturing T cells in the presence of a composition comprising one or more compounds to promote mitochondrial fusion and/or mitochondrial structural remodeling including cristae.

2. The method of claim 1, wherein the compound to promote mitochondrial fusion is M1.

3. The method of claim 1, wherein the composition further comprises one or more compounds to inhibit fission.

4. The method of claim 3, wherein the compound to inhibit fission is Mdivi-1.

5. The method of claim 1, wherein the T cells are cultured in the absence of one or more compounds to promote mitochondrial fusion prior to culturing in the presence of a composition comprising one or more compounds to promote mitochondrial fusion and/or mitochondrial structural remodeling including cristae.

6. The method of claim 5, wherein prior to the addition of the one or more compounds to promote mitochondrial fusion and/or mitochondrial structural remodeling including cristae, the T cells are cultured in the presence of IL2.

7. The method of claim 5, wherein prior to the addition of the one or more compounds to promote mitochondrial fusion and/or mitochondrial structural remodeling including cristae, the T cells are cultured in the presence of an antigen.

8. The method of claim 1, wherein the T cells are isolated from a subject prior to culturing in the presence of a composition comprising one or more compounds to promote mitochondrial fusion and/or mitochondrial structural remodeling including cristae.

9. The method of claim 1, wherein the T cells exhibit increased cytokine production.

10. The method of claim 1, wherein the T cells exhibit improved function.

11. A method to improve adoptive cellular immunotherapy in a subject, the method comprising administering to the subject a therapeutic composition comprising T cells that have been cultured in the presence of a composition comprising one or more compounds to promote mitochondrial fusion and/or mitochondrial structural remodeling including cristae.

12. The method of claim 11, wherein the T cells are present in the subject more than 2 days following administration.

13. The method of claim 11, wherein the T cells are isolated from the subject prior to culturing in the presence of a composition comprising one or more compounds to promote mitochondrial fusion and/or mitochondrial structural remodeling including cristae.

14. The method of claim 11, wherein the compound to promote mitochondrial fusion is M1.

15. The method of claim 11, wherein the composition comprising one or more compounds to promote mitochondrial fusion further comprises Mdivi-1.

16. A method to reduce tumor growth in a subject, the method comprising administering to the subject a therapeutic composition comprising T cells that have been cultured in the presence of a composition comprising one or more compounds to promote mitochondrial fusion and/or mitochondrial structural remodeling including cristae.

17. The method of claim 16, wherein the T cells are present in the subject more than 2 days following administration.

18. The method of claim 16, wherein the T cells are isolated from the subject prior to culturing in the presence of a composition comprising one or more compounds to promote mitochondrial fusion and/or mitochondrial structural remodeling including cristae.

19. The method of claim 16, wherein tumor growth is reduced by at least 50%.

20. The method of claim 16, wherein the compound to promote mitochondrial fusion is M1.
